SEQUENCE LISTING (32 sequences)

SEQ ID NO: 1 - DNA, 20 bp, Mus musculus
  source 1..20: mol_type = genomic DNA
ccccatcgtctatgccttcc

SEQ ID NO: 2 - DNA, 20 bp, Mus musculus
  source 1..20: mol_type = genomic DNA
catcggaagtggtcgttcca

SEQ ID NO: 3 - DNA, 22 bp, Mus musculus
  source 1..22: mol_type = genomic DNA
accagagcgaaagcatttgcca

SEQ ID NO: 4 - DNA, 22 bp, Mus musculus
  source 1..22: mol_type = genomic DNA
atcgccagtcggcatcgtttat

SEQ ID NO: 5 - DNA, 19 bp, Mus musculus
  source 1..19: mol_type = genomic DNA
gggagcgcatcgagttttg

SEQ ID NO: 6 - DNA, 20 bp, Mus musculus
  source 1..20: mol_type = genomic DNA
tacgctatgcacctcactgc

SEQ ID NO: 7 - DNA, 21 bp, Mus musculus
  source 1..21: mol_type = genomic DNA
cagcgagttcaaagacccaga

SEQ ID NO: 8 - DNA, 20 bp, Mus musculus
  source 1..20: mol_type = genomic DNA
gcagagcaatggtgtcgttc

SEQ ID NO: 9 - DNA, 21 bp, Mus musculus
  source 1..21: mol_type = genomic DNA
gccagagcaagaggcaggtat

SEQ ID NO: 10 - DNA, 20 bp, Mus musculus
  source 1..20: mol_type = genomic DNA
tcccaaaggctttctcacgg

SEQ ID NO: 11 - DNA, 21 bp, Mus musculus
  source 1..21: mol_type = genomic DNA
atctttagcctcttggcggtg

SEQ ID NO: 12 - DNA, 21 bp, Mus musculus
  source 1..21: mol_type = genomic DNA
gacccagaggacagcaatgat

SEQ ID NO: 13 - DNA, 20 bp, Mus musculus
  source 1..20: mol_type = genomic DNA
gctgtagaccgatacctgcg

SEQ ID NO: 14 - DNA, 20 bp, Mus musculus
  source 1..20: mol_type = genomic DNA
ggaaactagccagcaaaggc

SEQ ID NO: 15 - DNA, 20 bp, Mus musculus
  source 1..20: mol_type = genomic DNA
gcacagtcaaggccgagaat

SEQ ID NO: 16 - DNA, 20 bp, Mus musculus
  source 1..20: mol_type = genomic DNA
gccttctccatggtggtgaa

SEQ ID NO: 17 - DNA, 20 bp, Mus musculus
  source 1..20: mol_type = genomic DNA
tggccgggaatgatgagaac

SEQ ID NO: 18 - DNA, 20 bp, Mus musculus
  source 1..20: mol_type = genomic DNA
tgaaactcttgcctcgtccg

SEQ ID NO: 19 - DNA, 19 bp, Mus musculus
  source 1..19: mol_type = genomic DNA
tgcctgactccttgggacc

SEQ ID NO: 20 - DNA, 22 bp, Mus musculus
  source 1..22: mol_type = genomic DNA
tagtgagcttcttcctcaagca

SEQ ID NO: 21 - DNA, 21 bp, Mus musculus
  source 1..21: mol_type = genomic DNA
tccacactttccacactctcg

SEQ ID NO: 22 - DNA, 21 bp, Mus musculus
  source 1..21: mol_type = genomic DNA
ctttctgcatctccagccttc

SEQ ID NO: 23 - DNA, 20 bp, Mus musculus
  source 1..20: mol_type = genomic DNA
gctaccttggagcctcagtc

SEQ ID NO: 24 - DNA, 21 bp, Mus musculus
  source 1..21: mol_type = genomic DNA
agggttaagctcacactgctc

SEQ ID NO: 25 - protein, 326 aa, Homo sapiens
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

SEQ ID NO: 26 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 27 - protein, 332 aa, Homo sapiens
MLLETQDALYVALELVIAALSVAGNVLVCAAVGTANTLQTPTNYFLVSLAAADVAVGLFAIPFAITISLGFCTDFYGCLFLACFVLVLTQSSIFSLLAVAVDRYLAICVPLRYKSLVTGTRARGVIAVLWVLAFGIGLTPFLGWNSKDSATNNCTEPWDGTTNESCCLVKCLFENVVPMSYMVYFNFFGCVLPPLLIMLVIYIKIFLVACRQLQRTELMDHSRTTLQREIHAAKSLAMIVGIFALCWLPVHAVNCVTLFQPAQGKNKPKWAMNMAILLSHANSVVNPIVYAYRNRDFRYTFHKIISRYLLCQADVKSGNGQAGVQPALGVGL

SEQ ID NO: 28 - protein, 318 aa, Homo sapiens
MPNNSTALSLANVTYITMEIFIGLCAIVGNVLVICVVKLNPSLQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVGLTPMFGWNMKLTSEYHRNVTFLSCQFVSVMRMDYMVYFSFLTWIFIPLVVMCAIYLDIFYIIRNKLSLNLSNSKETGAFYGREFKTAKSLFLVLFLFALSWLPLSIINCIIYFNGEVPQLVLYMGILLSHANSMMNPIVYAYKIKKFKETYLLILKACVVCHPSDSLDTSIEKNSE

SEQ ID NO: 29 - protein, 326 aa, Mus musculus
MPPYISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKTVVTQRRAAVAIAGCWILSLVVGLTPMFGWNNLSEVEQAWIANGSVGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPTCQKPSILIYIAIFLTHGNSAMNPIVYAFRIHKFRVTFLKIWNDHFRCQPKPPIEEDIPEEKADD

SEQ ID NO: 30 - protein, 410 aa, Mus musculus
MGSSVYIMVELAIAVLAILGNVLVCWAVWINSNLQNVTNFFVVSLAAADIAVGVLAIPFAITISTGFCAACHGCLFFACFVLVLTQSSIFSLLAIAIDRYIAIRIPLRYNGLVTGMRAKGIIAICWVLSFAIGLTPMLGWNNCSQKDENSTKTCGEGRVTCLFEDVVPMNYMVYYNFFAFVLLPLLLMLAIYLRIFLAARRQLKQMESQPLPGERTRSTLQKEVHAAKSLAIIVGLFALCWLPLHIINCFTFFCSTCQHAPPWLMYLAIILSHSNSVVNPFIYAYRIREFRQTFRKIIRTHVLRRQEPFRAGGSSAWALAAHSTEGEQVSLRLNGHPLGVWANGSAPHSGRRPNGYTLGPGGGGSTQGSPGDVELLTQEHQEGQEHPGLGDHLAQGRVGTASWSSEFAPS

SEQ ID NO: 31 - protein, 332 aa, Mus musculus
MQLETQDALYVALELVIAALAVAGNVLVCAAVGASSALQTPTNYFLVSLATADVAVGLFAIPFAITISLGFCTDFHGCLFLACFVLVLTQSSIFSLLAVAVDRYLAIRVPLRYKGLVTGTRARGIIAVLWVLAFGIGLTPFLGWNSKDSATSNCTELGDGIANKSCCPVTCLFENVVPMSYMVYFNFFGCVLPPLLIMLVIYIKIFMVACKQLQRMELMDHSRTTLQREIHAAKSLAMIVGIFALCWLPVHAINCITLFHPALAKDKPKWVMNVAILLSHANSVVNPIVYAYRNRDFRYSFHKIISRYVLCQAETKGGSGQAGAQSTLSLGL

SEQ ID NO: 32 - protein, 319 aa, Mus musculus
MEADNTTETDWLNITYITMEAAIGLCAVVGNMLVIWVVKLNPTLRTTTVYFIVSLALADIAVGVLVIPLAIAVSLQVKMHFYACLFMSCVLLIFTHASIMSLLAIAVHRYLRVKLTVRYRTVTTQRRIWLFLGLCWLVSFLVGLTPMFGWNRKATLASSQNSSTLLCHFRSVVSLDYMVFFSFITWILVPLVVMCIIYLDIFYIIRNKLSQNLTGFRETRAFYGREFKTAKSLFLVLFLFALCWLPLSIINFVSYFDVKIPDVAMCLGILLSHANSMMNPIVYACKIKKFKETYFLILRAVRLCQTSDSLDSNMEQTTE

(19) United States
(12) Patent Application Publication
(10) Pub. No.: US 2025/0152616 A1
(43) Pub. Date: May 15, 2025

(54) COMPOSITIONS FOR AND METHODS OF TREATING, PREVENTING, AND/OR MITIGATING PAIN

(71) Applicant: Duke University, Durham, NC (US)

(72) Inventors: Shyni Varghese, Durham, NC (US); Yu Ru Shih, Durham, NC (US)

(21) Appl. No.: 18/726,920

(22) PCT Filed: Jan. 5, 2023
(86) PCT No.: PCT/US2023/060141

§ 371 Date: Jul. 5, 2024

Related U.S. Application Data
(60) Provisional application No. 63/297,216, filed on Jan. 6, 2022.

(51) Int. Cl. A61K 31/7076; A61K 9/06; A61K 45/06; A61K 47/10; A61P 29/00

(52) U.S. Cl. CPC A61K 31/7076; A61K 9/06; A61K 45/06; A61K 47/10; A61P 29/00

(57) ABSTRACT

Disclosed herein are compositions for and methods of treating, preventing, and/or mitigating pain, including both acute and chronic pain, post-surgical pain, cancer pain, injury pain, and pain associated with bone fracture. Disclosed herein are compositions for and methods of providing non-opioid analgesia in a subject in need thereof.

### CROSS-REFERENCE TO RELATED APPLICATION

This application claims priority to U.S. Provisional Application No. 63/297,216 filed 6 Jan. 2022, which is incorporated herein in its entirety.

### STATEMENT REGARDING FEDERALLY SPONSORED RESEARCH OR DEVELOPMENT

This application was made with government support under Grant Nos. R01AR071552 and R01AR079189 awarded by National Institutes of Health. The government has certain rights in this invention.

### REFERENCE TO THE SEQUENCE LISTING

The Sequence Listing submitted 5 Jan. 2023 as an XML file named “20_872_WO2_Sequence_Listing”, created on 3 Jan. 2023 and having a size of 35 KB kilobytes is hereby incorporated by reference pursuant to 37 C.F.R. § 1.52 (e) (5).

### BACKGROUND OF THE INVENTION

Skeletal injuries are a major cause of morbidities worldwide with bone fractures accounting for a substantial portion. As one of the most prevalent traumas, bone injuries cause intense acute pain and loss of function. Patients suffering from bone fractures and undergoing surgery experience different levels of pain throughout the healing process requiring pain-mitigating interventions. Furthermore, a considerable number of bone fractures suffer from delayed healing, and unresolved acute pain may transition to chronic and maladaptive pain. An estimated 3.9 million bone fractures occur in the United States with an annual economic burden of $116.9 billion in combined medical expenses and work loss. Current management of pain involves treatment with NSAIDs and opioids, however, these analgesics have substantial drawbacks including delaying healing, systemic side effects, and potential for addiction. Hence, a therapeutic approach that concomitantly attenuates pain locally and actively promotes healing would address a significant clinical problem and improve the overall functional outcome for patients.

Accordingly, there exists a need in the art for compositions for and methods of treating, preventing, and/or mitigating pain, including chronic pain, acute pain, post-surgical pain, injury pain, and pain associated with bone fracture.

### BRIEF SUMMARY OF THE INVENTION

Disclosed herein is a biomaterial comprising a therapeutically effective amount of adenosine (ADO) for treating, preventing, and/or mitigating pain. Disclosed herein is a biomaterial comprising macroporous scaffolds. Disclosed herein is a biomaterial comprising macroporous scaffolds, wherein the scaffolds comprise 3-acrylamido phenylboronic acid (3-APBA). Disclosed herein is a biomaterial comprising macroporous scaffolds, wherein the scaffolds comprise a hydrogel comprising 3-acrylamido phenylboronic acid (3-APBA). Disclosed herein is a biomaterial comprising macroporous scaffolds comprising adenosine. Disclosed herein is a biomaterial comprising macroporous scaffolds, wherein the scaffolds comprise 3-acrylamido phenylboronic acid (3-APBA) and adenosine. Disclosed herein is a biomaterial comprising macroporous scaffolds, wherein the scaffolds comprise a hydrogel comprises 3-acrylamido phenylboronic acid (3-APBA) and adenosine. Disclosed herein is a biomaterial comprising macroporous scaffolds, wherein the scaffolds comprise adenosine. Disclosed herein is a biomaterial comprising 3-APBA-conjugated macroporous scaffolds, wherein the scaffolds comprise adenosine.

Disclosed herein is a biomaterial comprising a hydrogel. Disclosed herein is a biomaterial comprising a hydrogel, wherein the hydrogel comprises macroporous scaffolds. Disclosed herein is a biomaterial comprising a hydrogel, wherein the hydrogel comprises macroporous scaffolds comprising 3-acrylamido phenylboronic acid (3-APBA). Disclosed herein is a biomaterial comprising a hydrogel comprising adenosine. Disclosed herein is a biomaterial comprising a hydrogel, wherein the hydrogel comprises 3-acrylamido phenylboronic acid (3-APBA) and adenosine. Disclosed herein is a biomaterial comprising 3-APBA-conjugated hydrogel, wherein the hydrogel comprises macroporous scaffolds and adenosine. In an aspect, 3-APBA can be conjugated to the disclosed scaffolds.

Disclosed herein is a biomaterial comprising poly(ethylene glycol)-co-6 aminocaproic acid macroporous scaffolds. Disclosed herein is a biomaterial comprising poly(ethylene glycol)-co-6 aminocaproic acid macroporous scaffolds, wherein the scaffolds comprise 3-acrylamido phenylboronic acid (3-APBA). Disclosed herein is a biomaterial comprising macroporous scaffolds, wherein the scaffolds comprise a poly(ethylene glycol)-co-6 aminocaproic acid hydrogel comprising 3-acrylamido phenylboronic acid (3-APBA). Disclosed herein is a biomaterial comprising poly(ethylene glycol)-co-6 aminocaproic acid macroporous scaffolds comprising adenosine. Disclosed herein is a biomaterial comprising poly(ethylene glycol)-co-6 aminocaproic acid macroporous scaffolds, wherein the scaffolds comprise 3-acrylamido phenylboronic acid (3-APBA) and adenosine. Disclosed herein is a biomaterial comprising macroporous scaffolds, wherein the scaffolds comprise a poly(ethylene glycol)-co-6 aminocaproic acid hydrogel comprises 3-acrylamido phenylboronic acid (3-APBA) and adenosine. Disclosed herein is a biomaterial comprising poly(ethylene glycol)-co-6 aminocaproic acid macroporous scaffolds, wherein the scaffolds comprise adenosine. Disclosed herein is a biomaterial comprising 3-APBA-conjugated poly(ethylene glycol)-co-6 aminocaproic acid macroporous scaffolds, wherein the scaffolds comprise adenosine. In an aspect, 3-APBA can be conjugated to the disclosed scaffolds.

Disclosed herein is a biomaterial comprising a poly(ethylene glycol)-co-6 aminocaproic acid hydrogel. Disclosed herein is a biomaterial comprising a poly(ethylene glycol)-co-6 aminocaproic acid hydrogel, wherein the hydrogel comprises macroporous scaffolds. Disclosed herein is a biomaterial comprising a poly(ethylene glycol)-co-6 aminocaproic acid hydrogel, wherein the hydrogel comprises macroporous scaffolds comprising 3-acrylamido phenylboronic acid (3-APBA). Disclosed herein is a biomaterial comprising a poly(ethylene glycol)-co-6 aminocaproic acid hydrogel comprising adenosine. Disclosed herein is a biomaterial comprising a poly(ethylene glycol)-co-6 aminocaproic acid hydrogel, wherein the hydrogel comprises 3-acrylamido phenylboronic acid (3-APBA) and adenosine. Disclosed herein is a biomaterial comprising poly(ethylene glycol)-co-6 aminocaproic acid hydrogel-3-APBA-conjugated, wherein the hydrogel comprises macroporous scaffolds and adenosine. In an aspect, 3-APBA can be conjugated to the disclosed scaffolds.

Disclosed herein is a biomaterial comprising a poly(ethylene glycol)-co-6 aminocaproic acid macroporous hydrogel, wherein the macroporous hydrogel are functionalized with 3-acrylamido phenylboronic acid (3-APBA). Disclosed herein is a biomaterial comprising a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine.

Disclosed herein is a pharmaceutical composition comprising a disclosed biomaterial. Disclosed herein is a pharmaceutical composition comprising a disclosed biomaterial and one or more therapeutic agents. Disclosed herein is a pharmaceutical composition comprising a disclosed hydrogel. Disclosed herein is a pharmaceutical composition comprising a disclosed hydrogel and one or more therapeutic agents. Disclosed herein is a pharmaceutical composition comprising a disclosed biomaterial comprising a therapeutically effective amount of adenosine. Disclosed herein is a pharmaceutical composition comprising a disclosed biomaterial comprising a therapeutically effective amount of adenosine and one or more therapeutic agents. Disclosed herein is a pharmaceutical composition comprising a disclosed hydrogel comprising a therapeutically effective amount of adenosine. Disclosed herein is a pharmaceutical composition comprising a disclosed hydrogel comprising a therapeutically effective amount of adenosine and one or more therapeutic agents.

Disclosed herein is a pharmaceutical composition comprising a biomaterial comprising poly(ethylene glycol)-co-6 aminocaproic acid macroporous scaffolds. Disclosed herein is a pharmaceutical composition comprising a biomaterial comprising poly(ethylene glycol)-co-6 aminocaproic acid macroporous scaffolds, wherein the scaffolds comprise 3-acrylamido phenylboronic acid (3-APBA). Disclosed herein is a pharmaceutical composition comprising a biomaterial comprising macroporous scaffolds, wherein the scaffolds comprise a poly(ethylene acid hydrogel comprising 3-acrylamido phenylboronic acid (3-APBA). Disclosed herein is a pharmaceutical composition comprising a biomaterial comprising poly(ethylene glycol)-co-6 aminocaproic acid macroporous scaffolds comprising adenosine. Disclosed herein is a pharmaceutical composition comprising a biomaterial comprising poly(ethylene glycol)-co-6 aminocaproic acid macroporous scaffolds, wherein the scaffolds comprise 3-acrylamido phenylboronic acid (3-APBA) and adenosine. Disclosed herein is a pharmaceutical composition comprising a biomaterial comprising macroporous scaffolds, wherein the scaffolds comprise a poly(ethylene glycol)-co-6 aminocaproic acid hydrogel comprises 3-acrylamido phenylboronic acid (3-APBA) and adenosine. Disclosed herein is a pharmaceutical composition comprising a biomaterial comprising poly(ethylene glycol)-co-6 aminocaproic acid macroporous scaffolds, wherein the scaffolds comprise adenosine. Disclosed herein is a pharmaceutical composition comprising a biomaterial comprising 3-APBA-conjugated poly(ethylene glycol)-co-6 aminocaproic acid macroporous scaffolds, wherein the scaffolds comprise adenosine.

Disclosed herein is a pharmaceutical composition comprising a biomaterial comprising a poly(ethylene glycol)-co-6 aminocaproic acid hydrogel. Disclosed herein is a pharmaceutical composition comprising a biomaterial comprising a poly(ethylene glycol)-co-6 aminocaproic acid hydrogel, wherein the hydrogel comprises macroporous scaffolds. Disclosed herein is a pharmaceutical composition comprising a biomaterial comprising a poly(ethylene glycol)-co-6 aminocaproic acid hydrogel, wherein the hydrogel comprises macroporous scaffolds comprising 3-acrylamido phenylboronic acid (3-APBA). Disclosed herein is a pharmaceutical composition comprising a biomaterial comprising a poly(ethylene glycol)-co-6 aminocaproic acid hydrogel comprising adenosine. Disclosed herein is a pharmaceutical composition comprising a biomaterial comprising a poly(ethylene glycol)-co-6 aminocaproic acid hydrogel, wherein the hydrogel comprises 3-acrylamido phenylboronic acid (3-APBA) and adenosine. Disclosed herein is a pharmaceutical composition comprising a biomaterial comprising poly(ethylene glycol)-co-6 aminocaproic acid hydrogel-3-APBA-conjugated, wherein the hydrogel comprises macroporous scaffolds and adenosine. In an aspect, 3-APBA can be conjugated to the disclosed scaffolds.

Disclosed herein is a pharmaceutical composition comprising a biomaterial comprising a poly(ethylene glycol)-co-6 aminocaproic acid macroporous hydrogel, wherein the macroporous hydrogel are functionalized with 3-acrylamido phenylboronic acid (3-APBA).

Disclosed herein is a pharmaceutical composition comprising a biomaterial comprising a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine.

Disclosed herein is a kit comprising one or more components and/or reagents for use in a disclosed method of treating, preventing, and/or mitigating pain. Disclosed herein is a kit comprising one or more components and/or reagents for use in a disclosed method of improving movement. Disclosed herein is a method of treating, preventing, and/or mitigating pain in a subject, the method comprising administering to a subject a biomaterial comprising a therapeutically effective amount of adenosine.

Disclosed herein is a method of treating, preventing, and/or mitigating pain in a subject, the method comprising administering to a subject a biomaterial comprising a therapeutically effective amount of adenosine, wherein the subject's pain is reduced. Disclosed herein is a method of treating, preventing, and/or mitigating pain in a subject, the method comprising administering to a subject a biomaterial comprising a therapeutically effective amount of adenosine, thereby reducing the subject's pain.

Disclosed herein is a method of treating, preventing, and/or mitigating pain in a subject having one or more bone fractures, the method comprising implanting a biomaterial comprising a therapeutically effective amount of adenosine at one or more bone fractures in a subject; wherein the subject's pain associated with the one or more bone fractures is reduced. Disclosed herein is a method of treating, preventing, and/or mitigating pain, the method comprising implanting in a subject having one or more bone fractures a biomaterial comprising a therapeutically effective amount of adenosine.

Disclosed herein is a method, the method comprising treating, preventing, and/or mitigating pain by administering to a subject having one or more bone fractures a biomaterial comprising a therapeutically effective amount of adenosine. Disclosed herein is a method, the method comprising treating, preventing, and/or mitigating pain by implanting in a subject having one or more bone fractures a biomaterial comprising a therapeutically effective amount of adenosine.

Disclosed herein is a method of providing non-opioid analgesia, the method comprising administering to a subject a biomaterial comprising a therapeutically effective amount of adenosine. Disclosed herein is a method of providing non-opioid analgesia, the method comprising administering to a subject a biomaterial comprising a therapeutically effective amount of adenosine; wherein the subject's pain is reduced. Disclosed herein is a method of providing non-opioid analgesia, the method comprising administering to a subject a biomaterial comprising a therapeutically effective amount of adenosine; thereby reducing the subject's pain. Disclosed herein is a method of providing non-opioid analgesia, the method comprising implanting in a subject in need thereof a biomaterial comprising a therapeutically effective amount of adenosine.

Disclosed herein is a method of providing non-opioid analgesia, the method comprising implanting in a subject in need thereof a biomaterial comprising a therapeutically effective amount of adenosine; wherein the subject's pain is reduced. Disclosed herein is a method of providing non-opioid analgesia, the method comprising implanting in a subject in need thereof a biomaterial comprising a therapeutically effective amount of adenosine; thereby reducing the subject's pain. Disclosed herein is a method, the method comprising providing non-opioid analgesia by administering to a subject in need thereof a biomaterial comprising a therapeutically effective amount of adenosine; thereby reducing the subject's pain.

Disclosed herein is a method, the method comprising providing non-opioid analgesia by implanting in a subject in need thereof a biomaterial comprising a therapeutically effective amount of adenosine; thereby reducing the subject's pain. Disclosed herein is a method of improving movement of a subject, the method comprising administering to a subject having pain a biomaterial comprising a therapeutically effective amount of adenosine, wherein the subject's pain associated is reduced, thereby allowing the subject to improve movement.

Disclosed herein is a method of improving movement of a subject, the method comprising administering to a subject having acute pain or chronic pain, post-surgical pain, cancer pain, injury pain, and/or pain associated with bone fracture a disclosed biomaterial, and reducing the subject's pain, thereby allowing the subject to improve movement. Disclosed herein is a method of improving movement in a subject, the method comprising improving a subject's movement by administering to the subject having pain a disclosed biomaterial, wherein the subject's pain is reduced. Disclosed herein is a method of improving movement in a subject, the method comprising reducing a subject's pain by administering to the subject having a pain a disclosed biomaterial, thereby improving movement in the subject. Disclosed herein is a method of improving movement in a subject, the method comprising administering to a subject a disclosed biomaterial, wherein the subject's pain is reduced, thereby allowing the subject to improve movement. Disclosed herein is a method of improving movement in a subject, the method comprising administering to a subject having pain a biomaterial comprising a therapeutically effective amount of adenosine, and reducing the subject's pain, thereby allowing the subject to improve movement. Disclosed herein is a method of improving movement in a subject, the method comprising administering to a subject having acute pain or chronic pain, post-surgical pain, cancer pain, injury pain, and/or pain associated with bone fracture a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject having pain a biomaterial comprising a therapeutically effective amount of adenosine, wherein the subject's pain is reduced. Disclosed herein is a method of improving movement in a subject, the method comprising reducing a subject's pain by administering to the subject having a pain a biomaterial comprising a therapeutically effective amount of adenosine, thereby improving movement in the subject. Disclosed herein is a method of improving movement in a subject, the method comprising administering to a subject a biomaterial comprising a therapeutically effective amount of adenosine, wherein the subject's pain is reduced, thereby allowing the subject to improve movement. Disclosed herein is a method of improving movement in a subject, the method comprising administering to a subject having one or more bone fractures a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject having one or more bone fractures a biomaterial comprising a therapeutically effective amount of adenosine, wherein the subject's pain is reduced. Disclosed herein is a method of improving movement in a subject, the method comprising reducing a subject's by administering to the subject having one or more bone fractures a biomaterial comprising a therapeutically effective amount of adenosine, thereby improving movement in the subject. Disclosed herein is a method of improving movement in a subject, the method comprising administering to a subject a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject a biomaterial comprising a therapeutically effective amount of adenosine, wherein the subject's pain is reduced. Disclosed herein is a method of improving movement in a subject, the method comprising reducing a subject's by administering to the subject a biomaterial comprising a therapeutically effective amount of adenosine, thereby improving movement in the subject.

Disclosed herein is a method, the method comprising improving a subject's movement by administering to a subject in need thereof a biomaterial comprising a therapeutically effective amount of adenosine; thereby reducing the subject's pain. Disclosed herein is a method, the method comprising improving a subject's movement by implanting in a subject in need thereof a biomaterial comprising a therapeutically effective amount of adenosine; thereby reducing the subject's pain. Disclosed herein is a method of making a disclosed biomaterial comprising adenosine.

### BRIEF DESCRIPTION OF THE FIGURES

FIG. 1 shows the characterization of polyethylene glycol diacrylate (PEGDA) by FTIR. Arrow at 1725 cm⁻¹ indicates the stretching frequency for the ester C═O bond in PEGDA. FTIR spectra were recorded using ZnSe crystal in attenuated total reflectance (ATR) mode.

FIG. 2 shows the characterization of polyethylene glycol diacrylate (PEGDA) by NMR. ¹HNMR spectrum of PEGDA recorded in CDCl₃ at 25° C.

FIG. 3 shows the characterization of N-acryloyl-6-aminocaproic acid (A6ACA) by FTIR. FTIR spectra were recorded using ZnSe crystal in attenuated total reflectance (ATR) mode. Arrows at 1655 cm⁻¹ and 1543 cm⁻¹ corresponding to the amide C—O and N—H stretching frequencies, respectively.

FIG. 4 shows the characterization of N-acryloyl-6-aminocaproic acid (A6ACA) by NMR. ¹HNMR spectrum of A6ACA recorded in D₂O at 25° C.

FIG. 5 shows the characterization of PEG-6ACA-PBA macroporous hydrogel. FTIR spectra of the lyophilized macroporous hydrogel recorded using ZnSe crystal in attenuated total reflectance (ATR) mode. Arrow at 1715 cm⁻¹ indicates the stretching frequency for the ester C—O bond of PEGDA in PEG-6ACA-PBA hydrogel.

FIG. 6 shows the characterization of PEG-6ACA-PBA macroporous hydrogel. ¹HNMR spectrum of the macroporous hydrogel recorded in D₂O at 25° C.

FIG. 7A-FIG. 7D show that fracture increased nociception in animals. FIG. 7A shows the percent static weight bearing of ipsilateral hindlimb before fracture (baseline) and 2 days post-fracture (dpf) of mice with fracture and sham control (mean±SEM, n=5 mice per group). FIG. 7B shows the relative nerve growth factor (Ngf) gene expression of whole bone with marrow compared between contralateral and ipsilateral tibiae of fractured mice at 3 dpf (mean±SEM, n=4 mice per group). FIG. 7C shows the immunofluorescence staining of Nissl-positive neuron cell bodies (red) and TRPV1 (green) in ipsilateral (fractured) and contralateral (nonfractured) L3-L4 DRG from injured mice at 3 days dpf. 4′,6-diamidino-2-phenylindole (DAPI) stained the nucleus (blue). Scale bar=200 μm. FIG. 7D shows quantified TRPV1 fluorescence (FL) intensity from the immunofluorescence images (mean±SEM, n>30 DRG neurons per mice, 5 mice per group). Statistical analyses were performed by Mann Whitney U test. *p<0.05, **p<0.01.

FIG. 8A-FIG. 8D show that DRG neurons exhibited high expression of adenosine A1 receptor. FIG. 8A shows the relative gene expression of adenosine receptors in unfractured L3-L4 DRG (mean±SEM, n=7 mice per group. Kruskal-Wallis with Dunn's post hoc test). Adora3 expression was undetectable. FIG. 8B shows the relative Adora1 gene expression of ipsilateral (fractured) and contralateral (nonfractured) L3-L4 DRG at 3 days post-fracture (dpf; mean±SEM, n=5 mice per group. Mann Whitney U test). FIG. 8C shows the immunofluorescence images of Nissl-positive neuron cell bodies (red) and ADORA1 (yellow) in ipsilateral and contralateral L3-L4 DRG from fractured mice at 3 dpf. DAPI stained the nucleus (blue). Scale bar, 200 μm. FIG. 8D shows the quantified mean ADORA1 fluorescence intensity (FL) from the immunofluorescence images (mean±SEM, n>20 neurons per mice, 5 mice per group. Mann Whitney U test). *p<0.05, **p<0.01.

FIG. 9A-FIG. 9H show adenosine mitigated DRG activity through adenosine A1 receptor. FIG. 9A shows immunofluorescence staining of TUBB3 (green) and TRPV1 (violet) in dissociated DRG neurons treated with or without nerve growth factor (NGF; 200 ng/mL). CTL: control. DAPI stained nucleus (blue). Scale bar, 200 μm. FIG. 9B shows quantified TRPV1 fluorescence intensity from the immunofluorescence images (mean±SEM, n>40 neurons per mice, 4 mice per group. Mann Whitney U test). FIG. 9C shows the experimental design of calcium imaging. 1 d: 1 day culture; ADO: adenosine (5 μM). Capsaicin (100 nM). FIG. 9D shows the normalized average signal intensity from Fura-2 calcium imaging. Treatments (T) of vehicle (Veh) or adenosine (ADO), and TRPV1 agonist capsaicin (Cap) was added at the specified time (black arrow). FIG. 9E shows the normalized peak intensity from Fura-2 calcium imaging (mean±SEM, n>10 cells per mice, 5 mice per group. Friedman test with Wilcoxon signed-rank test). FIG. 9F shows the experimental design used to examine the role of ADORA1 to inhibit the nociceptive function of dissociated DRG neurons. FIG. 9G shows the normalized average intensity of cultures treated with ADORA1 inhibitor DPCPX compared to the vehicle control (DMSO) using calcium imaging. Treatments (T) of vehicle (Veh) and DPCPX, and TRPV1 agonist capsaicin (Cap) was added at the specified time (black arrow). FIG. 9H shows the normalized peak intensity from the calcium imaging (mean±SEM, n>10 cells per mice, 5 mice per group. Mann Whitney U test). *p<0.05, **p<0.01, ***p<0.001.

FIG. 10A-FIG. 10D show the local delivery of adenosine improved weight bearing of injured limbs. FIG. 10A is a schematic illustrating the experimental timeline involving pin placement for stabilization, fracture surgery, biomaterial implantation, and weight bearing experiments. FIG. 10B shows the percentage of ipsilateral hindlimb weight bearing in fractured mice treated with control (CTL) or adenosine (ADO)-loaded macroporous hydrogels up to 8 days post-fracture (dpf; n=9 mice per group. (mean #SEM, n=9 mice per group. Friedman test with Wilcoxon signed-rank test was used to compare within the same treatment cohort over time). ^{§} p<0.05, ^{#}p<0.01. Mann Whitney U test was used to compare between CTL and ADO at the same timepoint. **p<0.01, ***p<0.001. FIG. 10C shows the effect size r of treatments on weight bearing corresponding to FIG. 10B. FIG. 10D shows the percent change of hindlimb weight bearing up to 8 dpf from pre-fracture (baseline) (mean±SEM, n=9 mice per group. Mann Whitney U test was used for each treatment group compared to baseline at each timepoint). *p<0.05, **p<0.01, ***p<0.001.

FIG. 11A-FIG. 11I show the local delivery of adenosine improved open field activity of fractured animals. FIG. 11A shows the experimental timeline of surgery, biomaterial implantation, and open field activity experiments. FIG. 11B shows the tracking of vertical activity (red dot) and horizontal activity (blue line) of fractured mice treated with control (CTL) or adenosine (ADO)-loaded macroporous hydrogels over a 5 min period at 7 days post-fracture (dpf). Each grid represents one mouse. FIG. 11C-FIG. 11H show the normalized ratio of total vertical activity count, vertical movement time, ambulatory activity count, ambulatory time, total distance, and rest time of ADO and CTL-treated mice at 7 dpf. Results are represented as a ratio of post-fracture divided by pre-fracture values (mean±SEM, n=9 mice per group. Mann Whitney U test was used for vertical activity count. Two-tailed unpaired t test was used for other parameters). *p<0.05, **p<0.01, ***p<0.001. FIG. 11I shows the effect size r and Cohen's d of treatment on open field activity for each parameter in FIG. 11C-FIG. 11H.

FIG. 12A-FIG. 12C show that local delivery of adenosine promoted fracture repair. FIG. 12A shows the 3D reconstructed (intact and cut) and radiographs from microCT imaging of fractured tibiae treated with adenosine (ADO) or control (CTL) at 21 dpf. FIG. 12B shows the quantification of total volume (TV) of regenerating calluses from microCT images (mean±SEM, n=5 mice per group. Mann Whitney U test). *p<0.05. White dashed box: callus region. FIG. 12C shows the safranin O staining of fractured tibiae treated with adenosine (ADO) or control (CTL) at 21 dpf. Scale bar=2 mm. Red arrow=cortical bridging. Black box=magnified region.

FIG. 13A-FIG. 13F show that osteoprogenitor cells exhibit high expression of adenosine A2B receptor. FIG. 13A shows the immunofluorescence staining of Td-Tomato-labeled LepR lineage cells (green) and ADORA2B (violet) in regenerating bone of fractured mice at 10 dpf and 21 dpf. DAPI stains nucleus (blue). dpf: days post-fracture. Scale bar=100 μm. Magnified image scale bar, 25 μm (n=3 mice). FIG. 13B shows the relative gene expression of adenosine receptors in bone marrow mesenchymal stromal cells (BM-MSCs; mean±SEM, n=3 mice pooled per group. Kruskal-Wallis test with Dunn's post hoc test). FIG. 13C-FIG. 13E show the gene expression of Runx2, Sp7, and Ibsp in BM-MSCs, respectively. Cells were treated with adenosine (ADO, 30 μg/mL) or control (CTL) in the presence of vehicle (DMSO) or ADORA2B inhibitor PSB 603 for 14 days. OM=osteogenic media. (mean±SEM, n=3 pooled mice per group. Friedman test with Wilcoxon signed-rank test). *p<0.05, **p<0.01, ***p<0.001. FIG. 13F shows the proposed action of adenosine on multiple cell populations and adenosine receptor subtypes resulting in reduced fracture pain and improved healing.

FIG. 14 shows representative images from calcium imaging using Fura-2 dye. TRPV1 agonist capsaicin was used to stimulate the cells and signals were normalized to the baseline (n>10 cells per mice, 5 mice per group). Arrows indicate cells with changing fluorescence after stimulation with TRPV1 agonist capsaicin.

FIG. 15A-FIG. 15B show that adenosine decreased functional activity of mouse DRG neurons. FIG. 15A shows a schematic of calcium imaging experiment. FIG. 15B shows the normalized peak intensity of dissociated DRG neurons in vitro treated with two different concentrations of adenosine (ADO) and stimulated by TRPV1 agonist capsaicin (mean±SEM, n>7 cells per group. Two-tailed unpaired t test). ***p<0.001.

FIG. 16A-FIG. 16C show that adenosine attenuated NGF-induced increase in membrane potential in DRG neurons. FIG. 16A shows the relative fluorescence intensity of membrane potential imaging of dissociated DRG neurons after 1 day of NGF treatment followed by adenosine treatment. Magnified views indicate cells with changing fluorescence intensity after stimulation with TRPV1 agonist capsaicin (Cap), which was added at the specified time (black arrow). FIG. 16B shows the normalized average signal intensity from membrane potential imaging. FIG. 16C shows the normalized peak intensity from membrane potential imaging (mean±SEM, n=7-13 cells per group. Two-way ANOVA with Tukey post hoc test). *p<0.05, **p<0.01.

FIG. 17 shows the in vitro release of adenosine. Cumulative percentage of in vitro release of adenosine from PEGDA-6ACA-PBA macroporous hydrogel over 21 days (n=3).

FIG. 18 shows the adenosine levels in circulation of treated mice. Concentration of adenosine in peripheral blood of fractured mice treated with control or adenosine-loaded macroporous hydrogel at 3 days post fracture (mean±SEM, n=3 mice. Mann Whitney U test).

FIG. 19A-FIG. 19F show that local delivery of adenosine improved open field activity of fractured animals. FIG. 19A shows normalized ratio of vertical activity count, FIG. 19B shows vertical movement time, FIG. 19C shows ambulatory activity count, FIG. 19D shows ambulatory time, FIG. 19E shows total distance, and FIG. 19F shows rest time of treated mice at 5-min intervals at 7 days post fracture. Results are normalized to the pre-fracture values (mean±SEM, n=9 mice per group. Mann Whitney U test). *p<0.05, **p<0.01, ***p<0.001.

FIG. 20A-FIG. 20B shows adenosine receptor gene expression of the whole bone marrow. FIG. 20A shows the relative gene expression of adenosine receptors in whole bone marrow of fractured mice (mean±SEM, n=3 mice per group. Kruskal-Wallis with Dunn's post hoc test was used for statistical analysis. FIG. 20B shows the relative gene expression of Adora2b of the bone marrow (BM) of contralateral and ipsilateral limbs (mean±SEM, n=3 mice per group. Mann Whitney U test). *p<0.05.

### DETAILED DESCRIPTION OF THE INVENTION

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered. In an aspect, a subject can be a human subject. In an aspect, a subject can have pain, be suspected of having pain, or be at risk of developing pain. In an aspect, a subject's pain can be due to one or more bone fractures. In an aspect, a subject's pain can be due to something other bone fractures or injuries.

As used herein, the term “diagnosed” means having been subjected to an examination by a person of skill, for example, a physician, and found to have a condition that can be diagnosed or treated by one or more of the disclosed biomaterials, disclosed pharmaceutical formulations, or a combination thereof, or by one or more of the disclosed methods. For example, “diagnosed with pain” or “diagnosed with a need for non-opioid analgesia” means having been subjected to an examination by a person of skill, for example, a physician, and found to have a condition that can be treated by one or more of the disclosed biomaterials, disclosed pharmaceutical formulations, or a combination thereof, or by one or more of the disclosed methods. For example, “suspected of having one or more bone fractures” can mean having been subjected to an examination by a person of skill, for example, a physician, and found to have a condition that can likely be treated by one or more of the disclosed biomaterials, disclosed pharmaceutical formulations, or a combination thereof, or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof.

A “patient” refers to a subject. In an aspect, a patient can refer to a subject that has been diagnosed with or is suspected of having one or more bone fractures. In an aspect, a patient can refer to a subject that has been diagnosed with or is suspected of having a one or more bone fractures and is seeking treatment or receiving treatment for the one or more bone fractures or pain associated with the one or more bone fractures.

As used herein, the phrase “identified to be in need of treatment for pain,” or the like, refers to selection of a subject based upon need for treatment of pain. For example, a subject can be identified as having a need for treatment of pain (e.g., due to one or more bone fractures) based upon an earlier diagnosis by a person of skill and thereafter subjected to treatment for pain (e.g., due to one or more bone fractures). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of a subject's pain (e.g., pain due to one or more bone fractures). This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having pain due to, for example, one or more bone fractures) or to the subject's pain level prior to the one or more bone fractures. Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the one or more bone fractures. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the one or more bone fractures. In an aspect, the inhibition or reduction can be 0-25%, 25-50%, 50-75%, or 75-100% as compared to native or control levels or to the subject's pain level prior to the one or more bone fractures. In an aspect, the subject's pain level prior to the onset of pain can be used as a control.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating, preventing, and/or mitigating pain—such as acute and chronic pain, post-surgical pain, injury pain, and/or pain associated with bone fracture—can reduce the severity of the subject's pain by 1%-100% as compared to a control (such as, for example, an individual not having pain or to the subject's level prior to the onset of acute or chronic paint, post-surgical pain, injury pain, and/or one or more bone fractures). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of pain. For example, treating a subject's pain can reduce one or more symptoms of pain in a subject by 1%-100% as compared to a control (such as, for example, an individual not having pain due to, for example, one or more bone fractures or to the subject's level prior to the one or more bone fractures). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of a subject's pain. It is understood that treatment does not necessarily refer to a cure or complete ablation or eradication of pain. However, in an aspect, treatment can refer to a cure or complete ablation or eradication of pain.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing pain or the worsening of pain is intended. The words “prevent” and “preventing” and “prevention” also refer to prophylactic or preventative measures for protecting or precluding a subject (e.g., an individual) not having pain or a given pain-related complication from progressing to that complication (such as, for example, chronic or debilitating pain).

As used herein, the terms “administering” and “administration” refer to any method of providing one or more of the disclosed compositions or disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-arterial administration, intramuscular administration, and subcutaneous administration. Administration of a disclosed biomaterial can be local, for example, at the site of one or more bone fractures. Administration can be continuous or intermittent.

In an aspect, the skilled person can determine an efficacious dose, an efficacious schedule, and an efficacious route of administration for one or more of the disclosed compositions, disclosed pharmaceutical formulations, or a combination thereof so as to treat or prevent pain (such as pain due to one or more bone fractures). In an aspect, the skilled person can also alter, change, or modify an aspect of an administering step to improve efficacy of one or more of the disclosed biomaterials, disclosed pharmaceutical formulations, or a combination thereof.

As used herein, “bone growth factor” or “osteoinductive growth factor” refers to a growth factor or other proteins that supports osteoinduction. Examples of such growth factors include, but are not limited to, bone morphogenic proteins (e.g., BMP-2, -4, -6, -7 or -9), basic fibroblast growth factor (bFGF), insulin-like growth factor-I and -II (IGF-I and IGF-II), platelet derived growth factor (PDGF), and transforming growth factor-betas (TGF-βs).

As used herein, “modifying the method” can comprise modifying or changing one or more features or aspects of one or more steps of a disclosed method. For example, in an aspect, a method can be altered by changing the amount of one or more of the disclosed biomaterials, disclosed pharmaceutical formulations, or a combination thereof administered to a subject, or by changing the frequency of administration of one or more of the disclosed biomaterials, disclosed pharmaceutical formulations, or a combination thereof to a subject, or by changing the duration of time the disclosed biomaterials, the disclosed pharmaceutical formulations, or a combination thereof are administered to a subject.

As used herein, “concurrently” means (1) simultaneously in time, or (2) at different times during the course of a common treatment schedule.

The term “contacting” as used herein refers to bringing one or more of the disclosed biomaterials, disclosed pharmaceutical formulations, or a combination thereof together with a target area or intended target area in such a manner that the one or more of the disclosed biomaterials, disclosed pharmaceutical formulations, or a combination thereof exert an effect on the intended target or targeted area either directly or indirectly. In an aspect, a target area or intended target area can be a subject's one or more bone fractures.

As used herein, “determining” can refer to measuring or ascertaining (subjectively, objectively, or both) the presence and severity of pain, such as, for example, pain due to one or more bone fractures. Methods and techniques used to determine the presence and/or severity of pain are typically known to the medical arts. For example, the art is familiar with the ways to identify and/or diagnose the presence, severity, or both of pain (such as, for example, pain due to one or more bone fractures).

As used herein, “effective amount” and “amount effective” can refer to an amount that is sufficient to achieve the desired result such as, for example, the treatment and/or prevention of pain (e.g., pain due one or more confirmed bone fractures or one or more suspected bone fractures). As used herein, the terms “effective amount” and “amount effective” can refer to an amount that is sufficient to achieve the desired effect on an undesired condition (e.g., pain, chronic pain, debilitating pain, etc.). The skilled person can determine an “effective amount” based on a number of factors.

For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed biomaterial, a disclosed pharmaceutical formulation, or a combination thereof that (i) treats the particular disease, condition, or disorder (e.g., pain), (ii) attenuates, ameliorates, or eliminates one or more symptoms of the particular disease, condition, or disorder (e.g., pain), or (iii) delays the onset of one or more symptoms of the particular disease, condition, or disorder described herein (e.g., pain). The specific therapeutically effective dose level for any particular patient will depend upon a variety of factors including the disorder being treated and the severity of the disorder; the biomaterials or disclosed pharmaceutical formulations employed; the disclosed methods employed; the age, body weight, general health, sex and diet of the patient; the time of administration; the route of administration; the rate of excretion of the disclosed biomaterials or disclosed pharmaceutical formulations employed; the duration of the treatment; drugs used in combination or coincidental with the disclosed biomaterials or disclosed pharmaceutical formulations employed, and other like factors well known in the medical arts. For example, it is well within the skill of the art to start doses of the disclosed biomaterials or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed biomaterials or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, pain (e.g., pain due to one or more bone fractures).

“Adenosine” can refer to a naturally occurring nucleoside found in the body. Adenosine also includes analogs, derivatives, and salts thereof. Extracellular adenosine (ADO) regulates tissue function by activating G-protein-coupled adenosine A1 receptor (A1R or ADORA1), A2A receptor (A2AR or ADORA2A), A2B receptor (A2BR or ADORA2B), and A3 receptor (A3R or ADORA3) (Jacobson K A, et al. (2006) Nat Rev Drug Discov. 5 (3): 247-264; Sheth S, et al. (2014) Int J Mol Sci. 15 (2): 2024-2052; Ciruela F, et al. (2010) Biochim Biophys Acta. 1798 (1): 9-20). Studies have demonstrated the key role played by extracellular ADO in bone health and bone tissue regeneration (Strazzulla L C, et al. (2016) Purinergic Signal. 12 (4): 583-593; Mediero A, et al. (2013) Trends Endocrinol Metab. 24 (6): 290-300; Ham J, et al. (2012) Front Endocrinol (Lausanne). 3:113; Lopez C D, et al. (2018) Adv Drug Deliv Rev. 2018; Rao V, et al. (2015). Front Bioeng Biotechnol. 3:185; Shih Y R, et al. (2014) Proc Natl Acad Sci USA. 111 (3): 990-995; Carroll S H, et al. (2013) Expert Rev Mol Med. 15:e1). ADO promotes osteogenic differentiation of stem cells including bone marrow derived mesenchymal progenitor cells (MSCs) (34, 35, 37, 38), and a decrease in ADO correlates with osteoporotic bone loss that can be rescued by increased A2BR activity (Shih Y V, et al. (2019) Sci Adv. 5 (8): eaax1387). Furthermore, the localization of ADO at the fracture site increased vascularization and accelerated fracture repair (Zeng Y, et al. (2020) Adv Mater. 32 (8):e1906022.). ADO has also been shown to provide analgesic effects on both peripheral and central nervous systems (PNS and CNS, respectively) (Sawynok J, et al. (2003) Prog Neurobiol. 69 (5): 313-340; Sowa N A, et al. (2010) J Neurosci. 30 (6): 2235-2244; Sawynok J. (2016) Neurosci. 338:1-18). The relief of pain by ADO has been shown to be achieved through its binding to A1R and A3R to suppress the activation of nociceptors or spinal microglia (Goldman N, et al. (2010) Nat Neurosci. 13 (7): 883-888; Kan H W, et al. (2018) Pain. 159 (8): 1580-1591; Coppi E, et al. (2019) Pain. 160 (5): 1103-1118; Terayama R, et al. (2018) Exp Brain Res. 236 (12): 3203-3213). A3 adenosine receptor agonist attenuates neuropathic pain by suppressing activation of microglia and conv. The effect of extracellular ADO on various neurons including olfactory bulb mitral cells, dorsal hippocampus, retinal ganglion cells, retrotrapezoid nucleus neurons, substantia nigra neurons, and dorsal root ganglion have been recognized (Rotermund N, et al. (2018) J Physiol. 2018; 596 (4): 717-733; Kim C S, et al. (2015) J Neurophysiol. 2015; 113 (7): 2511-2523; Clark B D, et al. (2009) J Neuroscience. 29 (36): 11237-11245; James S D, et al. (2018). Eneuro. 5 (6); Andoh T, et al. (2006) Brain Res. 1124 (1): 55-61; Sowa N A, et al. (2010) J Neurosci. 30 (31): 10282-10293).

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “chronic pain” refers to pain that lasts a long time. A popular alternative definition of chronic pain, involving no arbitrarily fixed durations is “pain that extends beyond the expected period of healing.” Chronic pain can be cancer pain or pain associated with an injury or pain due to cancer.

As used herein, “nociceptive pain” refers to pain caused by stimulation of peripheral nerve fibers that respond only to stimuli approaching or exceeding harmful intensity (nociceptors). Nociceptive pain can be classified according to the mode of noxious stimulation; the most common categories being “thermal” (heat or cold), “mechanical” (crushing, tearing, etc.), and “chemical” (iodine in a cut, chili powder in the eyes). A subset of nociceptive pain is called “inflammatory” pain, as it results from tissue damage and the response of innate inflammatory responses. Nociceptive pain can also be divided into “visceral,” “deep somatic”, and “superficial somatic” pain. Visceral structures are highly sensitive to stretch, ischemia, and inflammation, but relatively insensitive to other stimuli that normally evoke pain in other structures, such as burning and cutting. Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly localized pain. Examples include sprains and broken bones. Superficial pain is initiated by activation of nociceptors in the skin or other superficial tissue, and is sharp, well-defined, and clearly located. Examples of injuries that produce superficial somatic pain include minor wounds and minor (first degree) burns.

As used herein, “neuropathic pain” can refer pain caused by damage or disease that affects the somatosensory system. It may be associated with abnormal sensations called dysesthesia, and pain produced by normally non-painful stimuli (allodynia). Neuropathic pain may have continuous and/or episodic (paroxysmal) components. The latter are likened to an electric shock. Common qualities include burning or coldness, “pins and needles” sensations, numbness, and itching. Nociceptive pain, by contrast, is more commonly described as aching. Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain. Central neuropathic pain is found in spinal cord injury, multiple sclerosis, and some strokes. Aside from diabetes and other metabolic conditions, the common causes of painful peripheral neuropathies are herpes zoster infection, HIV-related neuropathies, nutritional deficiencies, toxins, remote manifestations of malignancies, immune mediated disorders, and physical trauma to a nerve trunk. Neuropathic pain is common in cancer as a direct result of cancer on peripheral nerves (e.g., compression by a tumor), or as a side effect of chemotherapy, radiation injury, or surgery. After a peripheral nerve lesion, aberrant regeneration may occur. Neurons become unusually sensitive and develop spontaneous pathological activity, abnormal excitability, and heightened sensitivity to chemical, thermal and mechanical stimuli. This phenomenon is called “peripheral sensitization.”

The (spinal cord) dorsal horn neurons give rise to the spinothalamic tract (STT), which constitutes the major ascending nociceptive pathway. As a consequence of ongoing spontaneous activity arising in the periphery, STT neurons develop increased background activity, enlarged receptive fields and increased responses to afferent impulses, including normally innocuous tactile stimuli. This phenomenon is called central sensitization. Central sensitization is an important mechanism of persistent neuropathic pain.

Other mechanisms, however, can take place at the central level after peripheral nerve damage. The loss of afferent signals can induce functional changes in dorsal horn neurons. A decrease in the large fiber input can decrease activity of interneurons inhibiting nociceptive neurons, i.e., loss of afferent inhibition. Hypoactivity of the descending anti-nociceptive systems or loss of descending inhibition can be another factor. With loss of neuronal input (deafferentation) the STT neurons can begin to fire spontaneously, a phenomenon designated “deafferentation hypersensitivity.” Neuroglia (“glial cells”) may play a role in central sensitization. Peripheral nerve injury can induce glia to release proinflammatory cytokines and glutamate—which, in turn influence neurons.

As used herein, the term “package insert” is used to refer to instructions customarily included in commercial packages of therapeutic products, that contain information about the indications, usage, dosage, administration, contraindications and/or warnings concerning the use of such therapeutic products (such as, for example, one or more disclosed biomaterials, one or more disclosed pharmaceutical formulations, and one or more disclosed therapeutic agents).

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed biomaterial comprising adenosine, a disclosed pharmaceutical formulation comprising a disclosed biomaterial, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more disclosed therapeutic agents) to a subject having or diagnosed with pain such as, for example, pain due to one or more bone fractures.

Disclosed are the components to be used to prepare the disclosed biomaterials or disclosed pharmaceutical formulations as well as the disclosed biomaterials or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, then specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Adenosine

Adenosine is a naturally occurring nucleoside, and extracellular adenosine regulates tissue function by activating the G-protein-coupled adenosine receptors—A1 receptor (ADORA1), A2A receptor (ADORA2A), A2B receptor (ADORA2B), and A3 receptor (ADORA3) (Jacobson K A, et al. (2006) Nat Rev Drug Discov. 5:247-264; Borea P A, et al. (2018) Physiol Rev. 98:1591-1625; Cronstein B N, et al. (2017) Nat Rev Rheumatol. 13:41-51). Adenosine has shown extensive analgesic effects on neuropathic and inflammatory pain in both the peripheral nervous system (PNS) and central nervous system (CNS) (Sawynok J. (2016) Neuroscience. 338:1-18). The relief of peripheral pain has been attributed through its binding to A1R and A3R on nociceptors or spinal microglia to suppress their activation (Goldman N, et al. (2010) Nat Neurosci. 13:883-888; Coppi E, et al. (2019) Pain. 160:1103-1118; Terayama R, et al. (2018) Exp Brain Res. 236:3203-3213).

Extracellular adenosine plays in bone health and regeneration (Mediero A, et al. (2013) Trends Endocrinol Metab. 24:290-300; Carroll S H, et al. (2013) Expert Rev Mol Med. 15:e1; Zeng Y, et al. (2020) Adv Mater. 32:e1906022), and studies have demonstrated impaired fracture healing in Cd73 (an enzyme that generates extracellular adenosine), Adora2a, and Adora2b knockout mice (Bradaschia-Correa V, et al. (2017) Tissue Cell. 49:545-551; Mediero A, et al. (2015) FASEB J. 29:1577-1590; Carroll S H, et al. (2012) J Biol Chem. 287:15718-15727). Localized administration of extracellular adenosine at the fracture site promoted vascularization and improved fracture repair in a murine tibial fracture model (Zeng Y, et al. (2020) Adv Mater. 32:e1906022). Similarly, osteoporotic mice treated with ADORA2B agonist displayed a significant reduction in bone loss (Shih Y B, et al. (2019) Sci Adv. 5:eaax1387). In addition to its role in bone repair, adenosine also exhibits analgesic effects and perturbation of adenosine signaling has been shown to play a role in inflammatory and neuropathic pain (Sawynok J (2016) Neuroscience. 338:1-18). Activation of ADORA1 and ADORA3 on nociceptors, and ADORA3 on spinal microglia have been shown to elicit analgesic effects (Goldman N, et al. (2010) Nat Neurosci. 13:883-888; Coppi E, et al. (2019) Pain. 160:1103-1118). Targeting pain at the origin of noxious stimuli by local delivery of analgesics could avoid side effects while providing pain relief (McDougall J J, (2011) Biochim Biophys Acta. 1812:459-467; White PF, (2017) Expert Opin Pharmacother. 18:329-333; Sehgal N, et al. (2011) Pain Physician. 14:249-258). As described below in the Examples, adenosine reduced the functional activity of dorsal root ganglion (DRG) neurons through ADORA1, and promoted osteogenic differentiation of mesenchymal stromal cells (MSCs) through ADORA2B. In mice with fracture injury, biomaterial-assisted local delivery of adenosine to the fracture site alleviated fracture/post-surgical pain while promoting bone healing.

However, despite a short half-life of <10 s in the circulation (Brink H L, et al. (2015) Pharmacotherapy. 35:1117-1123), systemic administration of adenosine is associated with undesirable cardiac side effects that limit its utility and route of administration (Zylka M J. (2011) Trends Mol Med. 17:188-196). While the in vitro effect of extracellular adenosine on various neurons including olfactory bulb mitral cells, dorsal hippocampus, retinal ganglion cells, retrotrapezoid nucleus neurons, substantia nigra neurons, and dorsal root ganglion (DRG) have been recognized, the clinical feasibility of its use as a drug to manage trauma pain has not been reported.

| Sequence Identifier | Protein/Species |
|---|---|
| SEQ ID NO: 25 | ADORA1/Human |
| SEQ ID NO: 26 | ADORA2A/Human |
| SEQ ID NO: 27 | ADORA2B/Human |
| SEQ ID NO: 28 | ADORA3/Human |
| SEQ ID NO: 29 | ADORA1/Mouse |
| SEQ ID NO: 30 | ADORA2A/Mouse |
| SEQ ID NO: 31 | ADORA2B/Mouse |
| SEQ ID NO: 32 | ADORA3/Mouse |

### C. Compositions

### 1. Biomaterials

Disclosed herein is a biomaterial comprising a therapeutically effective amount of adenosine for treating, preventing, and/or mitigating pain. In an aspect, pain can comprise both acute and chronic pain, post-surgical pain, cancer pain, injury pain, and pain associated with bone fracture. In an aspect, pain can comprise any type of pain or pain having any origin.

Disclosed herein is a biomaterial comprising macroporous scaffolds. Disclosed herein is a biomaterial comprising macroporous scaffolds, wherein the scaffolds comprise 3-acrylamido phenylboronic acid (3-APBA). Disclosed herein is a biomaterial comprising macroporous scaffolds, wherein the scaffolds comprise a hydrogel comprising 3-acrylamido phenylboronic acid (3-APBA). Disclosed herein is a biomaterial comprising macroporous scaffolds comprising adenosine. Disclosed herein is a biomaterial comprising macroporous scaffolds, wherein the scaffolds comprise 3-acrylamido phenylboronic acid (3-APBA) and adenosine. Disclosed herein is a biomaterial comprising macroporous scaffolds, wherein the scaffolds comprise a hydrogel comprises 3-acrylamido phenylboronic acid (3-APBA) and adenosine. Disclosed herein is a biomaterial comprising macroporous scaffolds, wherein the scaffolds comprise adenosine. Disclosed herein is a biomaterial comprising 3-APBA-conjugated macroporous scaffolds, wherein the scaffolds comprise adenosine. In an aspect, 3-APBA can be conjugated to the disclosed scaffolds. In an aspect, a disclosed biomaterial can comprise from about 0.10 mg to about 1.0 mg adenosine per 1 mg of the disclosed macroporous scaffolds. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous scaffolds. In an aspect, a disclosed biomaterial can comprise more than 1.0 mg adenosine per 1 mg of the disclosed macroporous scaffolds.

Disclosed herein is a biomaterial comprising a hydrogel. Disclosed herein is a biomaterial comprising a hydrogel, wherein the hydrogel comprises macroporous scaffolds. Disclosed herein is a biomaterial comprising a hydrogel, wherein the hydrogel comprises macroporous scaffolds comprising 3-acrylamido phenylboronic acid (3-APBA). Disclosed herein is a biomaterial comprising a hydrogel comprising adenosine. Disclosed herein is a biomaterial comprising a hydrogel, wherein the hydrogel comprises 3-acrylamido phenylboronic acid (3-APBA) and adenosine. Disclosed herein is a biomaterial comprising 3-APBA-conjugated hydrogel, wherein the hydrogel comprises macroporous scaffolds and adenosine. In an aspect, 3-APBA can be conjugated to the disclosed scaffolds. In an aspect, a disclosed biomaterial can comprise from about 0.10 mg to about 1.0 mg adenosine per 1 mg of the disclosed hydrogel. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed hydrogel. In an aspect, a disclosed biomaterial can comprise more than 1.0 mg adenosine per 1 mg of the disclosed hydrogel.

Disclosed herein is a biomaterial comprising poly(ethylene glycol)-co-6 aminocaproic acid macroporous scaffolds. Disclosed herein is a biomaterial comprising poly(ethylene glycol)-co-6 aminocaproic acid macroporous scaffolds, wherein the scaffolds comprise 3-acrylamido phenylboronic acid (3-APBA). Disclosed herein is a biomaterial comprising macroporous scaffolds, wherein the scaffolds comprise a poly(ethylene glycol)-co-6 aminocaproic acid hydrogel comprising 3-acrylamido phenylboronic acid (3-APBA). Disclosed herein is a biomaterial comprising poly(ethylene glycol)-co-6 aminocaproic acid macroporous scaffolds comprising adenosine. Disclosed herein is a biomaterial comprising poly(ethylene glycol)-co-6 aminocaproic acid macroporous scaffolds, wherein the scaffolds comprise 3-acrylamido phenylboronic acid (3-APBA) and adenosine. Disclosed herein is a biomaterial comprising macroporous scaffolds, wherein the scaffolds comprise a poly(ethylene glycol)-co-6 aminocaproic acid hydrogel comprises 3-acrylamido phenylboronic acid (3-APBA) and adenosine. Disclosed herein is a biomaterial comprising poly(ethylene glycol)-co-6 aminocaproic acid macroporous scaffolds, wherein the scaffolds comprise adenosine. Disclosed herein is a biomaterial comprising 3-APBA-conjugated poly(ethylene glycol)-co-6 aminocaproic acid macroporous scaffolds, wherein the scaffolds comprise adenosine. In an aspect, 3-APBA can be conjugated to the disclosed scaffolds.

Disclosed herein is a biomaterial comprising a poly(ethylene glycol)-co-6 aminocaproic acid hydrogel. Disclosed herein is a biomaterial comprising a poly(ethylene glycol)-co-6 aminocaproic acid hydrogel, wherein the hydrogel comprises macroporous scaffolds. Disclosed herein is a biomaterial comprising a poly(ethylene glycol)-co-6 aminocaproic acid hydrogel, wherein the hydrogel comprises macroporous scaffolds comprising 3-acrylamido phenylboronic acid (3-APBA). Disclosed herein is a biomaterial comprising a poly(ethylene glycol)-co-6 aminocaproic acid hydrogel comprising adenosine. Disclosed herein is a biomaterial comprising a poly(ethylene glycol)-co-6 aminocaproic acid hydrogel, wherein the hydrogel comprises 3-acrylamido phenylboronic acid (3-APBA) and adenosine. Disclosed herein is a biomaterial comprising poly(ethylene glycol)-co-6 aminocaproic acid hydrogel-3-APBA-conjugated, wherein the hydrogel comprises macroporous scaffolds and adenosine. In an aspect, 3-APBA can be conjugated to the disclosed scaffolds.

Disclosed herein is a biomaterial comprising a poly(ethylene glycol)-co-6 aminocaproic acid macroporous hydrogel, wherein the macroporous hydrogel are functionalized with 3-acrylamido phenylboronic acid (3-APBA). Disclosed herein is a biomaterial comprising a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine.

In an aspect, a disclosed poly(ethylene glycol) diacrylate (PEGDA) can be derived from or generated using poly(ethylene glycol) (PEG). In an aspect, a disclosed N-acryloyl-6-aminocaproic acid (A6ACA) can be derived from or generated using 6-aminocaproic acid (6ACA). In an aspect, a disclosed poly(ethylene glycol)-co-6 aminocaproic acid macroporous hydrogel can be derived from or generated using poly(ethylene glycol) (PEG) and using 6-aminocaproic acid (6ACA).

In an aspect, a disclosed biomaterial can comprise from about 0.10 mg to about 1.0 mg adenosine per 1 mg of the disclosed macroporous hydrogel. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous hydrogel. In an aspect, a disclosed biomaterial can comprise more than 1.0 mg adenosine per 1 mg of the disclosed macroporous hydrogel.

In an aspect, a disclosed biomaterial can comprise a therapeutically effective amount of adenosine. In an aspect, a disclosed hydrogel can comprise a therapeutically effective amount of adenosine. In an aspect, a therapeutically effective amount in a disclosed biomaterial and/or a disclosed hydrogel can be an amount effective for treating, preventing, and/or mitigating pain. In an aspect, a disclosed biomaterial can comprise from about 0.10 mg to about 1.0 mg adenosine per 1 mg of the disclosed biomaterial. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed biomaterial. In an aspect, a disclosed biomaterial can comprise more than 1.0 mg adenosine per 1 mg of the disclosed biomaterial.

Specific dosing regimens are within the purview of one of ordinary skill in the art. In an aspect, for example, a disclosed biomaterial can comprise from about 1 mg/kg to about 30 mg/kg of ADO to body weight. In an aspect, for example, a disclosed biomaterial can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed biomaterial can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect of a disclosed biomaterial, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect, a disclosed biomaterial can comprise a therapeutically effective amount of adenosine for treating, preventing, and/or mitigating pain. In an aspect, a disclosed hydrogel can comprise a therapeutically effective amount of adenosine for treating pain, preventing, and/or mitigating pain.

In an aspect, a disclosed biomaterial can comprise a polymer. In an aspect, a disclosed hydrogel can comprise a polymer. In an aspect, a disclosed biomaterial can comprise one or more disclosed therapeutic agents. In an aspect, a disclosed hydrogel can comprise one or more disclosed therapeutic agents. Therapeutic agents are known to the art and disclosed herein.

Disclosed herein is a biomaterial comprising a polymer, a bioactive molecule binding moiety, a bone targeting moiety, and a bioactive molecule. Disclosed herein is a biomaterial comprising a polymer, a bioactive molecule binding moiety, and a bioactive molecule. Disclosed herein is a biomaterial comprising a polymer and a bioactive molecule binding moiety. Disclosed herein is a biomaterial comprising a polymer and a bioactive molecule.

As used herein, “biomaterial” can refer to any material suitable for in vivo applications. In an aspect, a disclosed biomaterial can be used for a targeted or localized delivery of a therapeutic agent such as, for example, a small molecule therapeutic agent. In an aspect, a disclosed biomaterial can comprise a macroporous hydrogel, macroporous scaffolds, or a hydrogel comprising macroporous scaffolds. In an aspect, a disclosed biomaterial can be used for a targeted or localized delivery of a therapeutic agent such as, for example, adenosine. In an aspect, a disclosed biomaterial can comprise a macroporous hydrogel, macroporous scaffolds, or a hydrogel comprising macroporous scaffolds, wherein the disclosed biomaterial comprises a therapeutic agent such as, for example, adenosine. In an aspect, a disclosed biomaterial can be used for treating, preventing, and/or mitigating pain. In an aspect, a disclosed biomaterial can be used for a targeted or localized delivery of a therapeutic agent such as, for example, adenosine in a subject having pain. In an aspect, a disclosed biomaterial can comprise a macroporous hydrogel, macroporous scaffolds, or a hydrogel comprising macroporous scaffolds, wherein the disclosed biomaterial comprises a therapeutic agent such as, for example, adenosine.

In an aspect, a disclosed biomaterial can comprise a polymer and a disclosed polymer can be any biologically compatible polymer. A disclosed polymer can comprise a single type of monomer, or a disclosed polymer can comprise copolymers of two or more types of monomers. Any reference to a particular polymer herein is also intended to include a copolymer comprising the recited polymer. In an aspect, a polymer can comprise hyaluronic acid (HA), 2-(methacryloyloxy)ethyl acetoacetate (2MAEA), polyethylene glycol (PEG), polyethylene glycol diacrylate (PEGDA), or any combination thereof. In an aspect, a disclosed polymer can be HA or PEGDA. Polymers are known to the art and disclosed herein.

In an aspect, a disclosed polymer can be modified or adapted as appropriate with chemical moieties to assist with the conjugation of moieties or molecules of interest, or with the polymerization or formulation of the biomaterials as disclosed herein. Such modifications are well-known in the art.

In an aspect, a disclosed biomaterial can comprise a polymer functionalized with, or conjugated to, a bioactive molecule binding moiety. As used herein, “functionalized,” “functionalized with,” “conjugated,” and “conjugated to” can be used interchangeably to refer to the chemical coupling, typically though covalent binding, of two or more molecules. Molecules can, for example, be copolymerized, or a moiety may be included as a substituent to a particular functional group or molecule.

In an aspect, a disclosed biomaterial can be further functionalized with additional moieties and/or active agents that can be envisaged by one of skill in the art. The inclusion of any such additional moieties and/or agents would provide for the co-administration of these therapeutic agents with the bioactive molecules previously noted. The additional moieties and/or agents can also be included to assist in creating particular formats of the biomaterial. For example, DBCO and azide groups can be used as dopants to form a stable porous scaffold.

In an aspect, a disclosed “bioactive molecule” can refer to a therapeutic agent for the treatment of diseases, disorders, and conditions (e.g., bone fractures, bone loss, bone degeneration, etc.), and “bioactive molecule binding moiety” can refer to a moiety able to reversibly bind to, or to dynamically covalently bind, a bioactive molecule.

In an aspect, a disclosed bioactive molecule binding moiety can allow for a disclosed biomaterial to reversibly bind to, and therefore deliver, a disclosed bioactive molecule to a targeted or local site of interest (e.g., site of one or more bone fractures). The ability to reversibly bind a bioactive molecule can allow for the controlled or sustained release of a disclosed bioactive molecule at a site of interest (e.g., site of one or more bone fractures). In an aspect, where a disclosed biomaterial can be used to treat and/or prevent pain or bone degeneration and/or to promote bone regeneration, a disclosed bioactive molecule binding moiety can be an osteoanabolic molecule binding moiety, which is a moiety able to reversibly bind to, or to dynamically covalently bind, an osteoanabolic molecule.

In an aspect, a disclosed osteoanabolic molecule binding moiety can be a boronate molecule, which can form dynamic covalent bonds with cis-diol molecules such as adenosine. The boronate molecule can be, but is not limited to, phenylboronic acid (PBA) or 3-aminophenylboronic acid (3-APBA). In an aspect, a disclosed osteoanabolic molecule binding moiety can be a ketal group. As known to the art, ketal groups are pH sensitive and can support the on-demand release of adenosine, for example.

In an aspect, loading a disclosed biomaterial with adenosine (or another bioactive molecule of choice) can allow for the introduction of exogenous adenosine to a site in need of bone regeneration (e.g., one or more bone fractures) and/or minimization of bone degeneration, either by way of systemic delivery (where a bone targeting moiety is utilized) or by local administration of a disclosed biomaterial (where a bone targeting moiety is optionally utilized). Alternatively, a disclosed biomaterial can be administered locally (e.g., as a patch at the site of bone injury) without being loaded with adenosine. In an aspect, a disclosed biomaterial can be “loaded” in vivo with endogenous adenosine such that the disclosed biomaterial can be used to sequester adenosine at the site of bone injury or bone fracture. A disclosed biomaterial can leverage the innate adenosine surge after bone injury and sustains a localized adenosine signaling to accelerate tissue repair.

In an aspect, a disclosed biomaterial can comprise a bioactive molecule such as, for example, a disclosed therapeutic agent or an osteoanabolic molecule. As used herein, “osteoanabolic molecule” can refer to any molecule that helps increase bone mass, including but not limited to, Vitamin D, adenosine, teriparatide, strontium ranelate, and the like. In an aspect, such molecules can be “loaded” into a disclosed biomaterial (i.e., allowed to bind to the bioactive molecule/osteoanabolic molecule binding moiety) to enable a disclosed bioactive molecule to be administered for therapeutic use by way of a disclosed biomaterial.

In an aspect, a disclosed osteoanabolic molecule can be an adenosine A1 receptor (A1R or ADORA1) agonist, an adenosine A2A receptor (A2AR or ADORA2A) agonist, an adenosine A2B receptor (A2BR or ADORA2B) agonist, and A3 receptor (A3R or ADORA3) agonist, an adenosine compound, or a combination thereof. In an aspect, a disclosed adenosine compound can comprise adenosine or polyadenosine. In an aspect, a disclosed osteoanabolic molecule can comprise adenosine. Exemplary A1R or ADORA1 agonists include A1R or ADORA1 partial agonists. Exemplary A1R or ADORA1 agonists include but are not limited to 2-chloro-N6-cyclopentyladenosine, N6-cyclopentyladenosine, (±)-5′-chloro-5′-deoxy-ENBA, 2′-MeCCPA, N6-cyclohexyladenosine, tecadenoson, selodenoson, and SDZ WAG 994. A2BR agonists include A2BR partial agonists. Exemplary A2BR agonists include, but are not limited to, BAY 60-6583, NECA (N-ethylcarboxamidoadenosine), (S)-PHPNECA, LUF-5835, and LUF-5845. Exemplary A3R agonists include, but are not limited to, IB-MECA, 2-Cl-IB-MECA, HEMADO, and MRS 5698. The adenosine compound can be adenosine, polyadenosine, or an analog or derivative of adenosine. In an aspect, the use of a disclosed biomaterial can mitigate the short half-life and off-target effects of adenosine when administered without being complexed to the biomaterial.

In an aspect, a disclosed biomaterial can comprise a bone targeting moiety. In an aspect, a bone targeting moiety can comprise a bisphosphonate molecule. Inclusion of a bone targeting moiety can allow for the targeted delivery of a disclosed biomaterial to bone by systemic administration (vs. local administration), thereby avoiding or diminishing off-target effects of the osteoanabolic molecule that might otherwise occur by way of systemic administration of the osteoanabolic molecule. A disclosed bone targeting moiety can allow for the accumulation of the biomaterial in bone, including, e.g., the site of bone injury such as a bone fracture. A disclosed bone targeting moiety can be any apatite, hydroxyapatite, or bone binding agent such as an aptamer, peptide, small molecule, etc. In an aspect, a disclosed bone targeting moiety can be a bisphosphonate molecule. Exemplary bisphosphonate molecules include, but are not limited to, etidronate, clodronate, tiludronate, pamidronate, neridronate, olpadronate, alendronate, ibandronate, risedronate, and zoledronate. In certain embodiments, the bisphosphonate molecule is alendronate. A disclosed bone targeting moiety can be coupled to the polymer via amine coupling or any other suitable method.

In an aspect, a disclosed biomaterial can be formulated for systemic delivery, for local delivery, or both. In an aspect, a disclosed biomaterial can be formulated as a hydrogel, a nanogel, a microgel, a tablet, a patch, a coating for an orthopedic implant, an ointment, a cream, or a scaffold. In an aspect, a disclosed biomaterial can comprise a microgel having a diameter for example of 1-200 μm. For example, in an aspect, a disclosed biomaterial can comprise a macroporous hydrogel, macroporous scaffolds, or a hydrogel comprising macroporous scaffolds.

Preparation of the disclosed biomaterials can be by any method known in the art or disclosed herein, for example by way of emulsion photopolymerization, the use of microfluidics, etc. For example, biomaterials disclosed herein can be formulated in different forms for use in different applications. Such forms include, but are not limited to, gels (hydrogels, nanogels, microgels), tablets, patches, transdermal patches or devices, pouches, devices, coatings for orthopedic implants, ointments, creams, and scaffolds (including macroporous scaffolds).

Disclosed biomaterials can be administered systemically (e.g., intravenous or intraperitoneal) or locally (e.g., implantation or injection at site of defect) and can be degradable.

One of skill in the art will understand that particular forms may be best suited to particular applications, goals, and routes of administration. In non-limiting examples, nanogels and microgels are suitable for systemic (intravenous, intraperitoneal, etc.) administration, where the osteoanabolic molecule can delivered to bone tissue through targeting by the bone targeting moiety for the treatment a bone fracture, for example. The nanogels and microgels can also be used as building blocks to create injectable 3D scaffolds for local delivery. Here, a disclosed biomaterial can be functionalized with clickable units to allow for the formation of a porous space filling scaffold, which can be used for orthopedic injuries with space, such as tumor excised space, etc. In an aspect, tablets can be suitable for systemic (oral) administration, patches for local administration (e.g., at the site of a bone fracture), and creams or ointments, as well as transdermal patches or devices, for transdermal delivery.

In some embodiments, a disclosed biomaterial can be formulated as a pouch or device to be used as a surgically-implanted replenishable device, where the level of the osteoanabolic agent in the pouch or device can be re-loaded as needed noninvasively through local injection (e.g., injection of adenosine into the biomaterial pouch). In an aspect, the pouch or device can sequester endogenous adenosine.

In an aspect, disclosed hydrogels, nanogels, and microgels can be spherical in shape with a diameter in the range of 0.01 to 500 μm. In setting forth this range, it is intended that any range within the stated range, or any specific value falling within the range, be included even if not specifically enumerated. Accordingly, the nanogels and microgels can have a diameter of 0.1 μm-400 μm, of 1 μm-200 μm, of 10 μm-200 μm, of 60 nm-100 nm, of 90 nm-110 nm, of about 0.1 μm, of about 1 μm, of about 100 μm, of about 200 μm, etc.

In an aspect, a disclosed biomaterial can comprise one or more disclosed therapeutic agents. Therapeutic agents are known to the art and are disclosed herein. In an aspect, a disclosed therapeutic agent can comprise other osteoanabolic molecules and bone growth/bone healing promoting compounds.

In an aspect, a disclosed therapeutic agent can comprise a “biologically active agent” or “biologic active agent” or “bioactive agent” that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the disclosed compositions can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

In an aspect, a disclosed therapeutic agent can comprise “pharmaceutically active agent” and can include a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an anti-angina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; anti-inflammatory agents, including anti-asthmatic anti-inflammatory agents, anti-arthritis anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; anti-angina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. As used herein, the recitation of an anti-bacterial agent inherently encompasses the pharmaceutically acceptable salts thereof.

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents.

As used herein, the recitation of an anti-fungal agent inherently encompasses the pharmaceutically acceptable salts thereof.

Anti-viral agents are known to the art. As used herein, the recitation of any anti-viral agent inherently encompasses the pharmaceutically acceptable salts thereof.

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes). As used herein, the recitation of a corticosteroid inherently encompasses the pharmaceutically acceptable salts thereof.

Analgesics are well known in the art and include opioid, narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof.

The term “immunostimulant” is used herein to describe a substance which evokes, increases, and/or prolongs an immune response to an antigen. Immunomodulatory agents modulate the immune system, and, as used herein, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators. As used herein, the recitation of an immunostimulant inherently encompasses the pharmaceutically acceptable salts.

As used herein, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. As used herein, the recitation of an immune-based product inherently encompasses the pharmaceutically acceptable salts thereof.

### 2. Pharmaceutical Compositions

Disclosed herein is a pharmaceutical composition comprising a disclosed biomaterial. Disclosed herein is a pharmaceutical composition comprising a disclosed biomaterial and one or more therapeutic agents. Disclosed herein is a pharmaceutical composition comprising a disclosed hydrogel. Disclosed herein is a pharmaceutical composition comprising a disclosed hydrogel and one or more therapeutic agents.

Disclosed herein is a pharmaceutical composition comprising a disclosed biomaterial comprising a therapeutically effective amount of adenosine. Disclosed herein is a pharmaceutical composition comprising a disclosed biomaterial comprising a therapeutically effective amount of adenosine and one or more therapeutic agents. Disclosed herein is a pharmaceutical composition comprising a disclosed hydrogel comprising a therapeutically effective amount of adenosine. Disclosed herein is a pharmaceutical composition comprising a disclosed hydrogel comprising a therapeutically effective amount of adenosine and one or more therapeutic agents.

In an aspect of a disclosed pharmaceutical composition, a disclosed biomaterial can comprise from about 0.10 mg to about 1.0 mg adenosine per 1 mg of the disclosed macroporous scaffolds. In an aspect of a disclosed pharmaceutical composition, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous scaffolds. In an aspect of a disclosed pharmaceutical composition, a disclosed biomaterial can comprise more than 1.0 mg adenosine per 1 mg of the disclosed macroporous scaffolds.

Disclosed herein is a pharmaceutical composition comprising a biomaterial comprising poly(ethylene glycol)-co-6 aminocaproic acid macroporous scaffolds. Disclosed herein is a pharmaceutical composition comprising a biomaterial comprising poly(ethylene glycol)-co-6 aminocaproic acid macroporous scaffolds, wherein the scaffolds comprise 3-acrylamido phenylboronic acid (3-APBA). Disclosed herein is a pharmaceutical composition comprising a biomaterial comprising macroporous scaffolds, wherein the scaffolds comprise a poly(ethylene glycol)-co-6 aminocaproic acid hydrogel comprising 3-acrylamido phenylboronic acid (3-APBA). Disclosed herein is a pharmaceutical composition comprising a biomaterial comprising poly(ethylene glycol)-co-6 aminocaproic acid macroporous scaffolds comprising adenosine. Disclosed herein is a pharmaceutical composition comprising a biomaterial comprising poly(ethylene glycol)-co-6 aminocaproic acid macroporous scaffolds, wherein the scaffolds comprise 3-acrylamido phenylboronic acid (3-APBA) and adenosine. Disclosed herein is a pharmaceutical composition comprising a biomaterial comprising macroporous scaffolds, wherein the scaffolds comprise a poly(ethylene glycol)-co-6 aminocaproic acid hydrogel comprises 3-acrylamido phenylboronic acid (3-APBA) and adenosine. Disclosed herein is a pharmaceutical composition comprising a biomaterial comprising poly(ethylene glycol)-co-6 aminocaproic acid macroporous scaffolds, wherein the scaffolds comprise adenosine. Disclosed herein is a pharmaceutical composition comprising a biomaterial comprising 3-APBA-conjugated poly(ethylene glycol)-co-6 aminocaproic acid macroporous scaffolds, wherein the scaffolds comprise adenosine. In an aspect, 3-APBA can be conjugated to the disclosed scaffolds.

Disclosed herein is a pharmaceutical composition comprising a biomaterial comprising a poly(ethylene glycol)-co-6 aminocaproic acid hydrogel. Disclosed herein is a pharmaceutical composition comprising a biomaterial comprising a poly(ethylene glycol)-co-6 aminocaproic acid hydrogel, wherein the hydrogel comprises macroporous scaffolds. Disclosed herein is a pharmaceutical composition comprising a biomaterial comprising a poly(ethylene glycol)-co-6 aminocaproic acid hydrogel, wherein the hydrogel comprises macroporous scaffolds comprising 3-acrylamido phenylboronic acid (3-APBA). Disclosed herein is a pharmaceutical composition comprising a biomaterial comprising a poly(ethylene glycol)-co-6 aminocaproic acid hydrogel comprising adenosine. Disclosed herein is a pharmaceutical composition comprising a biomaterial comprising a poly(ethylene glycol)-co-6 aminocaproic acid hydrogel, wherein the hydrogel comprises 3-acrylamido phenylboronic acid (3-APBA) and adenosine. Disclosed herein is a pharmaceutical composition comprising a biomaterial comprising poly(ethylene glycol)-co-6 aminocaproic acid hydrogel-3-APBA-conjugated, wherein the hydrogel comprises macroporous scaffolds and adenosine. In an aspect, 3-APBA can be conjugated to the disclosed scaffolds.

Disclosed herein is a pharmaceutical composition comprising a biomaterial comprising a poly(ethylene glycol)-co-6 aminocaproic acid macroporous hydrogel, wherein the macroporous hydrogel are functionalized with 3-acrylamido phenylboronic acid (3-APBA).

Disclosed herein is a pharmaceutical composition comprising a biomaterial comprising a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine.

In an aspect, a disclosed poly(ethylene glycol) diacrylate (PEGDA) can be derived from or generated using poly(ethylene glycol) (PEG).

In an aspect, a disclosed N-acryloyl-6-aminocaproic acid (A6ACA) can be derived from or generated using 6-aminocaproic acid (6ACA).

In an aspect, a disclosed poly(ethylene glycol)-co-6 aminocaproic acid macroporous hydrogel can be derived from or generated using poly(ethylene glycol) (PEG) and using 6-aminocaproic acid (6ACA). In an aspect, a disclosed pharmaceutical composition comprising a disclosed biomaterial can comprise from about 0.10 mg to about 1.0 mg adenosine per 1 mg of the disclosed macroporous hydrogel. In an aspect, a disclosed pharmaceutical composition comprising a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous hydrogel. In an aspect, a disclosed pharmaceutical composition comprising a disclosed biomaterial can comprise more than 1.0 mg adenosine per 1 mg of the disclosed macroporous hydrogel.

In an aspect, a disclosed pharmaceutical composition comprising a biomaterial can be administered to a subject, either alone or in combination with a pharmaceutically acceptable excipient and/or carrier, in an amount sufficient to induce an appropriate biological response (e.g., increasing bone mass, reducing pain associated with one or more bone fractures).

In an aspect, an effective amount of a disclosed pharmaceutical composition can be given in one dose but is not restricted to one dose. In an aspect, administration can comprise multiple doses such as, for example 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90, 91-100, or over 100 doses of a disclosed biomaterial or a disclosed pharmaceutical composition. When more than one dose is administered, doses can be spaced by time intervals of 1 minute, 2 minutes, 3 minutes, 4 minutes, 5 minutes, 6 minutes, 7 minutes, 8 minutes, 9 minutes, 10 minutes, or more minutes, by intervals of about 1, 2, 3, 4, 5, 6, 7, 8, 9, 10, 11, 12, 13, 14, 15, 16, 17, 18, 19, 20, 21, 22, 23, 24 hours, and so on. In the context of hours, the term “about” means plus or minus any time interval within 30 minutes. The disclosed doses can also be spaced by time intervals of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, and combinations thereof. In an aspect of a disclosed method, doses are not required to be spaced equally apart in time, but can encompass non-equal intervals, such as a priming schedule consisting of administration at 1 day, 4 days, 7 days, and 25 days, just to provide a non-limiting example.

In an aspect, a dosing schedule for a disclosed method can be once/week, twice/week, three times/week, four times/week, five times/week, six times/week, seven times/week, once every two weeks, once every three weeks, once every four weeks, once every five weeks, six weeks, two months, three months, four months, five months, six months, seven months, eight months, nine months, ten months, eleven months, and twelve months. In an aspect, a dosing schedule for a disclosed method can be determined by one of skill in the art.

In an aspect, a dosing cycle can be repeated. In an aspect, a dosing cycle can be repeated about every 7 days, every 14 days, every 21 days, every 28 days, every 35 days, 42 days, every 49 days, every 56 days, every 63 days, every 70 days, and the like. In an aspect, an interval of non-dosing can occur between a cycle, where the interval can be about 7 days, 14 days, 21 days, 28 days, 35 days, 42 days, 49 days, 56 days, 63 days, 70 days, and the like. In an aspect, the term “about” means plus or minus one day, plus or minus two days, plus or minus three days, plus or minus four days, plus or minus five days, plus or minus six days, or plus or minus seven days.

Specific dosing regimens are within the purview of one of ordinary skill in the art. In an aspect, for example, a disclosed pharmaceutical composition can comprise from about 1 mg/kg to about 30 mg/kg of ADO to body weight. In an aspect, for example, a disclosed pharmaceutical composition can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed pharmaceutical composition can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect of a disclosed pharmaceutical composition, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect, a disclosed pharmaceutical composition can be administered to a subject having pain. In an aspect, pain can comprise both acute and chronic pain, post-surgical pain, cancer pain, injury pain, and/or pain associated with bone fracture. In an aspect, pain can comprise any type of pain or pain having any origin.

### 3. Kits

Disclosed herein is a kit comprising one or more components and/or reagents for use in a disclosed method of treating, preventing, and/or mitigating pain. Disclosed herein is a kit comprising one or more components and/or reagents for use in a disclosed method of improving movement. Disclosed herein is a kit comprising one or more components and/or reagents for use in a disclosed method of promoting healing. Disclosed herein is a kit comprising a disclosed biomaterial for administration to a subject. Disclosed herein is a kit comprising a disclosed pharmaceutical formulation for administration to a subject. Disclosed herein is a kit comprising a disclosed biomaterial and a disclosed pharmaceutical formulation for administration to a subject.

Disclosed herein is a kit comprising one or more components and/or reagents comprising adenosine for use in a disclosed method of treating, preventing, and/or mitigating pain. Disclosed herein is a kit comprising one or more components and/or reagents comprising adenosine for use in a disclosed method of improving movement. Disclosed herein is a kit comprising one or more components and/or reagents comprising adenosine for use in a disclosed method of promoting healing. Disclosed herein is a kit comprising a disclosed biomaterial for administration to a subject. Disclosed herein is a kit comprising a disclosed pharmaceutical formulation for administration to a subject. Disclosed herein is a kit comprising a disclosed biomaterial and a disclosed pharmaceutical formulation for administration to a subject. Disclosed herein is a kit comprising a disclosed biomaterial comprising a therapeutically effective amount of adenosine for treating, preventing, and/or mitigating pain.

In an aspect, a disclosed kit can comprise a biomaterial comprising poly(ethylene glycol)-co-6 aminocaproic acid macroporous scaffolds. In an aspect, a disclosed kit can comprise a biomaterial comprising poly(ethylene glycol)-co-6 aminocaproic acid macroporous scaffolds, wherein the scaffolds comprise 3-acrylamido phenylboronic acid (3-APBA). In an aspect, a disclosed kit can comprise a biomaterial comprising macroporous scaffolds, wherein the scaffolds comprise a poly(ethylene glycol)-co-6 aminocaproic acid hydrogel comprising 3-acrylamido phenylboronic acid (3-APBA). In an aspect, a disclosed kit can comprise a biomaterial comprising poly(ethylene glycol)-co-6 aminocaproic acid macroporous scaffolds comprising adenosine. In an aspect, a disclosed kit can comprise a biomaterial comprising poly(ethylene glycol)-co-6 aminocaproic acid macroporous scaffolds, wherein the scaffolds comprise 3-acrylamido phenylboronic acid (3-APBA) and adenosine. In an aspect, a disclosed kit can comprise macroporous scaffolds, wherein the scaffolds comprise a poly(ethylene glycol)-co-6 aminocaproic acid hydrogel comprises 3-acrylamido phenylboronic acid (3-APBA) and adenosine. In an aspect, a disclosed kit can comprise a biomaterial comprising poly(ethylene glycol)-co-6 aminocaproic acid macroporous scaffolds, wherein the scaffolds comprise adenosine. In an aspect, a disclosed kit can comprise a biomaterial comprising 3-APBA-conjugated poly(ethylene glycol)-co-6 aminocaproic acid macroporous scaffolds, wherein the scaffolds comprise adenosine. In an aspect, a disclosed kit can comprise a biomaterial comprising a poly(ethylene glycol)-co-6 aminocaproic acid hydrogel.

In an aspect, a disclosed kit can comprise a biomaterial comprising a poly(ethylene glycol)-co-6 aminocaproic acid hydrogel, wherein the hydrogel comprises macroporous scaffolds. In an aspect, a disclosed kit can comprise a biomaterial comprising a poly(ethylene glycol)-co-6 aminocaproic acid hydrogel, wherein the hydrogel comprises macroporous scaffolds comprising 3-acrylamido phenylboronic acid (3-APBA). In an aspect, a disclosed kit can comprise a biomaterial comprising a poly(ethylene glycol)-co-6 aminocaproic acid hydrogel comprising adenosine. In an aspect, a disclosed kit can comprise a biomaterial comprising a poly(ethylene glycol)-co-6 aminocaproic acid hydrogel, wherein the hydrogel comprises 3-acrylamido phenylboronic acid (3-APBA) and adenosine. In an aspect, a disclosed kit can comprise a biomaterial comprising poly(ethylene glycol)-co-6 aminocaproic acid hydrogel-3-APBA-conjugated, wherein the hydrogel comprises macroporous scaffolds and adenosine In an aspect, a disclosed kit can comprise a biomaterial comprising a poly(ethylene glycol)-co-6 aminocaproic acid macroporous hydrogel, wherein the macroporous hydrogel are functionalized with 3-acrylamido phenylboronic acid (3-APBA). In an aspect, a disclosed kit can comprise a biomaterial comprising a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine. In an aspect, a disclosed kit can comprise a biomaterial comprising macroporous scaffolds.

Disclosed herein is a biomaterial comprising macroporous scaffolds, wherein the scaffolds comprise 3-acrylamido phenylboronic acid (3-APBA). In an aspect, a disclosed kit can comprise a biomaterial comprising macroporous scaffolds, wherein the scaffolds comprise a hydrogel comprising 3-acrylamido phenylboronic acid (3-APBA). In an aspect, a disclosed kit can comprise a biomaterial comprising macroporous scaffolds comprising adenosine. In an aspect, a disclosed kit can comprise a biomaterial comprising macroporous scaffolds, wherein the scaffolds comprise 3-acrylamido phenylboronic acid (3-APBA) and adenosine. In an aspect, a disclosed kit can comprise a biomaterial comprising macroporous scaffolds, wherein the scaffolds comprise a hydrogel comprises 3-acrylamido phenylboronic acid (3-APBA) and adenosine. In an aspect, a disclosed kit can comprise a biomaterial comprising macroporous scaffolds, wherein the scaffolds comprise adenosine. In an aspect, a disclosed kit can comprise a biomaterial comprising 3-APBA-conjugated macroporous scaffolds, wherein the scaffolds comprise adenosine. In an aspect, 3-APBA can be conjugated to the disclosed scaffolds. In an aspect, a disclosed kit can comprise a biomaterial comprising a hydrogel. In an aspect, a disclosed kit can comprise a biomaterial comprising a hydrogel, wherein the hydrogel comprises macroporous scaffolds.

Disclosed herein is a biomaterial comprising a hydrogel, wherein the hydrogel comprises macroporous scaffolds comprising 3-acrylamido phenylboronic acid (3-APBA). In an aspect, a disclosed kit can comprise a biomaterial comprising a hydrogel comprising adenosine. In an aspect, a disclosed kit can comprise a biomaterial comprising a hydrogel, wherein the hydrogel comprises 3-acrylamido phenylboronic acid (3-APBA) and adenosine. In an aspect, a disclosed kit can comprise a biomaterial comprising 3-APBA-conjugated hydrogel, wherein the hydrogel comprises macroporous scaffolds and adenosine. In an aspect of a disclosed kit, a disclosed biomaterial can comprise from about 0.10 mg to about 1.0 mg adenosine per 1 mg of the disclosed macroporous scaffolds. In an aspect of a disclosed kit, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous scaffolds. In an aspect of a disclosed kit, a disclosed biomaterial can comprise more than 1.0 mg adenosine per 1 mg of the disclosed macroporous scaffolds.

In an aspect, for example, a disclosed kit can comprise from about 1 mg/kg to about 30 mg/kg of ADO to body weight. In an aspect, for example, a disclosed kit can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed kit can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect of a disclosed kit, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect, a disclosed kit can comprise multiple doses of a disclosed biomaterial, a disclosed pharmaceutical formulation, or a combination thereof. In an aspect, a disclosed kit can comprise at least two components constituting the kit. Together, the components constitute a functional unit for a given purpose (such as, for example, treating pain in a subject having one or more bone fractures). Individual member components may be physically packaged together or separately. For example, a disclosed kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed biomaterial, a disclosed pharmaceutical formulation, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed biomaterial, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed biomaterial, a disclosed pharmaceutical formulation, or a combination thereof can be used for treating, preventing, inhibiting, and/or ameliorating pain associated with one or more bone fractures or complications and/or symptoms associated with pain due to one or more bone fractures. A kit can comprise additional components necessary for administration such as, for example, other buffers, diluents, filters, needles, and syringes. In an aspect, a disclosed kit can comprise multiple doses of a disclosed biomaterial, a disclosed pharmaceutical formulation, or both.

### D. Methods

1. Methods of Treating, Preventing, and/or Mitigating Pain

Disclosed herein is a method of treating, preventing, and/or mitigating pain in a subject, the method comprising administering to a subject a biomaterial comprising a therapeutically effective amount of adenosine. Disclosed herein is a method of treating, preventing, and/or mitigating pain in a subject, the method comprising administering to a subject a biomaterial comprising a therapeutically effective amount of adenosine, wherein the subject's pain is reduced. Disclosed herein is a method of treating, preventing, and/or mitigating pain in a subject, the method comprising administering to a subject a biomaterial comprising a therapeutically effective amount of adenosine, thereby reducing the subject's pain. Disclosed herein is a method of treating, preventing, and/or mitigating pain in a subject having one or more bone fractures, the method comprising implanting a biomaterial comprising a therapeutically effective amount of adenosine at one or more bone fractures in a subject; wherein the subject's pain associated with the one or more bone fractures is reduced. Disclosed herein is a method of treating, preventing, and/or mitigating pain, the method comprising implanting in a subject having one or more bone fractures a biomaterial comprising a therapeutically effective amount of adenosine. Disclosed herein is a method, the method comprising treating, preventing, and/or mitigating pain by administering to a subject having one or more bone fractures a biomaterial comprising a therapeutically effective amount of adenosine. Disclosed herein is a method, the method comprising treating, preventing, and/or mitigating pain by implanting in a subject having one or more bone fractures a biomaterial comprising a therapeutically effective amount of adenosine.

In an aspect, a disclosed method of treating, preventing, and/or mitigating pain can comprise providing non-opioid analgesia.

In an aspect of a disclosed method, a disclosed biomaterial can comprise a macroporous hydrogel, macroporous scaffolds, or a hydrogel comprising macroporous scaffolds. In an aspect of a disclosed method, a disclosed biomaterial can comprise a hydrogel or macroporous scaffolds. In an aspect of a disclosed method, macroporous scaffolds can comprise 3-APBA.

In an aspect of a disclosed method, a disclosed biomaterial can comprise poly(ethylene glycol)-co-6 aminocaproic acid macroporous scaffolds. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA). In an aspect of a disclosed method, a disclosed biomaterial can comprise macroporous scaffolds, wherein the scaffolds comprise a poly(ethylene glycol)-co-6 aminocaproic acid hydrogel comprising 3-acrylamido phenylboronic acid (3-APBA). In an aspect of a disclosed method, a disclosed biomaterial can comprise poly(ethylene glycol)-co-6 aminocaproic acid macroporous scaffolds comprising adenosine. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA) and adenosine. In an aspect of a disclosed method, a disclosed biomaterial can comprise macroporous scaffolds, wherein the scaffolds comprise a poly(ethylene glycol)-co-6 aminocaproic acid hydrogel comprises 3-acrylamido phenylboronic acid (3-APBA) and adenosine. In an aspect of a disclosed method, a disclosed biomaterial can comprise poly(ethylene glycol)-co-6 aminocaproic acid macroporous scaffolds, wherein the scaffolds comprise adenosine. In an aspect of a disclosed method, a disclosed biomaterial can comprise comprising 3-APBA-conjugated poly(ethylene glycol)-co-6 aminocaproic acid macroporous scaffolds, wherein the scaffolds comprise adenosine. In an aspect of a disclosed method, a disclosed biomaterial can comprise a poly(ethylene glycol)-co-6 aminocaproic acid hydrogel. In an aspect of a disclosed method, a disclosed biomaterial can comprise a poly(ethylene glycol)-co-6 aminocaproic acid hydrogel, wherein the hydrogel comprises macroporous scaffolds. In an aspect of a disclosed method, a disclosed biomaterial can comprise a poly(ethylene glycol)-co-6 aminocaproic acid hydrogel, wherein the hydrogel comprises macroporous scaffolds comprising 3-acrylamido phenylboronic acid (3-APBA). In an aspect of a disclosed method, a disclosed biomaterial can comprise comprising a poly(ethylene glycol)-co-6 aminocaproic acid hydrogel comprising adenosine. In an aspect of a disclosed method, a disclosed biomaterial can comprise a poly(ethylene glycol)-co-6 aminocaproic acid hydrogel, wherein the hydrogel comprises 3-acrylamido phenylboronic acid (3-APBA) and adenosine. In an aspect of a disclosed method, a disclosed biomaterial can comprise a poly(ethylene glycol)-co-6 aminocaproic acid hydrogel with conjugated 3-APBA, wherein the hydrogel comprises macroporous scaffolds and adenosine. In an aspect of a disclosed method, a disclosed biomaterial can comprise a macroporous hydrogel, wherein the macroporous hydrogel are functionalized with 3-acrylamido phenylboronic acid (3-APBA). In an aspect of a disclosed method, a disclosed biomaterial can comprise a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine. In an aspect of a disclosed method, a disclosed poly(ethylene glycol) diacrylate (PEGDA) can be derived from or generated using poly(ethylene glycol) (PEG). In an aspect of a disclosed method, a disclosed N-acryloyl-6-aminocaproic acid (A6ACA) can be derived from or generated using 6-aminocaproic acid (6ACA). In an aspect of a disclosed method, a disclosed poly(ethylene glycol)-co-6 aminocaproic acid macroporous hydrogel can be derived from or generated using poly(ethylene glycol) (PEG) and using 6-aminocaproic acid (6ACA).

In an aspect of a disclosed method, a disclosed biomaterial can comprise a therapeutically effective amount of adenosine for treating, preventing, and/or mitigating pain.

In an aspect of a disclosed method, a disclosed therapeutically effective amount of adenosine can target adenosine receptor subtypes in one or more cell populations. For example, in an aspect, one or more disclosed cell populations can comprise bone marrow progenitor cells, mesenchymal stromal cells, immune cells, myeloid cells, inflammatory cells, LepR (+) lineage cells, dorsal root ganglion cells, peripheral nervous system neurons, central nervous system neurons, or any combination thereof.

In an aspect, a disclosed biomaterial can comprise from about 0.10 mg to about 1.0 mg adenosine per 1 mg of the disclosed biomaterial. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed biomaterial. In an aspect, a disclosed biomaterial can comprise more than 1.0 mg adenosine per 1 mg of the disclosed biomaterial.

In an aspect, a disclosed biomaterial can comprise from about 0.10 mg to about 1.0 mg adenosine per 1 mg of the disclosed macroporous hydrogel. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous hydrogel. In an aspect, a disclosed biomaterial can comprise more than 1.0 mg adenosine per 1 mg of the disclosed macroporous hydrogel.

In an aspect, a disclosed biomaterial can comprise from about 0.10 mg to about 1.0 mg adenosine per 1 mg of the disclosed scaffolds. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed scaffolds. In an aspect, a disclosed biomaterial can comprise more than 1.0 mg adenosine per 1 mg of the disclosed scaffolds.

In an aspect, for example, a disclosed biomaterial can comprise from about 1 mg/kg to about 30 mg/kg of ADO to body weight. In an aspect, for example, a disclosed biomaterial can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed biomaterial can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect of a disclosed method of treating, preventing, and/or mitigating pain, a disclosed subject can be diagnosed with or can be suspected of having chronic pain, acute pain, post-surgical pain, cancer pain, injury pain, pain associated with bone fracture, or any a combination thereof. In an aspect, a disclosed subject can have pain due to a non-union of one or more fractures.

In an aspect, a disclosed subject can have non-fracture pain. In an aspect, a disclosed subject can have neuropathic pain. In an aspect, a disclosed subject can have pain due to a burn injury. In an aspect, a disclosed subject can have pain due to one or more diagnosed conditions. In an aspect, a disclosed subject can have pain due an amputation (e.g., phantom pain).

In an aspect of a disclosed method of treating, preventing, and/or mitigating pain, a disclosed subject can be diagnosed with or can be suspected of having a bone fracture or more than one bone fractures. In an aspect, a disclosed subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a disclosed fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a disclosed subject can have one or more types of fractures. In an aspect, a disclosed method can comprise identifying the bone or bones having a fracture or fractures.

In an aspect of disclosed method of treating, preventing, and/or mitigating pain, administering the biomaterial can comprise implanting the biomaterial at the site of the subject's bone fracture, at the site of one or more of the subject's bone fractures, or at the site of every one of the subject's bone fractures. In an aspect in a disclosed method of treating, preventing, and/or mitigating pain, the bone fracture can be the result of an injury, a surgery, or a physical trauma. In an aspect, bone fracture can be the result of a non-trauma, a disease, or a disorder. In an aspect, a disclosed method can comprise implanting an orthopedic implant at the site of the subject's bone fracture, at the site of one or more of the subject's bone fractures, or at the site of every one of the subject's bone fractures. In an aspect, a disclosed orthopedic implant can be coated with the biomaterial.

In an aspect of disclosed method of treating, preventing, and/or mitigating pain, administering the biomaterial can comprise implanting a disclosed biomaterial at the site of the subject's pain. In an aspect of disclosed method of treating, preventing, and/or mitigating pain, administering the biomaterial can comprise locally implanting a disclosed biomaterial. In an aspect, a subject can have acute pain or chronic pain, post-surgical pain, cancer pain, injury pain, and/or pain associated with bone fracture.

In an aspect of a disclosed method, administering the biomaterial can comprise systemic administration. Systemic administration can comprise continuous administration, which can for example, comprise the use of an infusion pump. System administration can comprise non-continuous or intermittent administration.

In an aspect, a subject can be a non-human mammal or a human. A subject can be of any age, such as, for example, a geriatric, an adult, an adolescent, a child, or a baby. In an aspect, a subject can be diagnosed with or can be suspected of having a bone fracture, a local infection, a malabsorption syndrome, a sex hormone deficiency (hypogonadism), a systemic infection, AIDS/HIV, cancer, chronic kidney disease, chronic liver disease, chronic obstructive pulmonary disease, Cushing's syndrome, diabetes, gout, leukemia, low bone density, low bone density, lymphoma, metastatic cancer, multiple myeloma, one or more bone fractures, osteogenesis imperfecta, osteomalacia, osteonecrosis, osteoporosis, other rheumatological conditions, Paget's disease, pituitary disease, primary hyperparathyroidism, rheumatoid arthritis, Rickets, Scoliosis, stress fractures, Thalassemia major, untreated hyperthyroidism, or a combination thereof. In an aspect, a subject can have opioid dependence or can have recovered from an opioid dependence. In an aspect, a subject can be in active recovery from an opioid dependence.

In an aspect of a disclosed method, following the administering step, the subject experiences less pain, less intense pain, a shorter duration of pain, or a combination thereof.

In an aspect, a disclosed method of treating, preventing, and/or mitigating pain can comprise administering of one or more therapeutic agents. In an aspect, disclosed therapeutic agents can comprise biologically active agents, pharmaceutically active agents, anti-bacterial agents, anti-fungal agents, anti-viral agents, corticosteroids, analgesics, immunostimulants, immune-based products, blood-derived products, or a combination thereof. In an aspect, a disclosed therapeutic agent can comprise an opioid. In an aspect, the one or more therapeutic agents can be co-formulated with a disclosed biomaterial. In an aspect, the one or more therapeutic agents can be not co-formulated with a disclosed biomaterial. Therapeutic agents are known to the art and are disclosed herein.

In an aspect of a disclosed method of treating, preventing, and/or mitigating pain, administering the one or more disclosed therapeutic agents can comprise systemic administration, which can be, for example, continuous administration, non-continuous administration, or intermittent administration. In an aspect, continuous administration can comprise the use of an infusion pump.

In an aspect of a disclosed method of treating, preventing, and/or mitigating pain, the one or more therapeutic agents can be administered prior to, concurrently with, or after the administration of the disclosed biomaterial.

In an aspect, a disclosed method can comprise modifying one or more administering steps. In an aspect, modifying can comprise changing the amount of a disclosed biomaterial administered to the subject, the amount of the one or more disclosed therapeutic agents administered to the subject, or any combination thereof. In an aspect, changing the amount of the disclosed biomaterial or the one or more disclosed therapeutic agents administered to the subject can comprise increasing or decreasing the amount administered in the administering step. In an aspect, modifying the administering step can comprise changing the frequency of administration of the disclosed biomaterial or the one or more disclosed therapeutic agents to the subject. In an aspect, changing the frequency of administration can comprise increasing or decreasing the frequency of the administering step. In an aspect, modifying the administering can comprise changing the duration of administration of the disclosed biomaterial or the one or more therapeutic agents to the subject. In an aspect, changing the duration of administration can comprise increasing or decreasing the duration of the administering step. In an aspect, modifying can comprise modifying any aspect of a disclosed method.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect, a disclosed method can comprise reducing the subject's pain. In an aspect, a disclosed method can comprise assessing the subject's pain. The subject's pain can be assessed subjectively or objectively as known to the art.

In an aspect, a disclosed method can comprise improving the subject's ability to move. Any improvement in the subject's ability to move can be assessed subjectively or objectively as known to the art.

In an aspect, a disclosed method can comprise repeating the administering of and/or the implanting of the biomaterial, repeating the administering of the one or more therapeutic agents, repeating the administering of and/or implanting of the biomaterial and the one or more therapeutic agents, or any combination of administering steps thereof.

In an aspect, a disclosed method of treating, preventing, and/or mitigating pain can comprise promoting bone fracture healing in the subject, repairing or partially repairing a skeletal defect in the subject, or any combination thereof.

In an aspect, a disclosed method of treating, preventing, and/or mitigating pain can comprise enhancing the innate ability of bone repair tissue to repair bone in the subject, enhancing the outcome of orthopedic implant surgery in the subject, or a combination thereof. In an aspect, a disclosed method can comprise reducing bone degeneration in the subject, promoting bone regeneration in the subject, or a combination thereof. In an aspect, a disclosed method can comprise comprising promoting osteoblastogenesis in and around the area of the bone fracture, decreasing osteoclastogenesis in and around the area of the bone fracture, or a combination thereof.

In an aspect, a disclosed method can comprise promoting healing.

In an aspect, a disclosed method can promote osteogenic differentiation of mesenchymal stromal cells (MSCs). In an aspect of a disclosed method, promoting osteogenic differential of MSCs can comprise activation of ADORA2B and/or activity at ADORA2B.

In an aspect of a disclosed method, promoting osteogenic differentiation of MSCs can comprise activation of ADORA2A and/or activity at ADORA2A. In an aspect of a disclosed method, promoting osteogenic differentiation of MSCs can comprise activation of ADORA2A and/or ADORA2B and/or activity at ADORA2A and/or ADORA2B.

In an aspect, a disclosed method can comprise attenuating A1R or ADORA1 activation in the subject. In an aspect, a disclosed method of treating, preventing, and/or mitigating pain in a subject can comprise attenuating A3R or ADORA3 activation in the subject. In an aspect, a disclosed method can comprise decreasing activation of one or more dorsal root ganglions (DRGs).

In an aspect, a disclosed method of treating, preventing, and/or mitigating pain in a subject can comprise improving the overall functional outcome for the subject. Overall functional outcome can be measured objectively and/or subjectively by the skilled person in the art and/or by the subject and can include functions such as resuming work activities, engaging in recreational activities, and participating in daily functions without pain and/or without restriction. Other functions are known to the subject and/or skilled person.

In an aspect, a disclosed method can treat, prevent, and/or mitigate pain in a subject and can improve and/or promote healing of the subject's injury (such as, for example, one or more bone fractures and/or one or more soft tissue injuries).

In an aspect, a disclosed method can prevent a subject's pain from becoming chronic pain. In an aspect, a disclosed method can prevent a subject's pain from becoming maladaptive pain.

In an aspect, a disclosed method can attenuate NGF-induced sensitization of DRG neurons. In an aspect, attenuation can comprise a decrease of activity of ADORA1 and/or a decrease of expression of ADORA1.

In an aspect, a disclosed method of treating, preventing, and/or mitigating pain in a subject can decrease pain in the subject. In an aspect, an improvement can comprise about a 10% decrease, about a 20% decrease, about a 30% decrease, about a 40% decrease, about at 50% decrease, about at 60% decrease, about a 70% decrease, about a 80% decrease, about a 90% decrease, or about a 100% decrease in pain as compared to a pre-administration or a pre-implantation level and/or degree of pain.

In an aspect, a disclosed method of treating, preventing, and/or mitigating pain in a subject can improve weight bearing in the subject. In an aspect, an improvement can comprise about a 10% increase, about a 20% increase, about a 30% increase, about a 40% increase, about at 50% increase, about at 60% increase, about a 70% increase, about a 80% increase, about a 90% increase, or about a 100% increase in weight bearing as compared to a pre-administration or a pre-implantation level and/or degree of weight bearing.

In an aspect, a disclosed method of treating, preventing, and/or mitigating pain in a subject can improve movement in the subject. In an aspect, an improvement can comprise about a 10% increase, about a 20% increase, about a 30% increase, about a 40% increase, about at 50% increase, about at 60% increase, about a 70% increase, about a 80% increase, about a 90% increase, or about a 100% increase in movement as compared to a pre-administration or a pre-implantation level and/or degree of movement.

In an aspect, a disclosed method of treating, preventing, and/or mitigating pain in a subject can provide an increase in non-opioid analgesia in the subject. In an aspect, an increase in non-opioid analgesia can comprise about a 10% increase, about a 20% increase, about a 30% increase, about a 40% increase, about at 50% increase, about at 60% increase, about a 70% increase, about a 80% increase, about a 90% increase, or about a 100% increase in analgesia as compared to a pre-administration or a pre-implantation level and/or degree of analgesia.

### 2. Methods of Providing Non-Opioid Analgesia

Disclosed herein is a method of providing non-opioid analgesia, the method comprising administering to a subject a biomaterial comprising a therapeutically effective amount of adenosine. Disclosed herein is a method of providing non-opioid analgesia, the method comprising administering to a subject a biomaterial comprising a therapeutically effective amount of adenosine; wherein the subject's pain is reduced. Disclosed herein is a method of providing non-opioid analgesia, the method comprising administering to a subject a biomaterial comprising a therapeutically effective amount of adenosine; thereby reducing the subject's pain. Disclosed herein is a method of providing non-opioid analgesia, the method comprising implanting in a subject in need thereof a biomaterial comprising a therapeutically effective amount of adenosine.

Disclosed herein is a method of providing non-opioid analgesia, the method comprising implanting in a subject in need thereof a biomaterial comprising a therapeutically effective amount of adenosine; wherein the subject's pain is reduced. Disclosed herein is a method of providing non-opioid analgesia, the method comprising implanting in a subject in need thereof a biomaterial comprising a therapeutically effective amount of adenosine; thereby reducing the subject's pain. Disclosed herein is a method, the method comprising providing non-opioid analgesia by administering to a subject in need thereof a biomaterial comprising a therapeutically effective amount of adenosine; thereby reducing the subject's pain. Disclosed herein is a method, the method comprising providing non-opioid analgesia by implanting in a subject in need thereof a biomaterial comprising a therapeutically effective amount of adenosine; thereby reducing the subject's pain.

In an aspect of a disclosed method, a disclosed biomaterial can comprise a macroporous hydrogel, macroporous scaffolds, or a hydrogel comprising macroporous scaffolds. In an aspect of a disclosed method, a disclosed biomaterial can comprise a hydrogel or macroporous scaffolds. In an aspect of a disclosed method, macroporous scaffolds can comprise 3-APBA.

In an aspect of a disclosed method, a disclosed biomaterial can comprise poly(ethylene glycol)-co-6 aminocaproic acid macroporous scaffolds. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA). In an aspect of a disclosed method, a disclosed biomaterial can comprise macroporous scaffolds, wherein the scaffolds comprise a poly(ethylene glycol)-co-6 aminocaproic acid hydrogel comprising 3-acrylamido phenylboronic acid (3-APBA). In an aspect of a disclosed method, a disclosed biomaterial can comprise poly(ethylene glycol)-co-6 aminocaproic acid macroporous scaffolds comprising adenosine. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA) and adenosine. In an aspect of a disclosed method, a disclosed biomaterial can comprise macroporous scaffolds, wherein the scaffolds comprise a poly(ethylene glycol)-co-6 aminocaproic acid hydrogel comprises 3-acrylamido phenylboronic acid (3-APBA) and adenosine. In an aspect of a disclosed method, a disclosed biomaterial can comprise poly(ethylene glycol)-co-6 aminocaproic acid macroporous scaffolds, wherein the scaffolds comprise adenosine. In an aspect of a disclosed method, a disclosed biomaterial can comprise comprising 3-APBA-conjugated poly(ethylene glycol)-co-6 aminocaproic acid macroporous scaffolds, wherein the scaffolds comprise adenosine. In an aspect of a disclosed method, a disclosed biomaterial can comprise a poly(ethylene glycol)-co-6 aminocaproic acid hydrogel. In an aspect of a disclosed method, a disclosed biomaterial can comprise a poly(ethylene glycol)-co-6 aminocaproic acid hydrogel, wherein the hydrogel comprises macroporous scaffolds. In an aspect of a disclosed method, a disclosed biomaterial can comprise a poly(ethylene glycol)-co-6 aminocaproic acid hydrogel, wherein the hydrogel comprises macroporous scaffolds comprising 3-acrylamido phenylboronic acid (3-APBA). In an aspect of a disclosed method, a disclosed biomaterial can comprise comprising a poly(ethylene glycol)-co-6 aminocaproic acid hydrogel comprising adenosine. In an aspect of a disclosed method, a disclosed biomaterial can comprise a poly(ethylene glycol)-co-6 aminocaproic acid hydrogel, wherein the hydrogel comprises 3-acrylamido phenylboronic acid (3-APBA) and adenosine. In an aspect of a disclosed method, a disclosed biomaterial can comprise a poly(ethylene glycol)-co-6 aminocaproic acid hydrogel with conjugated 3-APBA, wherein the hydrogel comprises macroporous scaffolds and adenosine. In an aspect of a disclosed method, a disclosed biomaterial can comprise a macroporous hydrogel, wherein the macroporous hydrogel are functionalized with 3-acrylamido phenylboronic acid (3-APBA). In an aspect of a disclosed method, a disclosed biomaterial can comprise a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine. In an aspect of a disclosed method, a disclosed poly(ethylene glycol) diacrylate (PEGDA) can be derived from or generated using poly(ethylene glycol) (PEG). In an aspect of a disclosed method, a disclosed N-acryloyl-6-aminocaproic acid (A6ACA) can be derived from or generated using 6-aminocaproic acid (6ACA). In an aspect of a disclosed method, a disclosed poly(ethylene glycol)-co-6 aminocaproic acid macroporous hydrogel can be derived from or generated using poly(ethylene glycol) (PEG) and using 6-aminocaproic acid (6ACA).

In an aspect of a disclosed method of providing non-opioid analgesia, a disclosed biomaterial can comprise a therapeutically effective amount of adenosine for treating, preventing, and/or mitigating pain. In an aspect, a disclosed method of providing non-opioid analgesia can comprise administering a therapeutically effective amount of adenosine. In an aspect of a disclosed method of providing non-opioid analgesia, a disclosed therapeutically effective amount of adenosine can target adenosine receptor subtypes in one or more cell populations. For example, in an aspect, one or more disclosed cell populations can comprise bone marrow progenitor cells, mesenchymal stromal cells, immune cells, myeloid cells, inflammatory cells, LepR (+) lineage cells, dorsal root ganglion cells, peripheral nervous system neurons, central nervous system neurons, or any combination thereof.

In an aspect of a disclosed method of providing non-opioid analgesia, a disclosed biomaterial can comprise from about 0.10 mg to about 1.0 mg adenosine per 1 mg of the disclosed biomaterial. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed biomaterial. In an aspect, a disclosed biomaterial can comprise more than 1.0 mg adenosine per 1 mg of the disclosed biomaterial.

In an aspect of a disclosed method of providing non-opioid analgesia, a disclosed biomaterial can comprise from about 0.10 mg to about 1.0 mg adenosine per 1 mg of the disclosed macroporous hydrogel. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous hydrogel. In an aspect, a disclosed biomaterial can comprise more than 1.0 mg adenosine per 1 mg of the disclosed macroporous hydrogel.

In an aspect of a disclosed method of providing non-opioid analgesia, a disclosed biomaterial can comprise from about 0.10 mg to about 1.0 mg adenosine per 1 mg of the disclosed scaffolds. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed scaffolds. In an aspect, a disclosed biomaterial can comprise more than 1.0 mg adenosine per 1 mg of the disclosed scaffolds.

In an aspect, for example, a disclosed biomaterial can comprise from about 1 mg/kg to about 30 mg/kg of ADO to body weight. In an aspect, for example, a disclosed biomaterial can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed biomaterial can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect of a disclosed method of providing non-opioid analgesia, a disclosed subject can be diagnosed with or can be suspected of having chronic pain, acute pain, post-surgical pain, cancer pain, injury pain, pain associated with bone fracture, or any a combination thereof. In an aspect, a disclosed subject can have pain due to a non-union of one or more fractures.

In an aspect of a disclosed method of providing non-opioid analgesia, a disclosed subject can have non-fracture pain. In an aspect, a disclosed subject can have neuropathic pain. In an aspect, a disclosed subject can have pain due to a burn injury. In an aspect, a disclosed subject can have pain due to one or more diagnosed conditions. In an aspect, a disclosed subject can have pain due an amputation (e.g., phantom pain).

In an aspect of a disclosed method of providing non-opioid analgesia, a disclosed subject can be diagnosed with or can be suspected of having a bone fracture or more than one bone fractures. In an aspect, a disclosed subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a disclosed fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a disclosed subject can have one or more types of fractures. In an aspect, a disclosed method can comprise identifying the bone or bones having a fracture or fractures.

In an aspect of a disclosed method of providing non-opioid analgesia, administering the biomaterial can comprise implanting the biomaterial at the site of the subject's bone fracture, at the site of one or more of the subject's bone fractures, or at the site of every one of the subject's bone fractures. In an aspect of a disclosed method of providing non-opioid analgesia, the bone fracture can be the result of an injury, a surgery, or a physical trauma. In an aspect, bone fracture can be the result of a non-trauma, a disease, or a disorder. In an aspect, a disclosed method can comprise implanting an orthopedic implant at the site of the subject's bone fracture, at the site of one or more of the subject's bone fractures, or at the site of every one of the subject's bone fractures. In an aspect, a disclosed orthopedic implant can be coated with the biomaterial.

In an aspect of a disclosed method of providing non-opioid analgesia, administering the biomaterial can comprise implanting a disclosed biomaterial at the site of the subject's pain. In an aspect of disclosed method of providing non-opioid analgesia, administering the biomaterial can comprise locally implanting a disclosed biomaterial. In an aspect, a subject can have acute pain or chronic pain, post-surgical pain, cancer pain, injury pain, and/or pain associated with bone fracture.

In an aspect of a disclosed method of providing non-opioid analgesia, administering the biomaterial can comprise systemic administration. Systemic administration can comprise continuous administration, which can for example, comprise the use of an infusion pump. System administration can comprise non-continuous or intermittent administration.

In an aspect, a subject can be a non-human mammal or a human. A subject can be of any age, such as, for example, a geriatric, an adult, an adolescent, a child, or a baby. In an aspect, a subject can be diagnosed with or can be suspected of having a bone fracture, a local infection, a malabsorption syndrome, a sex hormone deficiency (hypogonadism), a systemic infection, AIDS/HIV, cancer, chronic kidney disease, chronic liver disease, chronic obstructive pulmonary disease, Cushing's syndrome, diabetes, gout, leukemia, low bone density, low bone density, lymphoma, metastatic cancer, multiple myeloma, one or more bone fractures, osteogenesis imperfecta, osteomalacia, osteonecrosis, osteoporosis, other rheumatological conditions, Paget's disease, pituitary disease, primary hyperparathyroidism, rheumatoid arthritis, Rickets, Scoliosis, stress fractures, Thalassemia major, untreated hyperthyroidism, or a combination thereof. In an aspect, a subject can have opioid dependence or can have recovered from an opioid dependence. In an aspect, a subject can be in active recovery from an opioid dependence.

In an aspect of a disclosed method of providing non-opioid analgesia, following the administering step and/or the implanting step, the subject experiences less pain, less intense pain, a shorter duration of pain, or a combination thereof.

In an aspect, a disclosed method of providing non-opioid analgesia can comprise administering of one or more therapeutic agents. In an aspect, disclosed therapeutic agents can comprise biologically active agents, pharmaceutically active agents, anti-bacterial agents, anti-fungal agents, anti-viral agents, corticosteroids, analgesics, immunostimulants, immune-based products, blood-derived products, or a combination thereof. In an aspect, a disclosed therapeutic agent can comprise an opioid. In an aspect, the one or more therapeutic agents can be co-formulated with a disclosed biomaterial. In an aspect, the one or more therapeutic agents can be not co-formulated with a disclosed biomaterial. Therapeutic agents are known to the art and are disclosed herein.

In an aspect of a disclosed method of providing non-opioid analgesia, administering the one or more disclosed therapeutic agents can comprise systemic administration, which can be, for example, continuous administration, non-continuous administration, or intermittent administration. In an aspect, continuous administration can comprise the use of an infusion pump.

In an aspect of a disclosed method of providing non-opioid analgesia, the one or more therapeutic agents can be administered prior to, concurrently with, or after the administration of the disclosed biomaterial.

In an aspect, a disclosed method of providing non-opioid analgesia can comprise modifying one or more administering steps. In an aspect, modifying can comprise changing the amount of a disclosed biomaterial administered to the subject, the amount of the one or more disclosed therapeutic agents administered to the subject, or any combination thereof. In an aspect, changing the amount of the disclosed biomaterial or the one or more disclosed therapeutic agents administered to the subject can comprise increasing or decreasing the amount administered in the administering step. In an aspect, modifying the administering step can comprise changing the frequency of administration of the disclosed biomaterial or the one or more disclosed therapeutic agents to the subject. In an aspect, changing the frequency of administration can comprise increasing or decreasing the frequency of the administering step. In an aspect, modifying the administering can comprise changing the duration of administration of the disclosed biomaterial or the one or more therapeutic agents to the subject. In an aspect, changing the duration of administration can comprise increasing or decreasing the duration of the administering step. In an aspect, modifying can comprise modifying any aspect of a disclosed method.

In an aspect, a disclosed method of providing non-opioid analgesia can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect, a disclosed method of providing non-opioid analgesia can comprise reducing the subject's pain. In an aspect, a disclosed method can comprise assessing the subject's pain. The subject's pain can be assessed subjectively or objectively as known to the art.

In an aspect, a disclosed method of providing non-opioid analgesia can comprise improving the subject's ability to move. Any improvement in the subject's ability to move can be assessed subjectively or objectively as known to the art.

In an aspect, a disclosed method of providing non-opioid analgesia can comprise repeating the administering of and/or implanting of the biomaterial, repeating the administering of the one or more therapeutic agents, repeating the administering of and/or the implanting of the biomaterial and the one or more therapeutic agents, or any combination of administering steps thereof.

In an aspect, a disclosed method of providing non-opioid analgesia can comprise promoting bone fracture healing in the subject, repairing or partially repairing a skeletal defect in the subject, or any combination thereof.

In an aspect, a disclosed method of providing non-opioid analgesia can comprise enhancing the innate ability of bone repair tissue to repair bone in the subject, enhancing the outcome of orthopedic implant surgery in the subject, or a combination thereof. In an aspect, a disclosed method can comprise reducing bone degeneration in the subject, promoting bone regeneration in the subject, or a combination thereof. In an aspect, a disclosed method can comprise comprising promoting osteoblastogenesis in and around the area of the bone fracture, decreasing osteoclastogenesis in and around the area of the bone fracture, or a combination thereof.

In an aspect, a disclosed method of providing non-opioid analgesia can comprise promoting healing.

In an aspect, a disclosed method of providing non-opioid analgesia can promote osteogenic differentiation of mesenchymal stromal cells (MSCs). In an aspect of a disclosed method, promoting osteogenic differential of MSCs can comprise activation of ADORA2B and/or activity at ADORA2B. In an aspect of a disclosed method of providing non-opioid analgesia, promoting osteogenic differentiation of MSCs can comprise activation of ADORA2A and/or activity at ADORA2A. In an aspect of a disclosed method, promoting osteogenic differentiation of MSCs can comprise activation of ADORA2A and/or ADORA2B and/or activity at ADORA2A and/or ADORA2B. In an aspect, a disclosed method of providing non-opioid analgesia can comprise attenuating A1R or ADORA1 activation in the subject. In an aspect, a disclosed method of providing non-opioid analgesia can comprise attenuating A3R or ADORA3 activation in the subject. In an aspect, a disclosed method can comprise decreasing activation of one or more dorsal root ganglions (DRGs).

In an aspect, a disclosed method of providing non-opioid analgesia can comprise improving the overall functional outcome for the subject. Overall functional outcome can be measured objectively and/or subjectively by the skilled person in the art and/or by the subject and can include functions such as resuming work activities, engaging in recreational activities, and participating in daily functions without pain and/or without restriction. Other functions are known to the subject and/or skilled person.

In an aspect, a disclosed method of providing non-opioid analgesia can treat, prevent, and/or mitigate pain in a subject and can improve and/or promote healing of the subject's injury (such as, for example, one or more bone fractures and/or one or more soft tissue injuries).

In an aspect, a disclosed method of providing non-opioid analgesia can prevent a subject's pain from becoming chronic pain. In an aspect, a disclosed method can prevent a subject's pain from becoming maladaptive pain.

In an aspect, a disclosed method of providing non-opioid analgesia can attenuate NGF-induced sensitization of DRG neurons. In an aspect, attenuation can comprise a decrease of activity of ADORA1 and/or a decrease of expression of ADORA1.

In an aspect, a disclosed method of providing non-opioid analgesia can improve weight bearing in the subject. In an aspect, an improvement can comprise about a 10% increase, about a 20% increase, about a 30% increase, about a 40% increase, about at 50% increase, about at 60% increase, about a 70% increase, about am 80% increase, about a 90% increase, or about a 100% increase in weight bearing as compared to a pre-administration level and/or degree of weight bearing.

In an aspect, a disclosed method of providing non-opioid analgesia can decrease pain in the subject. In an aspect, an improvement can comprise about a 10% decrease, about a 20% decrease, about a 30% decrease, about a 40% decrease, about at 50% decrease, about at 60% decrease, about a 70% decrease, about an 80% decrease, about a 90% decrease, or about a 100% decrease in pain as compared to a pre-administration or a pre-implantation level and/or degree of pain.

In an aspect, a disclosed method of providing non-opioid analgesia can improve weight bearing in the subject. In an aspect, an improvement can comprise about a 10% increase, about a 20% increase, about a 30% increase, about a 40% increase, about at 50% increase, about at 60% increase, about a 70% increase, about a 80% increase, about a 90% increase, or about a 100% increase in weight bearing as compared to a pre-administration or a pre-implantation level and/or degree of weight bearing.

In an aspect, a disclosed method of providing non-opioid analgesia can improve movement in the subject. In an aspect, an improvement can comprise about a 10% increase, about a 20% increase, about a 30% increase, about a 40% increase, about at 50% increase, about at 60% increase, about a 70% increase, about a 80% increase, about a 90% increase, or about a 100% increase in movement as compared to a pre-administration or a pre-implantation level and/or degree of movement.

In an aspect, a disclosed method of providing non-opioid analgesia can provide an increase in non-opioid analgesia in the subject. In an aspect, an increase in non-opioid analgesia can comprise about a 10% increase, about a 20% increase, about a 30% increase, about a 40% increase, about at 50% increase, about at 60% increase, about a 70% increase, about a 80% increase, about a 90% increase, or about a 100% increase in analgesia as compared to a pre-administration or a pre-implantation level and/or degree of analgesia.

### 3. Methods of Improving Movement of a Subject

Disclosed herein is a method of improving movement of a subject, the method comprising administering to a subject having pain a biomaterial comprising a therapeutically effective amount of adenosine, wherein the subject's pain associated is reduced, thereby allowing the subject to improve movement. Disclosed herein is a method of improving movement of a subject, the method comprising administering to a subject having acute pain or chronic pain, post-surgical pain, cancer pain, injury pain, and/or pain associated with bone fracture a disclosed biomaterial, and reducing the subject's pain, thereby allowing the subject to improve movement. Disclosed herein is a method of improving movement in a subject, the method comprising improving a subject's movement by administering to the subject having pain a disclosed biomaterial, wherein the subject's pain is reduced.

Disclosed herein is a method of improving movement in a subject, the method comprising reducing a subject's pain by administering to the subject having a pain a disclosed biomaterial, thereby improving movement in the subject. Disclosed herein is a method of improving movement in a subject, the method comprising administering to a subject a disclosed biomaterial, wherein the subject's pain is reduced, thereby allowing the subject to improve movement. Disclosed herein is a method of improving movement in a subject, the method comprising administering to a subject having pain a biomaterial comprising a therapeutically effective amount of adenosine, and reducing the subject's pain, thereby allowing the subject to improve movement.

Disclosed herein is a method of improving movement in a subject, the method comprising administering to a subject having acute pain or chronic pain, post-surgical pain, cancer pain, injury pain, and/or pain associated with bone fracture a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject having pain a biomaterial comprising a therapeutically effective amount of adenosine, wherein the subject's pain is reduced. Disclosed herein is a method of improving movement in a subject, the method comprising reducing a subject's pain by administering to the subject having a pain a biomaterial comprising a therapeutically effective amount of adenosine, thereby improving movement in the subject.

Disclosed herein is a method of improving movement in a subject, the method comprising administering to a subject a biomaterial comprising a therapeutically effective amount of adenosine, wherein the subject's pain is reduced, thereby allowing the subject to improve movement. Disclosed herein is a method of improving movement in a subject, the method comprising administering to a subject having one or more bone fractures a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject having one or more bone fractures a biomaterial comprising a therapeutically effective amount of adenosine, wherein the subject's pain is reduced. Disclosed herein is a method of improving movement in a subject, the method comprising reducing a subject's by administering to the subject having one or more bone fractures a biomaterial comprising a therapeutically effective amount of adenosine, thereby improving movement in the subject.

Disclosed herein is a method of improving movement in a subject, the method comprising administering to a subject a biomaterial comprising a therapeutically effective amount of adenosine, wherein the subject's pain is reduced, thereby allowing the subject to improve movement. Disclosed herein is a method of improving movement in a subject, the method comprising improving a subject's movement by administering to the subject a biomaterial comprising a therapeutically effective amount of adenosine, wherein the subject's pain is reduced. Disclosed herein is a method of improving movement in a subject, the method comprising reducing a subject's by administering to the subject a biomaterial comprising a therapeutically effective amount of adenosine, thereby improving movement in the subject. Disclosed herein is a method, the method comprising improving a subject's movement by administering to a subject in need thereof a biomaterial comprising a therapeutically effective amount of adenosine; thereby reducing the subject's pain. Disclosed herein is a method, the method comprising improving a subject's movement by implanting in a subject in need thereof a biomaterial comprising a therapeutically effective amount of adenosine; thereby reducing the subject's pain.

In an aspect of a disclosed method of improving movement, a disclosed biomaterial can comprise a macroporous hydrogel, macroporous scaffolds, or a hydrogel comprising macroporous scaffolds. In an aspect of a disclosed method, a disclosed biomaterial can comprise a hydrogel or macroporous scaffolds. In an aspect of a disclosed method, macroporous scaffolds can comprise 3-APBA.

In an aspect of a disclosed method of improving movement, a disclosed biomaterial can comprise poly(ethylene glycol)-co-6 aminocaproic acid macroporous scaffolds. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA). In an aspect of a disclosed method, a disclosed biomaterial can comprise macroporous scaffolds, wherein the scaffolds comprise a poly(ethylene glycol)-co-6 aminocaproic acid hydrogel comprising 3-acrylamido phenylboronic acid (3-APBA). In an aspect of a disclosed method, a disclosed biomaterial can comprise poly(ethylene glycol)-co-6 aminocaproic acid macroporous scaffolds comprising adenosine. In an aspect of a disclosed method, a disclosed biomaterial can comprise poly(ethylene glycol)-co-6 aminocaproic acid macroporous scaffolds, wherein the scaffolds comprise 3-acrylamido phenylboronic acid (3-APBA) and adenosine. In an aspect of a disclosed method, a disclosed biomaterial can comprise macroporous scaffolds, wherein the scaffolds comprise a poly(ethylene glycol)-co-6 aminocaproic acid hydrogel comprises 3-acrylamido phenylboronic acid (3-APBA) and adenosine. In an aspect of a disclosed method, a disclosed biomaterial can comprise poly(ethylene glycol)-co-6 aminocaproic acid macroporous scaffolds, wherein the scaffolds comprise adenosine. In an aspect of a disclosed method, a disclosed biomaterial can comprise comprising 3-APBA-conjugated poly(ethylene glycol)-co-6 aminocaproic acid macroporous scaffolds, wherein the scaffolds comprise adenosine. In an aspect of a disclosed method, a disclosed biomaterial can comprise a poly(ethylene glycol)-co-6 aminocaproic acid hydrogel. In an aspect of a disclosed method, a disclosed biomaterial can comprise a poly(ethylene glycol)-co-6 aminocaproic acid hydrogel, wherein the hydrogel comprises macroporous scaffolds. In an aspect of a disclosed method, a disclosed biomaterial can comprise a poly(ethylene glycol)-co-6 aminocaproic acid hydrogel, wherein the hydrogel comprises macroporous scaffolds comprising 3-acrylamido phenylboronic acid (3-APBA). In an aspect of a disclosed method, a disclosed biomaterial can comprise comprising a poly(ethylene glycol)-co-6 aminocaproic acid hydrogel comprising adenosine. In an aspect of a disclosed method, a disclosed biomaterial can comprise a poly(ethylene glycol)-co-6 aminocaproic acid hydrogel, wherein the hydrogel comprises 3-acrylamido phenylboronic acid (3-APBA) and adenosine. In an aspect of a disclosed method, a disclosed biomaterial can comprise a poly(ethylene glycol)-co-6 aminocaproic acid hydrogel with conjugated 3-APBA, wherein the hydrogel comprises macroporous scaffolds and adenosine. In an aspect of a disclosed method, a disclosed biomaterial can comprise a macroporous hydrogel, wherein the macroporous hydrogel are functionalized with 3-acrylamido phenylboronic acid (3-APBA). In an aspect of a disclosed method, a disclosed biomaterial can comprise a poly(ethylene glycol)-co-6 aminocaproic acid macroporous hydrogel, wherein the macroporous hydrogel are functionalized with 3-acrylamido phenylboronic acid (3-APBA), wherein the macroporous hydrogel comprises adenosine. In an aspect of a disclosed method, a disclosed poly(ethylene glycol) diacrylate (PEGDA) can be derived from or generated using poly(ethylene glycol) (PEG). In an aspect of a disclosed method, a disclosed N-acryloyl-6-aminocaproic acid (A6ACA) can be derived from or generated using 6-aminocaproic acid (6ACA). In an aspect of a disclosed method, a disclosed poly(ethylene glycol)-co-6 aminocaproic acid macroporous hydrogel can be derived from or generated using poly(ethylene glycol) (PEG) and using 6-aminocaproic acid (6ACA).

In an aspect of a disclosed method of improving movement, a disclosed biomaterial can comprise a therapeutically effective amount of adenosine for treating, preventing, and/or mitigating pain.

In an aspect of a disclosed method of improving movement, a disclosed therapeutically effective amount of adenosine can target adenosine receptor subtypes in one or more cell populations. For example, in an aspect, one or more disclosed cell populations can comprise bone marrow progenitor cells, mesenchymal stromal cells, immune cells, myeloid cells, inflammatory cells, LepR (+) lineage cells, dorsal root ganglion cells, peripheral nervous system neurons, central nervous system neurons, or any combination thereof.

In an aspect, a disclosed biomaterial can comprise from about 0.10 mg to about 1.0 mg adenosine per 1 mg of the disclosed biomaterial. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed biomaterial. In an aspect, a disclosed biomaterial can comprise more than 1.0 mg adenosine per 1 mg of the disclosed biomaterial.

In an aspect, a disclosed biomaterial can comprise from about 0.10 mg to about 1.0 mg adenosine per 1 mg of the disclosed macroporous hydrogel. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed macroporous hydrogel. In an aspect, a disclosed biomaterial can comprise more than 1.0 mg adenosine per 1 mg of the disclosed macroporous hydrogel.

In an aspect, a disclosed biomaterial can comprise from about 0.10 mg to about 1.0 mg adenosine per 1 mg of the disclosed scaffolds. In an aspect, a disclosed biomaterial can comprise from about 0.10 mg, about 0.20 mg, about 0.30 mg, about 0.40 mg, about 0.50 mg, about 0.60 mg, about 0.70 mg, about 0.80 mg, about 0.90 mg, about 1.0 mg adenosine per 1 mg of the disclosed scaffolds. In an aspect, a disclosed biomaterial can comprise more than 1.0 mg adenosine per 1 mg of the disclosed scaffolds.

In an aspect, for example, a disclosed biomaterial can comprise from about 1 mg/kg to about 30 mg/kg of ADO to body weight. In an aspect, for example, a disclosed biomaterial can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed biomaterial can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

In an aspect of a disclosed method of improving movement, a disclosed subject can be diagnosed with or can be suspected of having chronic pain, acute pain, post-surgical pain, cancer pain, injury pain, pain associated with bone fracture, or any a combination thereof. In an aspect, a disclosed subject can have pain due to a non-union of one or more fractures.

In an aspect, a disclosed subject can have non-fracture pain. In an aspect, a disclosed subject can have neuropathic pain. In an aspect, a disclosed subject can have pain due to a burn injury. In an aspect, a disclosed subject can have pain due to one or more diagnosed conditions. In an aspect, a disclosed subject can have pain due an amputation (e.g., phantom pain).

In an aspect of a disclosed method of improving movement, a disclosed subject can be diagnosed with or can be suspected of having a bone fracture or more than one bone fractures. In an aspect, a disclosed subject can have multiple fractures in the same bone, multiple fractures in different bones, or both. In an aspect of a disclosed method, a fracture can comprise an open fracture, a closed fracture, a partial fracture, a complete fracture, a stable fracture, or a displaced fracture. In an aspect of a disclosed method, a disclosed fracture comprises a transverse fracture, a spiral fracture, a greenstick fracture, a stress fracture, a compression fracture, an oblique fracture, an impacted fracture, a segmental fracture, a comminuted fracture, or an avulsion fracture. In an aspect, a disclosed subject can have one or more types of fractures. In an aspect, a disclosed method can comprise identifying the bone or bones having a fracture or fractures.

In an aspect of disclosed method of improving movement, administering the biomaterial can comprise implanting the biomaterial at the site of the subject's bone fracture, at the site of one or more of the subject's bone fractures, or at the site of every one of the subject's bone fractures. In an aspect in a disclosed method of improving movement, the bone fracture can be the result of an injury, a surgery, or a physical trauma. In an aspect, bone fracture can be the result of a non-trauma, a disease, or a disorder. In an aspect, a disclosed method can comprise implanting an orthopedic implant at the site of the subject's bone fracture, at the site of one or more of the subject's bone fractures, or at the site of every one of the subject's bone fractures. In an aspect, a disclosed orthopedic implant can be coated with the biomaterial.

In an aspect of disclosed method of improving movement, administering the biomaterial can comprise implanting a disclosed biomaterial at the site of the subject's pain. In an aspect of disclosed method of improving movement, administering the biomaterial can comprise locally implanting a disclosed biomaterial. In an aspect, a subject can have acute pain or chronic pain, post-surgical pain, cancer pain, injury pain, and/or pain associated with bone fracture.

In an aspect of a disclosed method of improving movement, administering the biomaterial can comprise systemic administration. Systemic administration can comprise continuous administration, which can for example, comprise the use of an infusion pump. System administration can comprise non-continuous or intermittent administration.

In an aspect, a subject can be a non-human mammal or a human. A subject can be of any age, such as, for example, a geriatric, an adult, an adolescent, a child, or a baby. In an aspect, a subject can be diagnosed with or can be suspected of having a bone fracture, a local infection, a malabsorption syndrome, a sex hormone deficiency (hypogonadism), a systemic infection, AIDS/HIV, cancer, chronic kidney disease, chronic liver disease, chronic obstructive pulmonary disease, Cushing's syndrome, diabetes, gout, leukemia, low bone density, low bone density, lymphoma, metastatic cancer, multiple myeloma, one or more bone fractures, osteogenesis imperfecta, osteomalacia, osteonecrosis, osteoporosis, other rheumatological conditions, Paget's disease, pituitary disease, primary hyperparathyroidism, rheumatoid arthritis, Rickets, Scoliosis, stress fractures, Thalassemia major, untreated hyperthyroidism, or a combination thereof. In an aspect, a subject can have opioid dependence or can have recovered from an opioid dependence. In an aspect, a subject can be in active recovery from an opioid dependence.

In an aspect of a disclosed method of improving movement, following the administering and/or implanting step, the subject experiences less pain, less intense pain, a shorter duration of pain, or a combination thereof.

In an aspect, a disclosed method of improving movement can comprise administering of one or more therapeutic agents. In an aspect, disclosed therapeutic agents can comprise biologically active agents, pharmaceutically active agents, anti-bacterial agents, anti-fungal agents, anti-viral agents, corticosteroids, analgesics, immunostimulants, immune-based products, blood-derived products, or a combination thereof. In an aspect, a disclosed therapeutic agent can comprise an opioid. In an aspect, the one or more therapeutic agents can be co-formulated with a disclosed biomaterial. In an aspect, the one or more therapeutic agents can be not co-formulated with a disclosed biomaterial. Therapeutic agents are known to the art and are disclosed herein.

In an aspect of a disclosed method of improving movement, administering the one or more disclosed therapeutic agents can comprise systemic administration, which can be, for example, continuous administration, non-continuous administration, or intermittent administration. In an aspect, continuous administration can comprise the use of an infusion pump.

In an aspect of a disclosed method of improving movement, the one or more therapeutic agents can be administered prior to, concurrently with, or after the administration of the disclosed biomaterial.

In an aspect, a disclosed method of improving movement can comprise modifying one or more administering steps. In an aspect, modifying can comprise changing the amount of a disclosed biomaterial administered to the subject, the amount of the one or more disclosed therapeutic agents administered to the subject, or any combination thereof. In an aspect, changing the amount of the disclosed biomaterial or the one or more disclosed therapeutic agents administered to the subject can comprise increasing or decreasing the amount administered in the administering step. In an aspect, modifying the administering step can comprise changing the frequency of administration of the disclosed biomaterial or the one or more disclosed therapeutic agents to the subject. In an aspect, changing the frequency of administration can comprise increasing or decreasing the frequency of the administering step. In an aspect, modifying the administering can comprise changing the duration of administration of the disclosed biomaterial or the one or more therapeutic agents to the subject. In an aspect, changing the duration of administration can comprise increasing or decreasing the duration of the administering step. In an aspect, modifying can comprise modifying any aspect of a disclosed method.

In an aspect, a disclosed method of improving movement can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect, a disclosed method of improving movement can comprise reducing the subject's pain. In an aspect, a disclosed method can comprise assessing the subject's pain. The subject's pain can be assessed subjectively or objectively as known to the art.

In an aspect, a disclosed method can comprise improving the subject's ability to move. Any improvement in the subject's ability to move can be assessed subjectively or objectively as known to the art.

In an aspect, a disclosed method of improving movement can comprise repeating the administering of and/or implanting of the biomaterial, repeating the administering of the one or more therapeutic agents, repeating the administering of and/or implanting of the biomaterial and the one or more therapeutic agents, or any combination of administering steps thereof.

In an aspect, a disclosed method of improving movement can comprise promoting bone fracture healing in the subject, repairing or partially repairing a skeletal defect in the subject, or any combination thereof.

In an aspect, a disclosed method of improving movement can comprise enhancing the innate ability of bone repair tissue to repair bone in the subject, enhancing the outcome of orthopedic implant surgery in the subject, or a combination thereof. In an aspect, a disclosed method can comprise reducing bone degeneration in the subject, promoting bone regeneration in the subject, or a combination thereof. In an aspect, a disclosed method can comprise comprising promoting osteoblastogenesis in and around the area of the bone fracture, decreasing osteoclastogenesis in and around the area of the bone fracture, or a combination thereof.

In an aspect, a disclosed method can comprise promoting healing.

In an aspect, a disclosed method of improving movement can promote osteogenic differentiation of mesenchymal stromal cells (MSCs). In an aspect of a disclosed method, promoting osteogenic differential of MSCs can comprise activation of ADORA2B and/or activity at ADORA2B. In an aspect of a disclosed method of improving movement, promoting osteogenic differentiation of MSCs can comprise activation of ADORA2A and/or activity at ADORA2A. In an aspect of a disclosed method, promoting osteogenic differentiation of MSCs can comprise activation of ADORA2A and/or ADORA2B and/or activity at ADORA2A and/or ADORA2B. In an aspect, a disclosed method of improving movement can comprise attenuating A1R or ADORA1 activation in the subject. In an aspect, a disclosed method of treating, preventing, and/or mitigating pain in a subject can comprise attenuating A3R or ADORA3 activation in the subject. In an aspect, a disclosed method can comprise decreasing activation of one or more dorsal root ganglions (DRGs).

In an aspect, a disclosed method of improving movement in a subject can comprise improving the overall functional outcome for the subject. Overall functional outcome can be measured objectively and/or subjectively by the skilled person in the art and/or by the subject and can include functions such as resuming work activities, engaging in recreational activities, and participating in daily functions without pain and/or without restriction. Other functions are known to the subject and/or skilled person.

In an aspect, a disclosed method of improving movement can treat, prevent, and/or mitigate pain in a subject and can improve and/or promote healing of the subject's injury (such as, for example, one or more bone fractures and/or one or more soft tissue injuries). In an aspect, a disclosed method of improving movement can prevent a subject's pain from becoming chronic pain. In an aspect, a disclosed method can prevent a subject's pain from becoming maladaptive pain.

In an aspect, a disclosed method of improving movement can attenuate NGF-induced sensitization of DRG neurons. In an aspect, attenuation can comprise a decrease of activity of ADORA1 and/or a decrease of expression of ADORA1.

In an aspect, a disclosed method of improving movement can improve weight bearing in the subject. In an aspect, an improvement can comprise about a 10% increase, about a 20% increase, about a 30% increase, about a 40% increase, about at 50% increase, about at 60% increase, about a 70% increase, about an 80% increase, about a 90% increase, or about a 100% increase in weight bearing as compared to a pre-administration level and/or degree of weight bearing.

In an aspect, a disclosed method of improving movement can decrease pain in the subject. In an aspect, an improvement can comprise about a 10% decrease, about a 20% decrease, about a 30% decrease, about a 40% decrease, about at 50% decrease, about at 60% decrease, about a 70% decrease, about an 80% decrease, about a 90% decrease, or about a 100% decrease in pain as compared to a pre-administration or a pre-implantation level and/or degree of pain.

In an aspect, a disclosed method of improving movement can improve weight bearing in the subject. In an aspect, an improvement can comprise about a 10% increase, about a 20% increase, about a 30% increase, about a 40% increase, about at 50% increase, about at 60% increase, about a 70% increase, about an 80% increase, about a 90% increase, or about a 100% increase in weight bearing as compared to a pre-administration or a pre-implantation level and/or degree of weight bearing.

In an aspect, a disclosed method of improving movement can improve movement in the subject. In an aspect, an improvement can comprise about a 10% increase, about a 20% increase, about a 30% increase, about a 40% increase, about at 50% increase, about at 60% increase, about a 70% increase, about an 80% increase, about a 90% increase, or about a 100% increase in movement as compared to a pre-administration or a pre-implantation level and/or degree of movement.

In an aspect, a disclosed method of improving movement can provide an increase in non-opioid analgesia in the subject. In an aspect, an increase in non-opioid analgesia can comprise about a 10% increase, about a 20% increase, about a 30% increase, about a 40% increase, about at 50% increase, about at 60% increase, about a 70% increase, about an 80% increase, about a 90% increase, or about a 100% increase in analgesia as compared to a pre-administration or a pre-implantation level and/or degree of analgesia.

### 4. Methods of Making a Biomaterial

Disclosed herein is a method of making a disclosed biomaterial comprising adenosine. Disclosed herein is a method of making a disclosed biomaterial comprising a therapeutically effective amount of adenosine for treating, preventing, and/or mitigating pain.

Disclosed herein is a method of making a disclosed biomaterial comprising macroporous scaffolds. Disclosed herein is a method of making a disclosed biomaterial comprising a hydrogel. Disclosed herein is a method of making a disclosed biomaterial comprising macroporous scaffolds and adenosine. Disclosed herein is a method of making a disclosed biomaterial comprising a hydrogel and adenosine. Disclosed herein is a method of making a disclosed biomaterial comprising a hydrogel, wherein the hydrogel comprises macroporous scaffolds and adenosine.

Disclosed herein is a method of making a disclosed biomaterial, the method comprising: dissolving PEGDA, 3-APBA, A6ACA, and a radical photoinitiator into a mixture; adding the mixture to a mold packed with PMMA microbeads; UV irradiating the mold; removing the PMMA microbeads; and washing and rehydrating the porous hydrogel.

In an aspect, a disclosed mixture can comprise a 20:80 water:ethanol mixture.

In an aspect, PEGDA can comprise about 1% to about 20% w/v, or PEGDA can comprise about 10% w/v. In an aspect, 3-APBA can comprise about 0.1 M to about 2.0 M, or 3-APBA can comprise about 1.0 M. In an aspect, A6ACA can comprise about 0.1 M to about 2.0 M, or A6ACA can comprise about 0.5 M.

In an aspect, a radical photoinitiator can comprise a non-yellowing photoinitiator, or a radical photoinitiator can comprise Irgacure 2959 (Sigma-Aldrich). Photoinitiators are known to the skilled person. In an aspect, a radical photoinitiator can comprise about 0.1% w/v to about 2.0% w/v, or can comprise about 0.5% w/v.

In an aspect, a disclosed method can comprise using a poly(methyl methacrylate) (PMMA) leaching method. PMMA leaching methods are known to the art (see, for example, Zeng Y, et al. (2020) Adv Mater. 32:e1906022)).

In an aspect, about 10 μL to about 50 μL of the mixture was added into a mold, or about 20 μL of the mixture was added into a mold. Molds are known to the skilled person in the art. In an aspect, a disclosed mold can be polypropylene. In an aspect, a disclosed mold can be cylindrical. In an aspect, a disclosed mold can be a cylindrical polypropylene mold. In an aspect, a disclosed cylindrical mold can have about a 1 mm to about 20 mm diameter, or can have about a 5 mm diameter.

In an aspect, a disclosed mold can comprise microbeads. In an aspect, microbeads can comprise PMMA microbeads (such as, for example, 150-180 μm PMMA microbeads from Cospheric). In an aspect, a disclosed mold can comprise about 5 mg of microbeads, about 10 mg of microbeads, about 15 mg of microbeads, about 20 mg of microbeads, about 25 mg of microbeads, about 30 mg of microbeads, or more than 30 mg of microbeads.

In an aspect, a disclosed mold comprising a disclosed mixture can be UV irradiated. In an aspect, UV irradiation can be for about 5 minutes, about 10 minutes, about 15 minutes, about 20 minutes, about 25 minutes, or more than 25 minutes. In an aspect, UV irradiation can occur at 365 nm. In an aspect, UV irradiation can occur at any wavelength that accommodates a disclosed radical photoinitiator.

In an aspect, removing the PMMA microbeads can comprise an acetone soak. In an aspect, an acetone soak can comprise one or more changes of solvent. In an aspect, an acetone soak can comprise about 1 day, about 2 days, about 3 days, about 4 days, about 5 days, or more than 5 days.

In an aspect, washing and rehydrating the resulting porous hydrogel can comprise washing and rehydrating the hydrogel with deionized water.

In an aspect, a disclosed method can comprise loading adenosine into the disclosed scaffolds. In an aspect, adenosine can be purified adenosine or any adenosine known to the art or disclosed herein.

In an aspect, adenosine can be dissolved in PBS or a similar buffer. In an aspect, loading adenosine can comprise soaking disclosed macroporous scaffolds with a disclosed adenosine solution. In an aspect, soaking can comprise about a 4 hour soak, about a 5 hour soak, about a 6 hour soak, about a 7 hour soak, about an 8 hour soak, about a 9 hour soak, about a 10 hour soak, or more than a 10 hour soak.

In an aspect, a disclosed method can comprise measuring the amount of encapsulated adenosine. In an aspect, measuring the amount of encapsulated adenosine can comprise soaking the scaffolds in an acetate buffer (0.1 M, pH 3.5) for about 1 hour to about 3 hours or about 2 hours to release the loaded adenosine into the buffer. In an aspect, the adenosine content in the buffer can be determined by using a UV/vis spectrophotometer (at absorption wavelength of 260 nm). In an aspect, a standard calibration curve of absorbance vs concentration can be generated and can be used to calculate the concentration of the released adenosine in the released media.

In an aspect, a disclosed method of making a disclosed biomaterial can comprise modifying one or more steps of a disclosed method. Modifying can comprise modifying one or more agents, the amounts of one more agents, the time consumed by one or more steps, or any combination thereof.

Disclosed herein is a method of making a disclosed biomaterial, the method comprising synthesizing polyethylene glycol diacrylate (PEGDA). In an aspect, poly(ethylene glycol) can be acrylated as described in Kar M, et al. (2016) Biomaterials. 77:186-197. In an aspect, polyethylene glycol (PEG, MilliporeSigma, Burlington, MA, Cat #P4338) (10 g, ˜3 mmol) can be dissolved in dry dichloromethane (DCM, 100 mL) at room temperature under argon gas. In an aspect, triethylamine (MilliporeSigma, Cat #471283) (627.3 μL, 4.5 mmol) can be added to the solution. In an aspect, a disclosed reaction mixture can be placed on an ice bath. In an aspect, a disclosed acryloyl chloride (MilliporeSigma, Cat #A24109) (364 μL, 4.5 mmol), which can be dissolved in dry DCM (15 mL), can be added dropwise to the mixture and the reaction was continued for about 12 hr at room temperature. In an aspect, a disclosed reaction mixture can then be passed through Celite 545 (MilliporeSigma, Cat #1026931000) and can be concentrated using a rotary evaporator. In an aspect, a disclosed product can be precipitated in excess chilled diethyl ether and can be filtered using Whatman filter paper. In an aspect, a disclosed resultant PEGDA can be dried overnight under vacuum and can be purified by using Sephadex G-25 column (GE Healthcare, Chicago, IL) followed by lyophilization. In an aspect, a disclosed product can be characterized by a combination of FTIR and 1HNMR spectroscopy. In an aspect, a disclosed FTIR spectra can show peaks at 1725 cm⁻¹ corresponding to the ester C═O stretching frequency and can confirm the introduction of acrylate groups in PEGDA via ester bond formation. In an aspect, a disclosed diacrylation in PEGDA can be further evident from 1HNMR as the presence of peaks at 5.81, 6.31, and 6.42 ppm corresponding to vinyl protons of acrylate groups.

Disclosed herein is a method of making a disclosed biomaterial, the method comprising synthesizing N-acryloyl-6-aminocaproic acid (A6ACA). In an aspect, a disclosed A6ACA can be synthesized as described in Phadke A, et al. (2012) Proc Natl Acad Sci USA. 109:4383-4388. In an aspect, 6-Aminocaproic acid (6ACA, MilliporeSigma, Cat #A7824) (13.1 g, 0.1 M) and sodium hydroxide (4.4 g, 0.11 M) can be dissolved in 80 mL water. In an aspect, a disclosed solution can be placed over an ice bath and acryloyl chloride (˜10 g, 0.11 M) dissolved in 15 mL dry tetrahydrofuran (THF) can be added to the 6ACA solution dropwise. In an aspect, the pH of the reaction mixture can be maintained at ˜7.8 during the addition of acryloyl chloride by using 2.5 M NaOH solution. After the reaction, the pH of the mixture can be gradually decreased to ˜3.0 by adding 5 M hydrochloric acid. The product can be then extracted using ethyl acetate, can be concentrated over anhydrous sodium sulfate, and can be precipitated in chilled n-hexane. In an aspect, the product, A6ACA, can be filtered through Whatman filter paper and can be dried overnight under vacuum at 45° C. In an aspect, the product can be characterized by FTIR and/or 1HNMR spectroscopy. In an aspect, a FTIR spectrum can show peaks at 1655 cm⁻¹ and 1543 cm⁻¹ corresponding to the amide C═O and N—H stretching frequencies, respectively. In an aspect, formation of A6ACA can be eviden from 1HNMR as the spectrum showed peaks at 5.74 ppm, 6.18 ppm, and 6.26 ppm corresponding to vinyl protons of acrylamide group.

Disclosed herein is a method of making a disclosed biomaterial, the method comprising fabricating macroporous hydrogel. In an aspect, a disclosed PEGDA-6ACA macroporous hydrogels containing 3-acrylamido phenylboronic acid (3-APBA, MilliporeSigma, Cat #771465) (PEGDA-6ACA-PBA) can be fabricated using a poly(methyl methacrylate) (PMMA) bead leaching method. In an aspect, PEGDA (10% w/v), 3-APBA (1 M), A6ACA (0.5 M) and Irgacure 2959 (MilliporeSigma, Cat #410896, 0.5% w/v) can be dissolved in 20:80 water:ethanol mixture. In an aspect, 20 μL of the mixture can be added into a cylindrical polypropylene mold (˜5 mm in diameter) packed with ˜20 mg of PMMA microbeads (150 μm-180 μm, Cospheric, Santa Barbera, CA). In an aspect, a disclosed mixture can be photopolymerized by UV light irradiation for about 10 min. In an aspect, a disclosed resulting hydrogel network can be embedded with the PMMA beads and can be incubated in acetone for 3 days to remove the beads with frequent changes of solvent. In an aspect, a disclosed macroporous hydrogel can be washed and rehydrated with deionized water. FTIR spectrum of the freeze-dried hydrogel can show a peak at 1725 cm⁻¹ corresponding to the ester C—O stretching frequency indicating the presence of PEGDA. In an aspect, presence of phenylboronic acid (PBA) moieties can be confirmed via 1HNMR spectroscopy. In an aspect, for NMR measurements, freshly prepared porous hydrogels can be thoroughly washed with DI water to remove unreacted precursors and freeze-dried. In an aspect, the freeze-dried samples can be minced and can be fully suspended in D2O by adding 5 M NaOH solution in D2O. In an aspect, the 1HNMR spectrum can be recorded using a 500 MHz Varian spectrometer and can show peaks at 7.07-7.27 ppm corresponding to aromatic protons of PBA. In an aspect, the PBA content in the hydrogel can be quantified by using UV/Vis spectrophotometer. Towards this, in an aspect, the macroporous hydrogels can be dissociated in 5 M NaOH solution in water. The PBA content in the solution can be determined by using a UV/Vis spectrophotometer at an absorption wavelength of 255 nm. In an aspect, a standard calibration curve of absorbance vs concentration can be generated using free 3-APBA solutions of known concentrations (21.25 μg/mL to 170 μg/mL) and can be used to calculate PBA content. The estimated value can indicate the PBA constituted over 45% of the dry weight of hydrogels (e.g., which can further indicated that almost 93±3% of 3-APBA can be incorporated into the hydrogel network. In an aspect, macroporous hydrogels without 3-APBA (PEGDA-6ACA) can be prepared similarly and can be used as controls for animal studies. In an aspect, for sterilization, hydrogels can be soaked in 70% ethanol for 6 hr and can be washed extensively in phosphate buffered saline (PBS) for 3-4 days.

In an aspect, a disclosed method can comprise adenosine loading. In an aspect, to load adenosine (MilliporeSigma, Cat #A4036) into the macroporous hydrogel (PEGDA-6ACA-PBA), hydrogel discs can be incubated in adenosine solution in PBS at 6 mg/mL for about 6 hr at 37° C. and can be washed thoroughly to remove any unbound adenosine. In an aspect, to measure the amount of adenosine loaded, the discs can be soaked in acetate buffer (0.1 M, pH 3.5) for about 2 hr to release the adenosine into the buffer. In an aspect, the adenosine content in the buffer can be determined by using a UV/Vis spectrophotometer at wavelength of 260 nm. In an aspect, a standard calibration curve of absorbance vs. concentration can be generated using adenosine solutions of known concentrations (3.9 μg/mL-125 μg/mL) and can be used to calculate the adenosine concentration.

In an aspect, for example, a disclosed biomaterial can comprise from about 1 mg/kg to about 30 mg/kg of ADO to body weight. In an aspect, for example, a disclosed biomaterial can comprise from about 1 mg/kg to about 5 mg/kg of ADO to body weight, from about 5 mg/kg to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed biomaterial can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg of ADO to body weight.

In an aspect, for example, a disclosed therapeutically effective dose of ADO can comprise from about 1 mg/kg to about 30 mg/kg ADO to body weight. In an aspect, for example, a disclosed therapeutically effective dose of ADO can comprise from about 1 mg/kg to about 5 mg/kg ADO to body weight, from about 5 mg/kg ADO to about 10 mg/kg ADO to body weight, from about 10 mg/kg to about 15 mg/kg ADO to body weight, from about 15 mg/kg to about 20 mg/kg ADO to body weight, from about 20 mg/kg to about 25 mg/kg ADO to body weight, or from about 25 mg/kg to about 30 mg/kg ADO to body weight. In an aspect, for example, a disclosed therapeutically effective dose of ADO can comprise about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg ADO to body weight.

### E. Miscellaneous

Disclosed herein is a method of treating, preventing, and/or mitigating pain in a subject, the method comprising administering to a subject having pain a biomaterial comprising a therapeutically effective amount of adenosine. In an aspect, a disclosed biomaterial can comprise a macroporous hydrogel, macroporous scaffolds, or a hydrogel comprising macroporous scaffolds. In an aspect, a disclosed biomaterial can comprise conjugated 3-APBA. In an aspect, administering can comprise implanting the biomaterial at the site of the subject's pain. In an aspect, a disclosed subject can be a non-human mammal or a human. In an aspect, a disclosed pain can comprise acute pain or chronic pain, post-surgical pain, cancer pain, injury pain, and/or pain associated with bone fracture. In an aspect, a disclosed method can further comprise improving the subject's ability to move. In an aspect, a disclosed method can further comprise attenuating A1R activation and/or A3R activation in the subject.

Disclosed herein is a method of treating, preventing, and/or mitigating pain in a subject having one or more bone fractures, the method comprising implanting a biomaterial comprising a therapeutically effective amount of adenosine at one or more bone fractures in a subject; wherein the subject's pain associated with the one or more bone fractures is reduced. In an aspect, a disclosed biomaterial can comprise a macroporous hydrogel, macroporous scaffolds, or a hydrogel comprising macroporous scaffolds. In an aspect, a disclosed biomaterial can comprise conjugated 3-APBA. In an aspect, a disclosed method can further comprise implanting an orthopedic implant at the site of one or more of the subject's bone fractures. In an aspect, a disclosed subject can be a non-human mammal or a human. In an aspect, the one or more disclosed bone fractures can be the result of an injury, a surgery, or a physical trauma. In an aspect, the one or more disclosed bone fractures can be the result of a non-trauma, a disease, or a disorder. In an aspect, the subject can have an opioid dependence. In an aspect, a disclosed method can further comprise improving the subject's ability to move. In an aspect, a disclosed method can further comprise promoting bone fracture healing in the subject. In an aspect, a disclosed method can further comprise repairing or partially repairing a skeletal defect in the subject. In an aspect, a disclosed method can further comprise enhancing the innate ability of bone repair tissue to repair bone in the subject. In an aspect, a disclosed method can further comprise reducing bone degeneration in the subject, promoting bone regeneration in the subject, or a combination thereof. In an aspect, a disclosed method can further comprise promoting osteoblastogenesis in and around the area of the one or more bone fractures, decreasing osteoclastogenesis in and around the area of the bone fracture, or a combination thereof. In an aspect, a disclosed method can further comprise attenuating A1R activation and/or A3R activation in the subject. In an aspect, a disclosed method can further comprise decreasing activation of one or more dorsal root ganglions (DRGs).

Disclosed herein is a method of treating a subject in need of non-opioid analgesia, the method comprising administering to a subject a biomaterial comprising a therapeutically effective amount of adenosine; wherein the subject's pain is reduced. In an aspect, a disclosed biomaterial can comprise a macroporous hydrogel, macroporous scaffolds, or a hydrogel comprising macroporous scaffolds. In an aspect, a disclosed biomaterial can comprise conjugated 3-APBA. In an aspect, a disclosed subject can have one or more bone fractures. In an aspect, administering the biomaterial can comprise implanting the biomaterial at the site of the one or more factures. In an aspect, one or more disclosed bone fractures can be the result of an injury, a surgery, or a physical trauma. In an aspect, one or more disclosed bone fractures can be the result of a non-trauma, a disease, or a disorder. In an aspect, a disclosed subject can have an opioid dependence. In an aspect, a disclosed method can further comprise improving the subject's ability to move. In an aspect, a disclosed method can further comprise promoting bone fracture healing in the subject. In an aspect, a disclosed method can further comprise repairing or partially repairing a skeletal defect in the subject. In an aspect, a disclosed method can further comprise enhancing the innate ability of bone repair tissue to repair bone in the subject. In an aspect, a disclosed method can further comprise reducing bone degeneration in the subject, promoting bone regeneration in the subject, or a combination thereof. In an aspect, a disclosed method can further comprise promoting osteoblastogenesis in and around the area of the one or more bone fractures, decreasing osteoclastogenesis in and around the area of the one or more bone fractures, or a combination thereof. In an aspect, a disclosed method can further comprise attenuating A1R activation in the subject. In an aspect, a disclosed method can further comprise decreasing activation of one or more dorsal root ganglions (DRGs).

### EXAMPLES

Bone fracture is a common injury associated with sports, accidents, or falls. These injuries and post-surgical intervention to repair bone are accompanied by acute pain (Chang K, et al. (2017) JAMA. 318:1661-1667), which gradually diminishes as bone heals (Corrales L A, et al. (2008) J Bone Joint Surg Am. 90:1862-1868). The management of bone injuries generally involve stabilization, surgery, rehabilitation, administration of analgesics such as nonsteroidal anti-inflammatory drugs (NSAIDs) or opioids and allowing the injured tissue to heal (Chang K, et al. (2017) JAMA. 318:1661-1667; Duan X, et al. (2012) Cochrane Database Syst Rev. 1: CD008241; Gosler M W, et al. (2012) Cochrane Database Syst Rev. 1: CD008832). Despite the regenerative capacity of bone, some fractures suffer delayed healing or nonunion, typically requiring repeated surgical interventions, which is a challenging clinical problem and leads to long-term morbidity and chronic pain (Hak D J, et al. (2014) Injury. 45 Suppl 2:S3-S7; Mills L A, et al. (2017) Acta Orthop. 88:434-439). The incidence of nonunion varies from 1.9% to 30% depending upon the severity of fracture, comorbidities, and lifestyle habits (Hak D J, et al. (2014) Injury. 45 Suppl 2:S3-S7; Mills L A, et al. (2017) Acta Orthop. 88:434-439). In addition, the ongoing pain associated with impaired healing often transition to chronic and maladaptive pain (Chartier S R, et al. (2014) Pain. 155:2323-2336; Glare P, et al. (2019) Lancet. 393:1537-1546).

Traumatic injury and surgery are accompanied by acute pain that is considered part of the normal healing process whereby tissue damage elicits hypersensitivity to promote guarding of the injured tissue (Ahuja C S, et al. (2017) Nat Rev Dis Primers. 3:17018; Chang A K, et al. (2017) JAMA 318:1661-1667; Abou-Setta A M, et al. (2011) Ann Intern Med. 155:234-245; Jeschke M G, et al. (2020) Nat Rev Dis Primers. 6:11; van Tulder M, et al. (2007) Lancet. 369:1815-1822; Wick E C, et al. (2017) JAMA Surg. 152:691-697). During bone healing, peripheral nerves play an important role as extensive sensory and sympathetic nerve sprouting occur upon fracture injury (Yasui M, et al. (2012) Eur J Pain. 16:953-965), and inhibition of their formation affects fracture healing (Li Z, et al. (2019) J Clin Invest. 129:5137-5150). Paradoxically, the growth of peripheral nerves also contributes to pain during normal repair and chronic conditions involving nonunion of bone defects (Yasui M, et al. (2012) Eur J Pain. 16:953-965; Chartier S R, et al. (2014) Pain. 155:2323-2336). The standard treatment for skeletal fracture accompanied by pain involves stabilization of the fractured bone, rehabilitation, and administration of nonsteroidal anti-inflammatory drugs (NSAIDs) and opiates to control the pain (Duan X, et al. (2012) Cochrane Database Syst Rev. 1: CD008241; Gosler M W, et al. (2012) Cochrane Database Syst Rev. 1: CD008832; Chang A K, et al. (2017) JAMA. 318 (17): 1661-1667; Hoyt B W, et al. (2015) Curr Trauma Rep. 1 (1): 50-60). Because bone trauma elicits one of the most severe pain, adequate management with analgesics is required before the pain gradually recedes as healing progresses (Prasad A, et al. (2018) J Burn Care Res. 39:433-439; Majuta L A, et al. (2015) Pain. 156:157-165; Corrales L A, et al. (2008) J Bone Joint Surg Am. 90:1862-1868).

While NSAIDs and opioids are effective and commonly prescribed to manage acute pain, their use in patients with fractures has been associated with several substantial drawbacks. Studies have shown NSAIDs could delay bone healing (Cottrell J, et al. (2010) Pharmaceuticals (Basel). 3:1668-1693; Geusens P, et al. (2013) Curr Opin Rheumatol. 25:524-531; Pountos I, et al. (2012) ScientificWorldJournal, 2012:606404; Wheatley B M, et al. (2019) J Am Acad Orthop Surg. 27:e330-e336), and post-operative use of opioids for acute pain increases risk of nonunions and long-term use that may contribute to opioid epidemic (Hsu J R, et al. (2019) J Orthop Trauma. 33:e158-e182; Hooten W M, et al. (2015) Mayo Clin Proc. 90:850-856; Martin B C, et al. (2011) J Gen Intern Med. 26:1450-1457; Zura R, et al. (2016) JAMA Surg. 151:e162775). Post-operative use of opiates for acute pain increases the risk of long-term use (Hsu J R, et al. (2019) J Orthop Trauma. 33 (5):e158-e182). Moreover, a substantial number of fractures result in delayed healing and non-unions that contribute to chronic pain and the need for long-term use of opiates (Antonova E, et al. (2013) BMC Musculoskelet Disord. 14:42).

In addition to the abuse and addiction, long-term use of opiates could lead to organ damage and demotivation and can interfere with functional activity and the ability to return to work (Kidner C L, et al. (2009) J Bone Joint Surg Am. 91 (4): 919-927; Sullivan M D, et al. (2013) Pain. 154 Suppl 1:S94-100; Lee M, et al. (2011) Pain Physician. 14 (2): 145-161). The societal toll of the opioid epidemic in the United States is manifested by studies showing that in 2019 alone, ˜10.3 million people misused prescription opioids while 49,860 people died from overdose (What is the U.S. Opioid Epidemic?). Despite reduction of opioid prescriptions by orthopedic surgeons in recent years, the opioid crisis continues in the United States, and even worsened during the COVID-19 pandemic (Acuna A J et al. (2021) J Am Acad Orthop Surg Glob Res Rev. 5 (5):e21 (2021); Holland K M et al. (2021) JAMA Psychiatry. 78:372-379). For instance, studies have shown that 6% of patients receiving short-term opiate prescription progress to long-term use (Hooten W M, et al. (2015) Mayo Clin Proc. 90 (7): 850-856), and up to half of those who take opiates for at least 3 months remain on opioids even after 5 years and are likely to become lifelong users (Martin B C, et al. (2011) J Gen Intern Med. 26 (12): 1450-1457). Effective strategies to manage acute pain and prevention of chronic pain are urgently needed and the development of novel therapies for patients with orthopedic injuries such as fracture pain is highly warranted.

Considering the problems associated with current analgesics and lack of osteoanabolic treatments to promote bone tissue regeneration, new approaches that simultaneously manage pain and promote healing would address a significant clinical need.

To this end, a new therapeutic solution—the localized administration of adenosine and its efficacy to mitigate both pain and promote tissue regeneration following bone injury or orthopedic surgery—is described below. Accordingly, the studies described herein utilized a biomaterial-based approach for sustained in situ delivery of adenosine (which simultaneously targeted multiple adenosine receptor subtypes) in a model of fracture injury to examine its therapeutic effect to mitigate pain, promote healing, and improve function.

The Examples that follow are illustrative of specific embodiments of the invention, and various uses thereof. They set forth for explanatory purposes only and are not to be taken as limiting the invention.

### Materials and Methods for Examples 1-6

Whether the purine molecule, adenosine, could simultaneously alleviate pain and promote healing in a mouse model of tibial fracture by targeting different adenosine receptor subtypes in different cell populations was examined. The data demonstrated that local delivery of adenosine inhibited nociceptive activity of peripheral neurons through activation of adenosine A1 receptor (ADORA1) and mitigated pain as demonstrated by weight bearing and open field movement. Concurrently, localized administration of adenosine at the fracture site also promoted osteogenic differentiation of mesenchymal stromal cells through adenosine A2B receptor (ADORA2B) and improved bone healing shown by histological analyses and microCT imaging. This study demonstrated the unique functional properties of adenosine along with a material-assisted local delivery of adenosine as a feasible translatable therapeutic strategy to treat bone trauma and associated pain.

### 1. Study Design

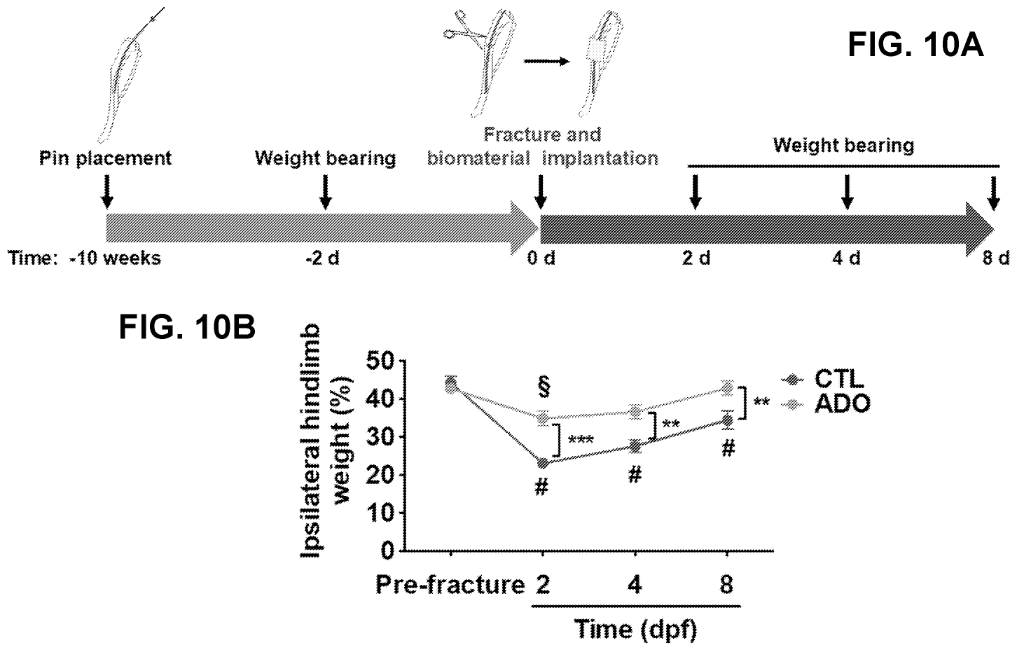

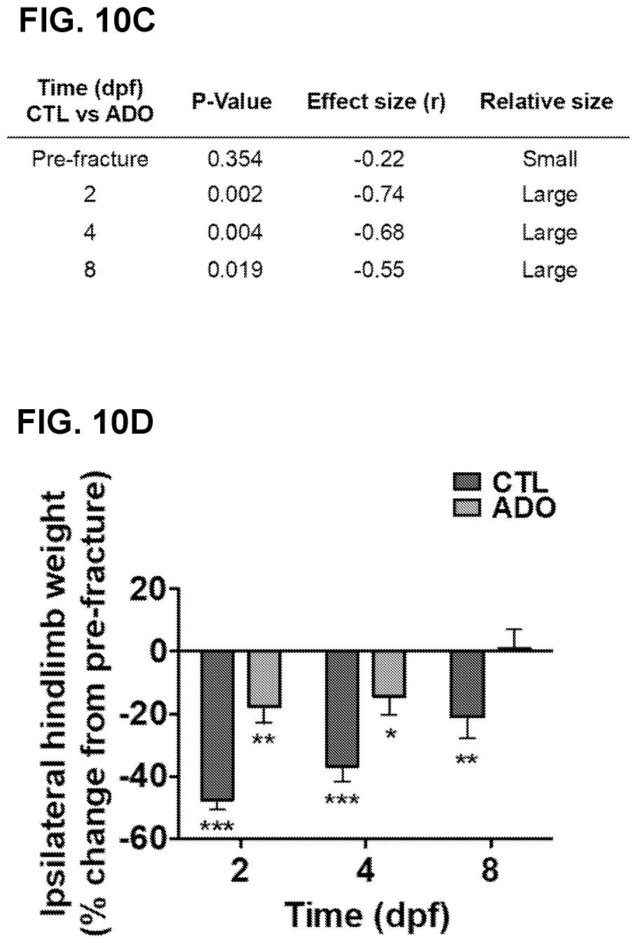

The aim of the experiments described herein was to examine the effectiveness of local delivery of adenosine in tibial bone injury to concurrently attenuate fracture/post-surgical pain while promoting healing. All animal work was performed in compliance with the National Institutes of Health (NIH) and institutional guidelines, and the fracture surgery and biomaterial implantation protocols approved by the Institutional Animal Care and Use Committee at Duke University (Protocol Registry Number A151-20-07) were followed. C57BL/6J mice were used to study fracture nociception and the effect of local delivery of adenosine to mitigate pain and promote bone regeneration. Lepr-cre; tdTomato reporter mice were used for tracing of LepR(+) lineage cells after fracture. To determine the effect of adenosine on pain and healing, fracture surgeries were performed and treated immediately with PEGDA-6ACA-PBA macroporous hydrogels loaded with adenosine as treatment group or without adenosine as control group (Zeng Y, et al. (2020) Adv Mater. 32:e1906022). Behavioral tests for pain and limb function were assessed by weight bearing and open field locomotion. A priori power analysis was used to estimate the sample size requiring a statistical power (1−β) of 80% at the significance level of α=5%. On the basis of the power calculation, the indicated number was 8 animals per group for fracture pain and 4 animals per group for fracture healing. Ten animals per group were used to study the effect of adenosine on fracture pain, and 5 animals per group were used to examine the effect of adenosine on fracture healing. The exclusion criteria for experiments were animals that died due to the surgical procedures or shift of the intramedullary pin identified after experimental endpoint post-mortem. No randomization was used and researchers were not blinded. Measurement of adenosine in the circulation was performed by using an adenosine assay. The in vitro functional activity of DRG neurons was assessed by calcium and membrane potential imaging and the expression of proteins were assessed by histology or immunofluorescence imaging, and gene expression by RT-qPCR. Fracture healing was analyzed by microCT and histology. The study design and timeline of animal procedures (pin placement, surgery, therapeutic intervention with and without adenosine, and behavioral tests) are illustrated in FIG. 10A (weight bearing) and FIG. 10A (open field locomotion). The sample number, statistical methods, and timepoints for each experiment are provided in the figure legends.

### 2. Fracture Surgery and Therapeutic Intervention

In all experiments, 12-week-old to 16-week-old C57BL/6J male mice (Jackson Laboratory, Bar Harbor, ME) and Lepr-cre; tdTomato reporter mice were used. The average mouse weight was 25 grams and mice were housed in groups of four to five, and kept on a 12-hr light-dark cycle with lights off at 1800 hr. All animals had ad libitum access to chow (PMI LabDiet 5001) and water. Fracture surgeries were performed as previously described (Zeng Y, et al. (2020) Adv Mater. 32:e1906022). Animals were anesthetized by isoflurane and injected with 1 mg/kg buprenorphine SR-LAB (ZooPharm, Laramie, WY). Each mouse was then placed in a supine position with the right tibia disinfected. After skin incision and reflection, proximal to the right knee, a 0.7-mm pin was inserted from the tibial plateau through the medullary cavity to stabilize the tibia, and a cut was made at the tibial midshaft to induce a transverse fracture. Bupivacaine (0.5%; Hospira, Lake Forest, IL) at ˜1 mg/kg body weight was applied at the surgical site following wound closure using 2 reflex clips (5 mm). For animals treated with control and adenosine-loaded macroporous hydrogels, macroporous hydrogels measuring approximately 5 mm (length)×3 mm (width)×1 mm (thick) containing either 1.05±0.1 mg adenosine (ADO) or no adenosine (CTL) were implanted adjacent to the fractures immediately. For behavioral tests, fracture surgery was delayed until 10 weeks after the pin placement to avoid interference from pain originating from sites other than the fracture site, as the pin insertion alone has been shown to induce pain (Majuta L A, et al. (2015) Pain. 156:157-165). The animals were treated with the macroporous hydrogels with or without adenosine immediately following the fracture. In the cohorts for weight bearing and open field activity measurements, 1 animal from the control group and 1 animal from the treatment group were euthanized due to shift of the inserted pin following fracture.

### 3. Synthesis and Characterization of Macroporous Hydrogels

a. Synthesis of Polyethylene Glycol Diacrylate (PEGDA)

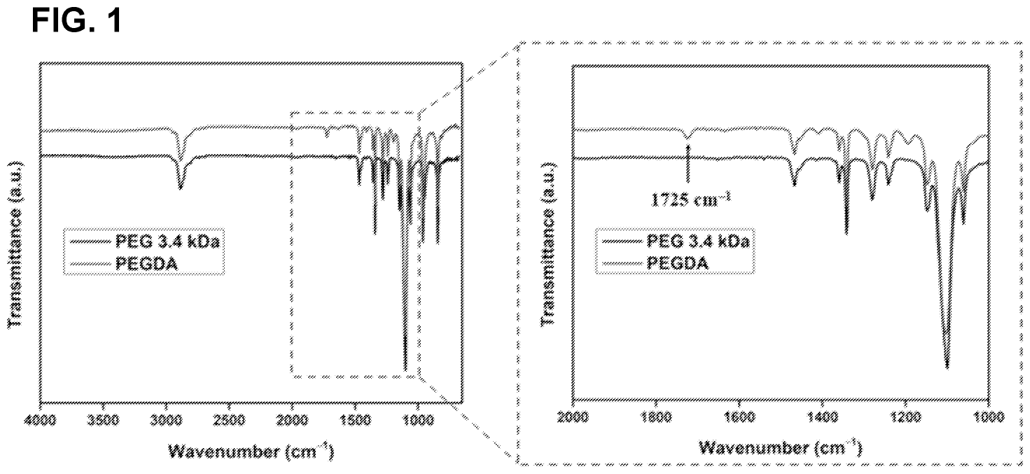

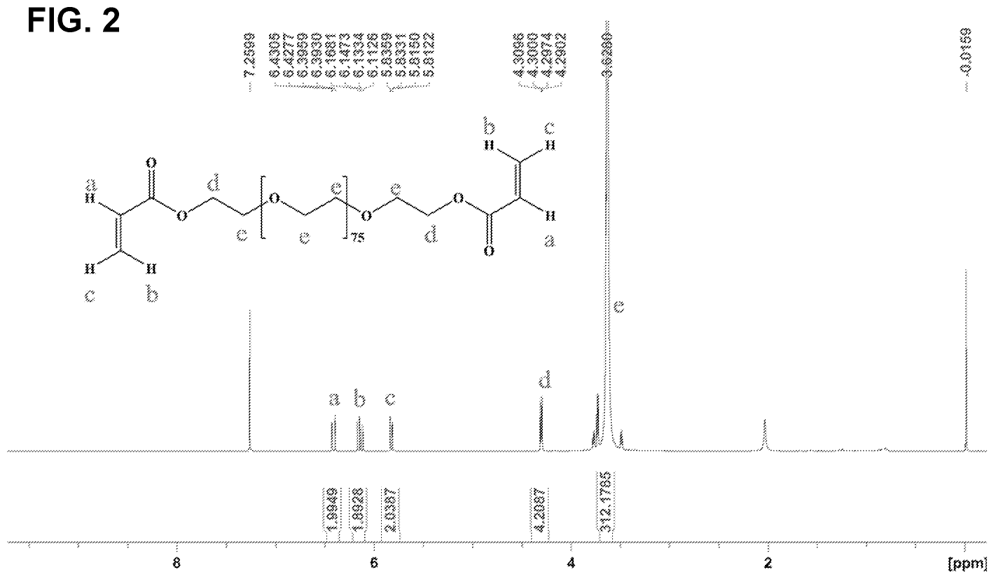

Poly(ethylene glycol) was acrylated as described elsewhere (Kar M, et al. (2016) Biomaterials. 77:186-197). In brief, polyethylene glycol (PEG, MilliporeSigma, Burlington, MA, Cat #P4338) (10 g, ˜3 mmol) was dissolved in dry dichloromethane (DCM, 100 mL) at room temperature under argon gas. Triethylamine (MilliporeSigma, Cat #471283) (627.3 μL, 4.5 mmol) was added to the solution. The reaction mixture was then placed on an ice bath. Acryloyl chloride (MilliporeSigma, Cat #A24109) (364 μL, 4.5 mmol), dissolved in dry DCM (15 mL), was added dropwise to the mixture and the reaction was continued for about 12 hr at room temperature. The reaction mixture was then passed through Celite 545 (MilliporeSigma, Cat #1026931000) and concentrated using a rotary evaporator. The product was precipitated in excess chilled diethyl ether and filtered using Whatman filter paper. The resultant PEGDA was dried overnight under vacuum and purified by using Sephadex G-25 column (GE Healthcare, Chicago, IL) followed by lyophilization. The product was characterized by a combination of FTIR and ¹HNMR spectroscopy. The FTIR spectra showed peaks at 1725 cm⁻¹ corresponding to the ester C═O stretching frequency thus confirming the introduction of acrylate groups in PEGDA via ester bond formation (FIG. 1). The diacrylation in PEGDA was further evident from 1HNMR as the presence of peaks at 5.81, 6.31, and 6.42 ppm corresponding to vinyl protons of acrylate groups (FIG. 2).

b. Synthesis of N-Acryloyl-6-Aminocaproic Acid (A6ACA)

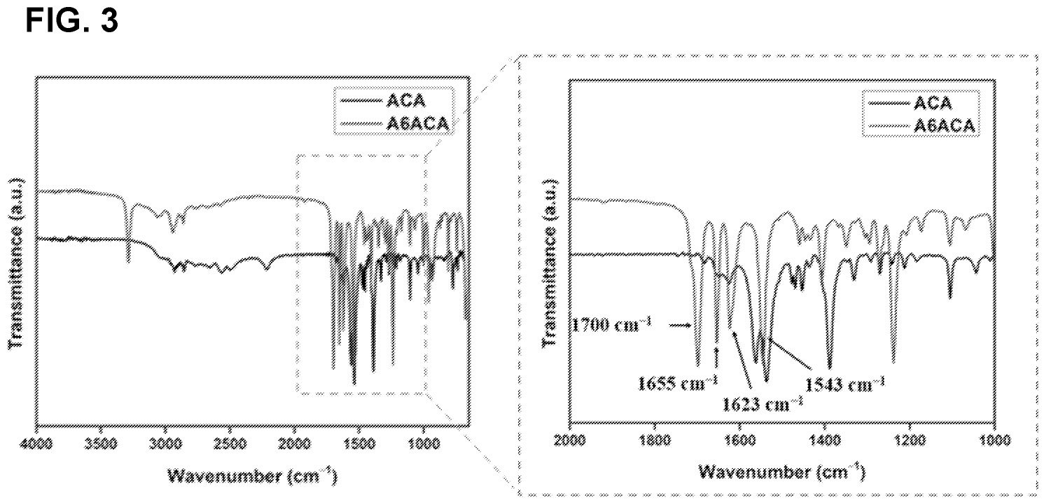

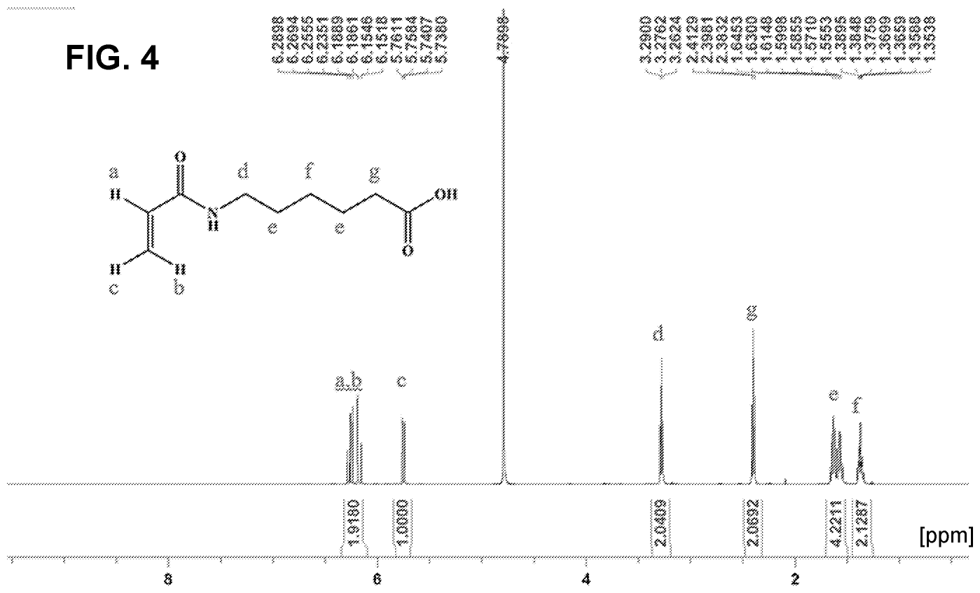

A6ACA was synthesized as described earlier (Phadke A, et al. (2012) Proc Natl Acad Sci USA. 109:4383-4388). 6-Aminocaproic acid (6ACA, MilliporeSigma, Cat #A7824) (13.1 g, 0.1 M) and sodium hydroxide (4.4 g, 0.11 M) were dissolved in 80 mL water. The solution was placed over an ice bath and acryloyl chloride (˜10 g, 0.11 M) dissolved in 15 mL dry tetrahydrofuran (THF) was added to the 6ACA solution dropwise. The pH of the reaction mixture was maintained at ˜7.8 during the addition of acryloyl chloride by using 2.5 M NaOH solution. After the reaction, the pH of the mixture was gradually decreased to ˜3.0 by adding 5 M hydrochloric acid. The product was then extracted using ethyl acetate, concentrated over anhydrous sodium sulfate, and precipitated in chilled n-hexane. The product, A6ACA, was filtered through Whatman filter paper and dried overnight under vacuum at 45° C. The product was characterized by FTIR and 1HNMR spectroscopy. The FTIR spectrum showed peaks at 1655 cm⁻¹ and 1543 cm⁻¹ corresponding to the amide C═O and N—H stretching frequencies, respectively (FIG. 3). Formation of A6ACA was further evident from 1HNMR as the spectrum showed peaks at 5.74, 6.18 and 6.26 ppm corresponding to vinyl protons of acrylamide group (FIG. 4).

c. Macroporous Hydrogel Fabrication

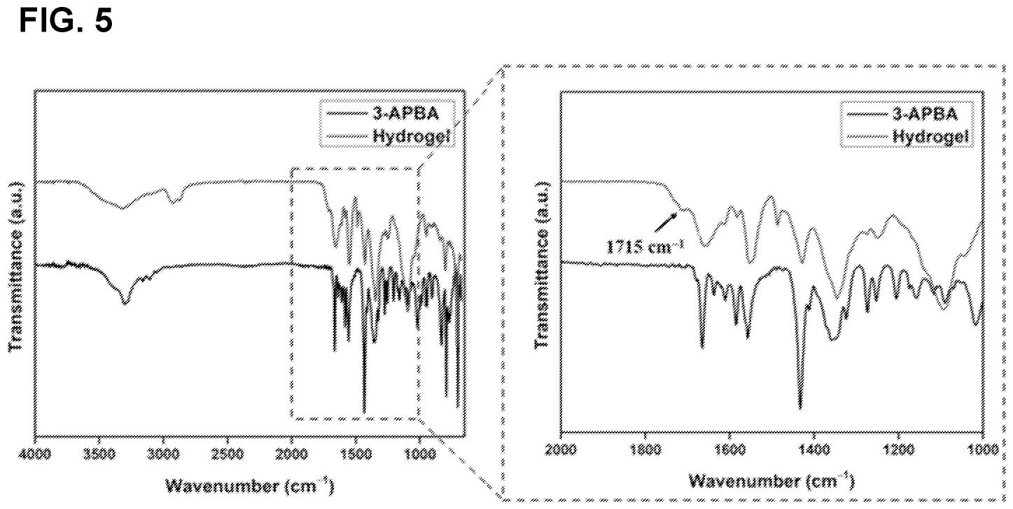

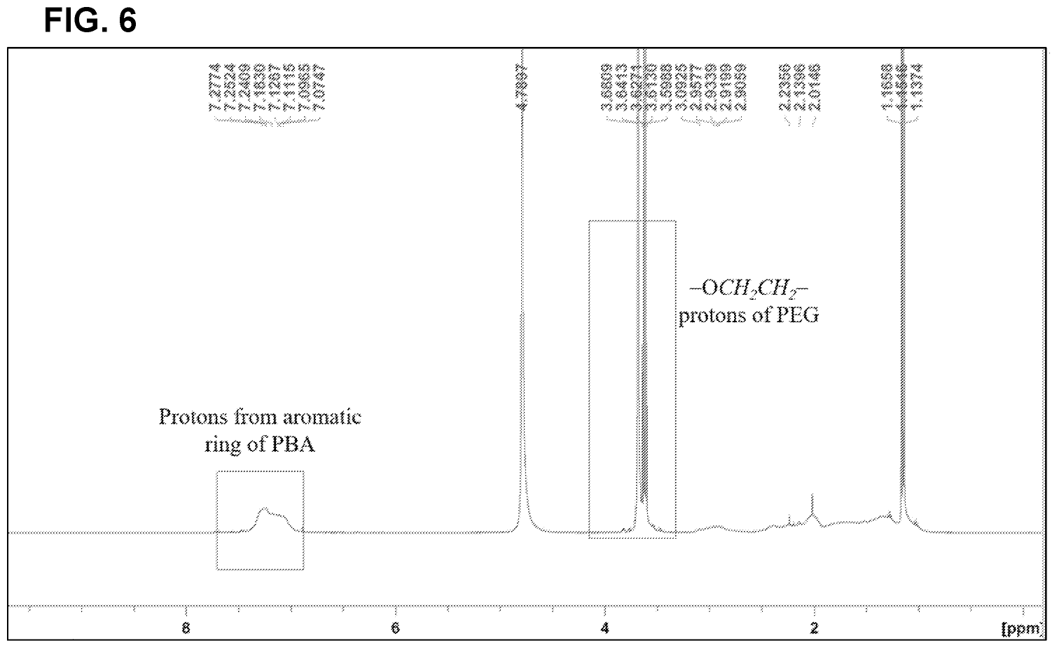

The PEGDA-6ACA macroporous hydrogels containing 3-acrylamido phenylboronic acid (3-APBA, MilliporeSigma, Cat #771465) (PEGDA-6ACA-PBA) were fabricated using a poly(methyl methacrylate) (PMMA) bead leaching method (Zeng Y, et al. (2020) Adv Mater. 32:e1906022). PEGDA (10% w/v), 3-APBA (1 M), A6ACA (0.5 M) and Irgacure 2959 (MilliporeSigma, Cat #410896, 0.5% w/v) were dissolved in 20:80 water:ethanol mixture. 20 μL of the mixture was added into a cylindrical polypropylene mold (˜5 mm in diameter) packed with ˜20 mg of PMMA microbeads (150 μm-180 μm, Cospheric, Santa Barbera, CA). The mixture was photopolymerized by UV light irradiation for about 10 min. The resulting hydrogel network embedded with the PMMA beads was incubated in acetone for 3 days to remove the beads with frequent changes of solvent. The macroporous hydrogel was washed and rehydrated with deionized water. FTIR spectrum of the freeze-dried hydrogel showed a peak at 1725 cm⁻¹ corresponding to the ester C—O stretching frequency indicating the presence of PEGDA (FIG. 5). Presence of phenylboronic acid (PBA) moieties was confirmed via ¹HNMR spectroscopy. For NMR measurements, freshly prepared porous hydrogels were thoroughly washed with DI water to remove unreacted precursors and freeze-dried. The freeze-dried samples were minced and fully suspended in D₂O by adding 5 M NaOH solution in D₂O. The ¹HNMR spectrum, recorded using a 500 MHz Varian spectrometer, showed peaks at 7.07 ppm-7.27 ppm corresponding to aromatic protons of PBA (FIG. 6). The PBA content in the hydrogel was quantified by using UV/Vis spectrophotometer. Towards this, the macroporous hydrogels were dissociated in 5 M NaOH solution in water. The PBA content in the solution was determined by using a UV/Vis spectrophotometer at an absorption wavelength of 255 nm. A standard calibration curve of absorbance vs concentration was generated using free 3-APBA solutions of known concentrations (21.25 μg/mL to 170 μg/mL) and used to calculate PBA content. The estimated value indicated that the PBA constituted over 45% of the dry weight of hydrogels which further indicated that almost 93±3% of 3-APBA was incorporated into the hydrogel network. Macroporous hydrogels without 3-APBA (PEGDA-6ACA) was prepared similarly and used as controls for animal studies. For sterilization, the hydrogels were soaked in 70% ethanol for 6 hr and washed extensively in phosphate buffered saline (PBS) for 3-4 days.

d. Adenosine Loading

To load adenosine (MilliporeSigma, Cat #A4036) into the macroporous hydrogel (PEGDA-6ACA-PBA), hydrogel discs were incubated in adenosine solution in PBS at 6 mg/mL for about 6 hr at 37° C. and washed thoroughly to remove any unbound adenosine. To measure the amount of adenosine loaded, the discs were soaked in acetate buffer (0.1 M, pH 3.5) for about 2 hr to release the adenosine into the buffer. The adenosine content in the buffer was determined by using a UV/Vis spectrophotometer at wavelength of 260 nm. A standard calibration curve of absorbance vs. concentration was generated using adenosine solutions of known concentrations (3.9 μg/mL-125 μg/mL) and used to calculate the adenosine concentration.

e. Adenosine Release

To determine the release profile of adenosine, adenosine loaded macroporous hydrogels were incubated in α-MEM (ThermoFisher Scientific, Waltham, MA, Cat #12561056) containing 10% (v/v) fetal bovine serum (FBS) at 37° C. At predetermined time intervals, 20 vol % of the medium was removed and supplemented with fresh medium. The concentration of adenosine in the released medium was determined via UV/Vis absorption spectroscopy using a standard calibration curve as described above.

### 4. Behavioral Tests

a. Weight Bearing

Static weight bearing was measured using an incapacitance meter (IITC Life Science, Woodland Hills, CA). Prior to measurements, mice were trained on the incapacitance meter for 5 days (10 min/day). Data were collected when the animal stood in an upright position and facing front, without noticeable weight shift, lifting or offloading a limb, or turning the head. Each animal was tested for 5 to 6 trials and the weight of both hind limbs was recorded. Weight bearing of the fractured limb (i.e., ipsilateral, right limb) was expressed as a percentage of the total weight borne by the hindlimbs calculated by: (weight of ipsilateral hindlimb/total weight of both hindlimbs)*100.

b. Open Field Activity

Open field activity was performed at Duke Mouse Behavioral and Neuroendocrine Core similar to ref. (Tatem K S, et al. (2014) J Vis Exp. 91:51785). Mice were acclimated to the room for a day prior to testing. Mice were placed individually into the VersaMax open field activity monitoring system with clear acrylic test chambers (40×40×30 cm) and a grid of infrared photobeams containing photocells and sensors to collect and analyze vertical, horizontal, and stereotypic activity (AccuScan Instruments, Columbus, OH). Locomotion was monitored over 60 min at 5 min intervals. The vertical activity (units), vertical movement time (s), ambulatory activity count, ambulatory time (s), total distance traveled (cm), and rest time (s) were determined by the software. Results were expressed as a ratio of post-fracture divided by pre-fracture (baseline) values.

### 5. Histology

For safranin O staining, tibiae were fixed with 4% PFA at 4° C. for 1 day and decalcified using 14% ethylenediaminetetracetic acid (EDTA, pH 7.3) for 2 weeks at 4° C. with constant shaking. The samples were gradually dehydrated using increasing concentrations of ethanol and incubated in Citrisolv (Decon Laboratories, King of Prussia, PA) until equilibrium was reached. Following dehydration, samples were immersed in a mixture of 50% (v/v) Citrisolv and 50% (w/w) paraffin (General Data Healthcare, Cincinnati, OH) for 30 min at 70° C. The samples were embedded in paraffin and 7-μm thick sections were generated using a rotary microtome (Leica Microsystems, Buffalo Grove, IL, RM2255). For immunofluorescence staining of DRG frozen sections, L3-L4 DRG were dissected, fixed with 4% PFA at 4° C. for 2 hr, and incubated in 30% sucrose overnight, embedded in OCT, and 5-μm thick sections were generated by using a cryostat (Leica, CM1850).

### 6. MicroCT

Tibiae were collected, fixed in 4% PFA at 4° C. for 1 day, and rinsed with PBS. The fixed samples were placed in 50-mL centrifuge tubes with styrofoam spacers and loaded into a microcomputed tomography (microCT) scanner (vivaCT 80, Scanco Medical, Wayne, PA). The samples were scanned at 55 keV at a pixel resolution of 10.4 μm. The reconstruction of the images was performed using microCT Evaluation Program V6.6 (Scanco Medical), followed by generation of radiographs and 3D models using microCT Ray V4.0 (Scanco Medical). Total volume (TV), bone volume (BV) per total volume (TV) (% BV/TV), and bone mineral density (BMD) was quantified using the phantom as a reference based on 100 contiguous slices.

### 7. Cell Isolation and In Vitro Culture

a. DRG Neurons

DRG were isolated from mice using a modified version of the previously described protocol (Perner C, et al. (2021) STAR Protoc. 2:100333). Briefly, mice were euthanized and dosed in 70% ethanol to prevent fur contamination. Mice were positioned prone, and an incision was made along the spine from the neck to the base of the tail. Under a dissecting microscope, the surrounding soft tissue of the spinal column was removed from the dorsal side by Friedman-Pearson Rongeurs (Fine Science Tools, Foster City, CA, Cat #16021-14) from the mid thoracic region to the lumbosacral joint. The vertebral bone surrounding the DRG was gently crushed and removed with rongeurs exposing the spinal cord. The DRG were carefully extracted near the foramen between the vertebral levels. L3 and L4 DRG were collected for analyses. Dissected DRG were placed directly into ice-cold DMEM-F12 medium (ThermoFisher, Cat #11320033) for in vitro culture, Trizol (ThermoFisher, Cat #15596018) for PCR analyses, or 4% PFA for histology. For cell culture experiments, collected DRG were digested in digestion solution comprised of collagenase type II (Worthington, Lakewood, NJ, Cat #LS004176) and dispase (1.5 mg/mL each) dissolved in DMEM/F12. DRG were agitated in a digestion solution on an orbital shaker at 60 rpm and 37° C. for 20 minutes and repeated thrice with fresh digestion solution. Next, the digestion solution was replaced with trypsin-EDTA (0.025%) in DMEM/F12 and incubated for an additional 15 min to disrupt the remaining cell-to-cell adhesions. The solution was then replaced with DMEM/F12 and FBS (1:3) to neutralize trypsin. DRG were triturated to create a cell suspension and gently layered onto 15% bovine serum albumin (BSA) solution without mixing and centrifuged for 6 min at 280 g with minimal acceleration and no deceleration to separate non-neuronal cells and debris (Owen D E, et al. (2012) Methods Mol Biol. 846:179-187). The supernatant was carefully aspirated, and the sensory neurons were resuspended in DRG culture medium.

To culture DRG neurons, culture media composed of Neurobasal-A medium (ThermoFisher, Cat #10888022), 2% B27 supplement (ThermoFisher, Cat #17504044), 1% glutamax (ThermoFisher, Cat #35050061), and penicillin/streptomycin (10000 U/mL, 1% v/v, ThermoFisher, Cat #15140122) was used. Cells were treated with or without NGF (200 ng/mL) for 24 hr prior to immunofluorescence imaging. Cells were exposed to NGF (200 ng/mL) for 24 hr (long-term) for calcium and FluoVolt imaging. Cells were cultured on custom-made glass surface with silicone wells. Briefly, cover slides (#1 thickness) were cleaned by agitating in 0.5 M NaOH for 30 minutes followed by subsequent rinsing in dH₂O followed by 100% ethanol and air-dried. Wells were produced in cured polydimethylsiloxane (PDMS, Sylgard 184, Ellsworth Adhesives, Germantown, WI) with a 8-mm biopsy punch and bonding to the cover slides. The wells were coated with poly-lysine by treating overnight with 0.1 mg/mL poly-d-lysine solution, rinsed with sterile water and air-dried. These custom-fabricated wells were stored for up to 7 days prior to use. The wells were treated with laminin (20 μg/mL) for at least 4 hr prior to cell seeding. Laminin solution was aspirated out, incubated with culture medium for 1 hr prior to culturing DRG neurons.

b. MSCs

MSCs were isolated as previously described with some modifications (Shih Y B, et al. (2019) Sci Adv. 5:eaax1387). Briefly, the femurs, tibiae, vertebrae of mice were harvested, crushed with pestle and mortar in harvest buffer (1% v/v FBS in PBS) to release bone marrow (BM) tissue, filtered through a 40-μm cell strainer, and centrifuged at 200 rcf. Cells were seeded in a 24-well plate at a cell density of 1 million cells/cm² in growth media (GM) containing α-MEM, FBS (10% v/v, ThermoFisher, Cat #16000044), penicillin/streptomycin (10000 U/mL; 1% v/v) and cultured in humidified incubator (37° C., 5% CO₂). The medium was replaced after 3 days and further cultured for 6 days before passage. For passaging, cells were incubated in 0.25% trypsin-EDTA for 2 min at 37° C., detached with a cell scraper, neutralized by using GM, centrifuged, and sub-cultured at a density of 8000 cells/cm². All experiments were performed between 1-2 passage. Osteogenic medium (OM) was prepared by supplementing GM with 10 mM β-glycerophosphate (MilliporeSigma, Cat #G9422), 50 μM ascorbic acid-2-phosphate (MilliporeSigma, Cat #A8960), and 100 nM dexamethasone (MilliporeSigma, Cat #D4902).

### 8. Calcium Imaging

Intracellular cytosolic Ca²⁺ was evaluated by Fura-2 loaded DRG neurons (Khomula E V, et al. (2019) J Neurosci. 39:7061-7073). Fura-2 (ThermoFisher, Cat #F1221) stock aliquots (2 mM in 100% DMSO) were mixed 1:1 in Pluronic F-127 and then 1:500 in Tyrode's solution (140 mM NaCl, 5 mM KCl, 2 mM CaCl₂, 2 mM MgCl₂, 10 mM HEPES, 10 mM glucose, pH 7.4) (Khomula E V, et al. (2019) J Neurosci. 39:7061-7073). DRG samples were washed twice in Tyrode's solution before replacing with Fura-2 loading solution and incubated at room temperature for 40 min. Samples were then washed thrice with Tyrode's solution and incubated for an additional 20 min prior to loading for imaging. Cells were mounted on the translation stage of an Olympus IX81 inverted microscope (Olympus America, Center Valley, PA) and depending on the experimental group, were treated with either adenosine (5 μM), or adenosine (5 μM) along with ADORA1 inhibitor DPCPX (100 nM) for 5 min before stimulating with capsaicin (100 nM). For the experiments involving DPCPX, since DPCPX was dissolved in DMSO a corresponding control culture exposed to the vehicle DMSO was used.

Fura-2 dual excitation and emission was accomplished using 340- and 380-nm excitation filters and a 510 nm emission filter, and cells were visualized with an Olympus UPlan FLN 20×1.3 NA water immersion objective. Light was supplied by a Lambda XL (Sutter Instrument Company, Novato, CA) using variable aperture. Digital images (150-ms exposure) were recorded with a Hamamatsu EM CCD camera (Hamamatsu Photonics, Hamamatsu City, Japan) at 1 s intervals. Imaging was performed by first establishing a baseline intensity ratio (340/380 nm) for the region of interest (ROI) prior to capsaicin (TRPV1 agonist) treatment. The normalized Fura-2 intensity profiles were plotted as the ratiometric intensity divided by the baseline intensity. Peak intensity measurements are reported as the maximum normalized intensity during stimulation. The total number of DRG neurons in a given ROI was determined by adding KCl at the end of the experiment and counting the number of activated DRG neurons, i.e., cells with a ratiometric change.

### 9. Membrane Potential Imaging

DRG neuron potential changes were evaluated with FluoVolt, a voltage sensitive indicator dye (ThermoFisher, Cat #F10488). Fluovolt was loaded according to manufacturer instructions; briefly, loading solution was prepared by adding 10 μL of 10× component B and 1 μL of component A in a 1.5-mL tube, followed by 1 mL of Tyrode's solution. DRG culture medium was removed and washed prior to adding the loading solution, and incubated at room temperature for 30 min. Loading solution was then removed and washed thrice with Tyrode's solution. Imaging was performed on translation stage of an Olympus IX81 inverted microscope (Olympus America, Center Valley, PA, USA, with FITC excitation filter. Digital images (250-ms exposure) were recorded with a Hamamatsu EM CCD camera (Hamamatsu Photonics) at 1 s intervals. Imaging was performed prior to capsaicin stimulation to determine the baseline intensity for the DRG neurons. Fluorescence intensity profiles were reported as the intensity at a given time point divided by the baseline intensity. Peak intensity measurements are reported as the maximum normalized intensity during stimulation.

### 10. RT-qPCR

Cells, DRG, or bone/marrow tissues were analyzed for gene expression by quantitative real-time polymerase chain reaction (RT-qPCR). Nucleic acids were extracted with TRIzol, phase-separated with chloroform, and precipitated in isopropanol. One microgram of RNA was reverse transcribed using iScript cDNA Synthesis Kit (Bio-Rad, Hercules, CA, Cat #1708891) according to the manufacturer's instructions (Hoque J, et al. (2021) Biomaterials. 273:120819). Quantitative PCR was performed with iTaq Universal SYBR green reagent (Bio-Rad, Cat #1725124) with denaturation at 95° C. for 30 sec for one cycle, and amplification (denaturation+annealing/extension) at 95° C. for 5 sec and 60° C. for 30 sec for 40 cycles on a polymerase chain reaction (PCR) cycler (Bio-Rad, CFX96 Touch). The primer sequences used are as follows:

| | Primer Sequence and |
|---|---|
| Primer Description | Sequence Identifier |
| AIR/ADORA1 fwd | CCCCATCGTCTATGCCTTCC |
| | (SEQ ID NO: 01) |
| AIR/ADORA1 rev | CATCGGAAGTGGTCGTTCCA |
| | (SEQ ID NO: 02) |
| 18S ribosomal RNA fwd | ACCAGAGCGAAAGCATTTGCCA |
| | (SEQ ID NO: 03) |
| 18S ribosomal RNA rev | ATCGCCAGTCGGCATCGTTTAT |
| | (SEQ ID NO: 04) |
| NGF fwd | GGGAGCGCATCGAGTTTTG |
| | (SEQ ID NO: 05) |
| NGF rev | TACGCTATGCACCTCACTGC |
| | (SEQ ID NO: 06) |
| TRPV1 fwd | CAGCGAGTTCAAAGACCCAGA |
| | (SEQ ID NO: 07) |
| TRPV1 rev | GCAGAGCAATGGTGTCGTTC |
| | (SEQ ID NO: 08) |
| A2AR/ADORA2A fwd | GCCAGAGCAAGAGGCAGGTAT |
| | (SEQ ID NO: 09) |
| A2AR/ADORA2A rev | TCCCAAAGGCTTTCTCACGG |
| | (SEQ ID NO: 10) |
| A2BR/ADORA2B fwd | ATCTTTAGCCTCTTGGCGGTG |
| | (SEQ ID NO: 11) |
| A2BR/ADORA2B rev | GACCCAGAGGACAGCAATGAT |
| | (SEQ ID NO: 12) |
| A3R/ADORA3 fwd | GCTGTAGACCGATACCTGCG |
| | (SEQ ID NO: 13) |
| A3R/ADORA3 rev | GGAAACTAGCCAGCAAAGGC |
| | (SEQ ID NO: 14) |
| GAPDH fwd | GCACAGTCAAGGCCGAGAAT |
| | (SEQ ID NO:15) |
| GAPDH rev | GCCTTCTCCATGGTGGTGAA |
| | (SEQ ID NO: 16) |
| RUNX2 forward | TGGCCGGGAATGATGAGAAC |
| | (SEQ ID NO: 17) |
| RUNX2 reverse | TGAAACTCTTGCCTCGTCCG |
| | (SEQ ID NO: 18) |
| SP7 forward | TGCCTGACTCCTTGGGACC |
| | (SEQ ID NO: 19) |
| SP7 reverse | TAGTGAGCTTCTTCCTCAAGCA |
| | (SEQ ID NO: 20) |
| IBSP forward | TCCACACTTTCCACACTCTCG |
| | (SEQ ID NO: 21) |
| IBSP reverse | CTTTCTGCATCTCCAGCCTTC |
| | (SEQ ID NO: 22) |
| BGLAP forward | GCTACCTTGGAGCCTCAGTC |
| | (SEQ ID NO: 23) |
| BGLAP reverse | AGGGTTAAGCTCACACTGCTC |
| | (SEQ ID NO: 24) |

The expression level of each target gene was normalized to the housekeeping gene and to their respective controls and presented as fold change expressed as 2^{-ΔΔCt} values. NGF expression was normalized to Gapdh, and other gene expressions were normalized to 18S rRNA.

### 11. Immunofluorescence Staining

In vitro cultured DRG neurons, frozen DRG sections, and paraffin-embedded tibiae sections were stained and imaged.

a. In Vitro Cultured DRG Neurons

Cells were fixed in 4% PFA for 15 min, permeabilized in 0.1% Triton X-100 in PBS for 10 min, followed by blocking for 1 hr in 3% BSA in PBS at room temperature. Cells were co-stained with TUBB3 (Novus Biologicals, Littleton, CO, Cat #NB100-1612, 1:500 dilution in 3% BSA) and TRPV1 (Novus Biologicals, Cat #NBP1-71774, 1:300 dilution in 3% BSA) antibodies at 4° C. overnight. Cells were washed with PBS at room temperature for 10 min three times and incubated with secondary antibodies donkey anti-chicken AlexaFluor 488 (Jackson ImmunoResearch, West Grove, PA, Cat #703-545-155, 1:300 dilution) and donkey anti-rabbit AlexaFluor 647 (Jackson ImmunoResearch, Cat #711-605-152, 1:300) for 1 hr at room temperature.

b. Frozen DRG Sections

Sections were heated for antigen retrieval in citrate buffer (MilliporeSigma, Cat #C9999) for 20 min, permeabilized in 0.1% Triton X-100 in PBS for 10 min, followed by blocking for 1 hour in blocking solution comprised of 3% BSA, 0.26 M glycine, 5% normal donkey serum in Tris buffered saline (TBS) at room temperature. Sections were stained with TRPV1 (Novus Biologicals, Cat #NBP1-71774, 1:300 dilution in blocking solution) antibodies, or ADORA1 (Proteintech, Rosemont, IL, Cat #55026-1-AP, 1:100 dilution in blocking solution) antibodies at 4° C. overnight. Sections were washed with TBS with 0.1% Tween-20 (TBS-T) at room temperature for 10 min thrice, and then incubated with secondary antibodies donkey anti-rabbit AlexaFluor 647 (Jackson ImmunoResearch, Cat #711-605-152, 1:300 dilution in blocking solution) for 1 hr at room temperature. Nissl stain (ThermoFisher, Cat #N21482, 1:100 dilution in PBS) was used to tain for DRG neuron at room temperature for 20 min.

c. Paraffin-Embedded Tibiae Sections

Sections were heated for antigen retrieval in citrate buffer (MilliporeSigma, Cat #C9999) for 20 min, permeabilized in 0.1% Triton X-100 in PBS for 10 min, followed by blocking for 1 hr in blocking solution comprised of 3% BSA, 0.26 M glycine, 5% normal donkey serum in Tris buffered saline (TBS) at room temperature. Sections were co-stained with Td-tomato (MyBiosource, San Diego, CA, Cat #MBS448092 1:100 dilution in blocking solution) and ADORA2B (MyBiosource, Cat #MBS8207549, 1:100 dilution in blocking solution) antibodies at 4° C. overnight. Sections were washed with TBS-T at room temperature for 10 min thrice, and then incubated with secondary antibodies donkey anti-goat FITC (Jackson ImmunoResearch, Cat #705-096-147) and donkey anti-rabbit AlexaFluor 647 (Jackson ImmunoResearch, Cat #711-605-152, 1:300) for 1 hr at room temperature. Finally, all samples were washed with PBS or TBST, covered with mounting solution (ThermoFisher, Cat #P36971), sealed and imaged using TRITC, Cy5, GFP and DAPI filters on a Keyence BZ-X700 microscope. Fluorescence intensity was quantified by ImageJ software and presented in arbitrary units.

### 12. Safranin O Staining

To visualize the callus remodeling and bone tissue formation, tissue sections were stained with 1% safranin-O (Millipore Sigma, Cat #S8884) for 1 hr and counter-stained with 0.02% Fast Green (Millipore Sigma, Cat #F7258) for 1 min. The sections were then dehydrated in an ethanol gradient, mounted with Cytoseal (ThermoScientific, Cat #23-244256), and imaged by using a Keyence BZ-X710 microscope.

### 13. Measurement of Adenosine in Plasma

Peripheral blood was collected and immediately incubated in ice cold stop solution at a 1:2 ratio (blood to stop solution) to inhibit degradation of adenosine (Feldman M D, et al. (1992) Clin Chem. 38:256-262). Stop solution comprised of 0.2 mM dipyridamole (Tocris, Minneapolis, MN), 5 μM erythro-9 (2-hydroxy-3-nonyl)-adenine (EHNA; Tocris), 62 μM Adenosine 5′-(α,β-methylene) diphosphate sodium salt (APCP), 5 mM EDTA, and 25 IU/mL heparin (Tocris) in PBS. The blood was centrifuged at 2000 rcf for 10 min at 4° C. to separate cells from plasma. Adenosine assay kit (Cell Biolabs, San Diego, CA, Cat #MET-5090) was used to measure adenosine levels from plasma according to manufacturer's protocol. Briefly, a reaction mixture comprised of fluorometric Probe, HRP, adenosine deaminase, purine nucleoside phosphorylase, xanthine oxidase, assay buffer and a control mix comprised of fluorometric Probe, HRP, purine nucleoside phosphorylase, xanthine oxidase, assay buffer were made and mixed with 50 μL of plasma for 15 min. at room temp. The relative fluorescence unit (RFU) was measured using a microplate reader with excitation at 570 nm and emission at 590 nm. Adenosine concentration was determined using a standard curve of known concentrations and subtracted for the background by using the values in control mix.

### 14. Statistical Analyses

Statistical analyses were carried out using GraphPad Prism 9 or SPSS v.20. Mann-Whitney U test was used to compare between two groups. Statistical analysis comparing multiple groups was performed by Kruskal-Wallis with Dunn's post hoc test or Friedman test with Wilcoxon signed-rank test, as appropriate. For all comparisons, two-tailed tests were used and p<0.05 was considered statistically significant. Cohen's d or r were used to calculate standardized effect size using SPSS v.20, as appropriate. Power analysis was performed using G*Power.

### Example 1

### Peripheral Neurons Innervating Fractured Bone Exhibited Enhanced Nociceptive Phenotype

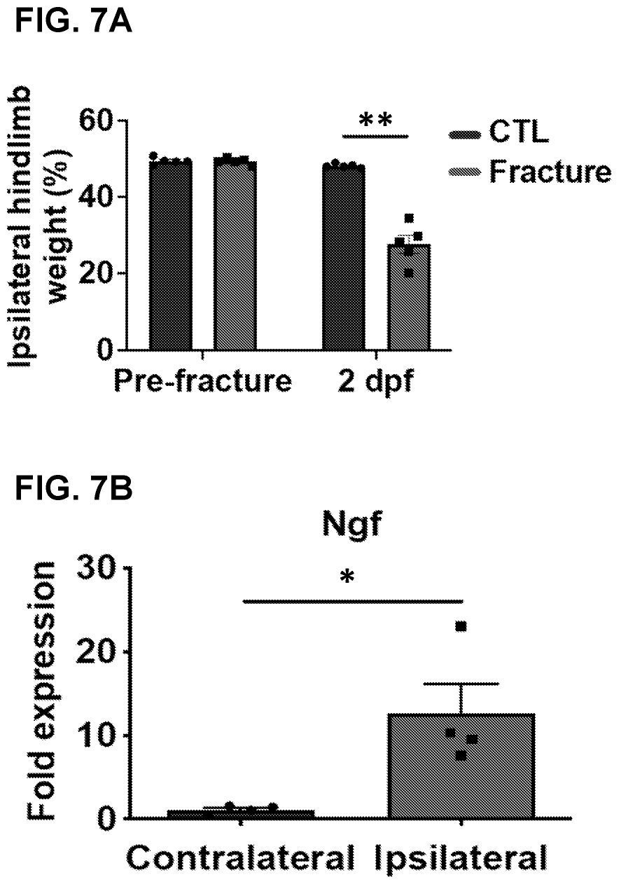

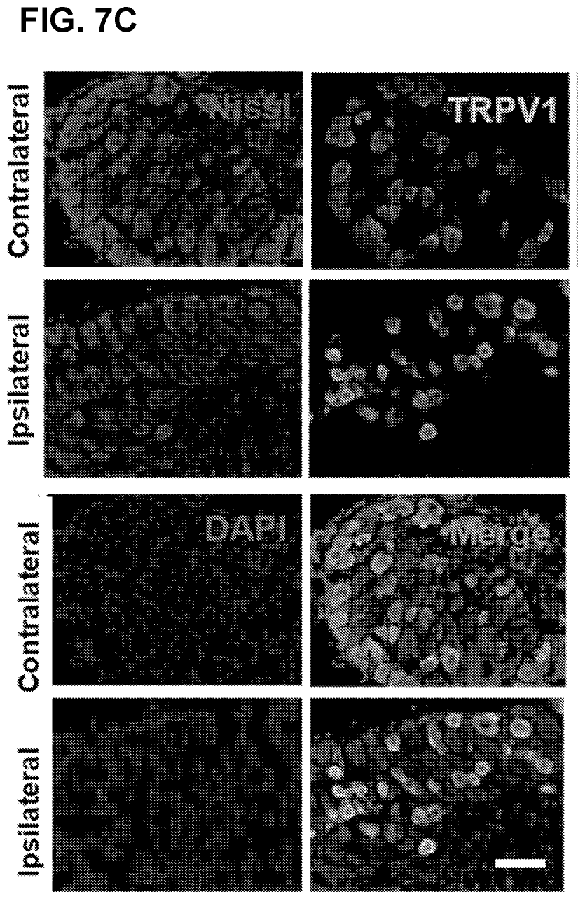

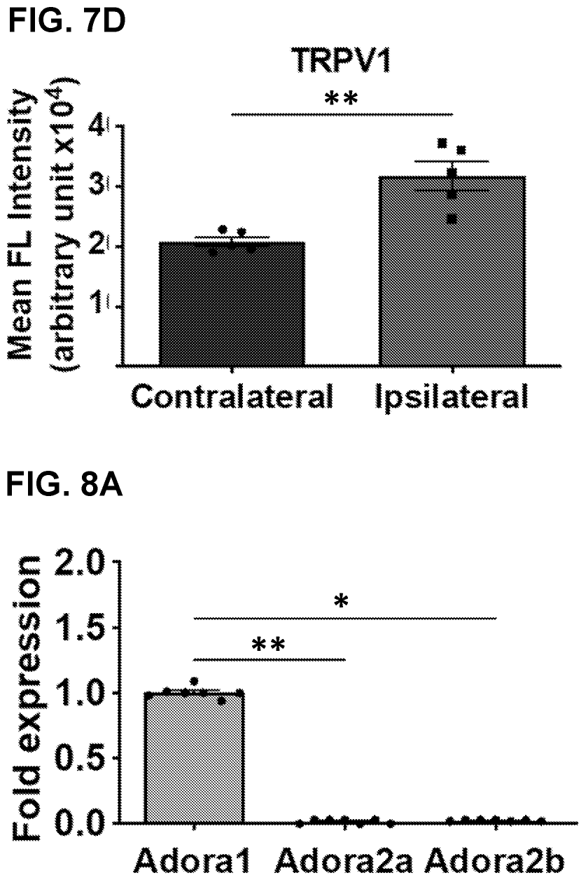

Injury to bone has been shown to elicit spontaneous and palpation-induced nocifensive behaviors, reduced weight bearing, decreased rearing, and increased mechanical, thermal, and cold allodynia in mice (Majuta L A, et al. (2015) Pain. 156:157-165; Zhang L, et al. (2018) Front Pharmacol. 9:412; Minville V, et al. (2008) Anesthesiology. 108:467-472). Fractures produce an array of inflammatory cytokines, chemokines, neurotrophins, and neurokines that have been shown to contribute to inflammatory pain (Brazill J M, et al. (2019) J Bone Miner Res. 34:1393-1406; Sun S, et al. (2020) Bone. 131:115109; Loi F, et al. (2016) Bone. 86:119-130). One such molecule is nerve growth factor (NGF), which is upregulated following fracture and its inhibition has been shown to attenuate fracture pain (Li Z, et al. (2019) J Clin Invest. 129:5137-5150; Majuta L A, et al. (2015) Pain. 156:157-165). The nociceptive effect of NGF is partially due to its role in promoting the translocation of transient receptor potential cation channel subfamily V member 1 (TRPV1) to the cell surface membrane and upregulation of TRPV1 expression (Zhang X, et al. (2005) EMBO J. 24:4211-4223; Xue Q, et al. (2007) J Neurochem. 101:212-222). Studies have found TRPV1-expressing neurons in L3 and L4 DRG are pivotal to nociception associated with bone trauma and bone cancer of rodent hindlimbs (Morgan M, et al. (2019) Bone. 123:168-175; Ghilardi J R, et al. (2005) J Neurosci. 25:3126-3131; Kawarai Y, et al. (2014) Yonsei Med. J. 55:185-190). As weight bearing has been used in animal models to assess fracture pain (Magnusdottir R, et al. (2021) Osteoporos Int. 32:2347-2359), a mouse model of stabilized unilateral tibial fracture was used to assess post-fracture pain. Using an incapacitance meter, the data showed the static weight bearing of ipsilateral (fractured) hindlimb was decreased at 2 days post-fracture (dpf; FIG. 7A). The decreased weight bearing was associated with an upregulation of Ngf expression (FIG. 7B), and higher levels of TRPV1 expression in ipsilateral Nissl-positive DRG neurons compared to contralateral DRG neurons by immunofluorescence imaging at 3 dpf (FIG. 7C and FIG. 7D). These results indicated that the DRG neurons innervating the fractured limbs exhibited elevated nociception, which were associated with increased Ngf and TRPV1 expression.

### Example 2

### Adenosine Attenuated NGF-Induced Sensitization of DRG Neurons Through Adenosine A1 Receptor

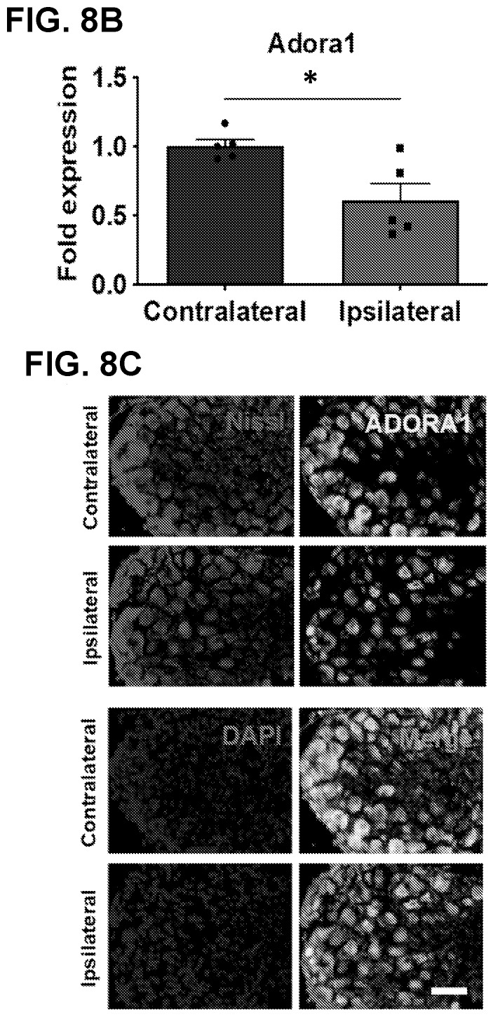

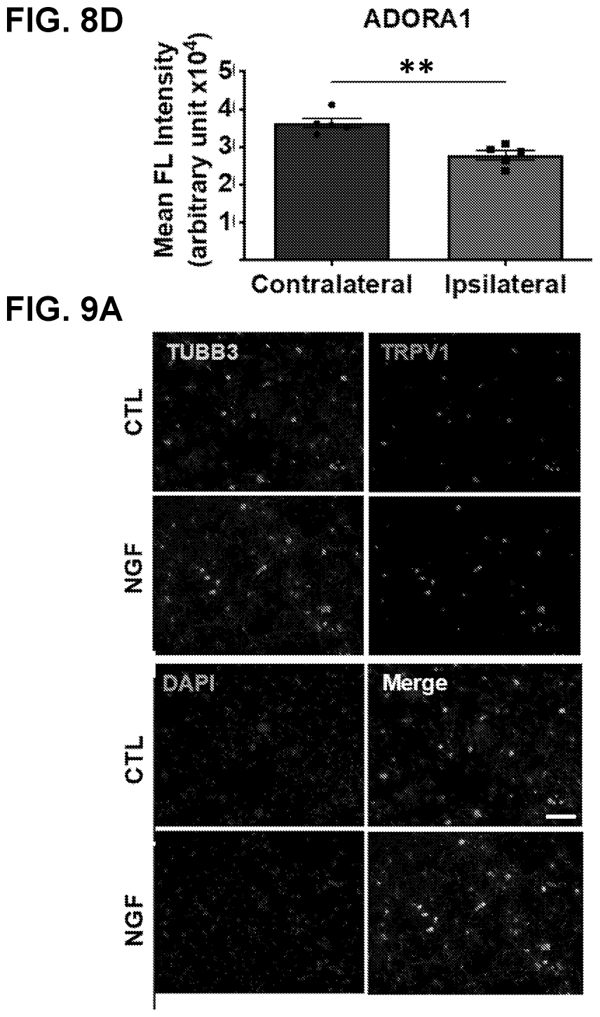

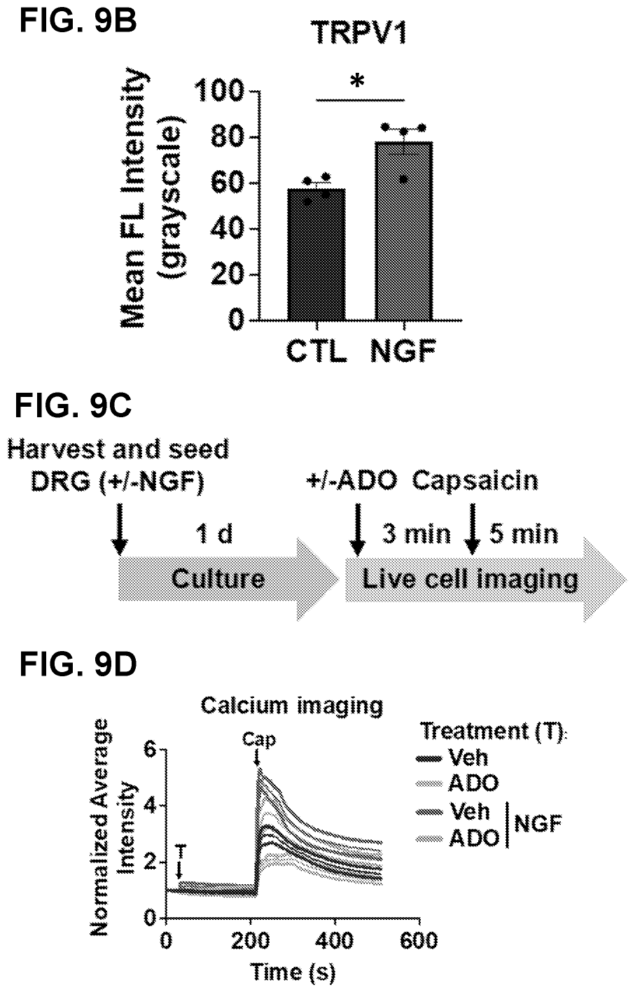

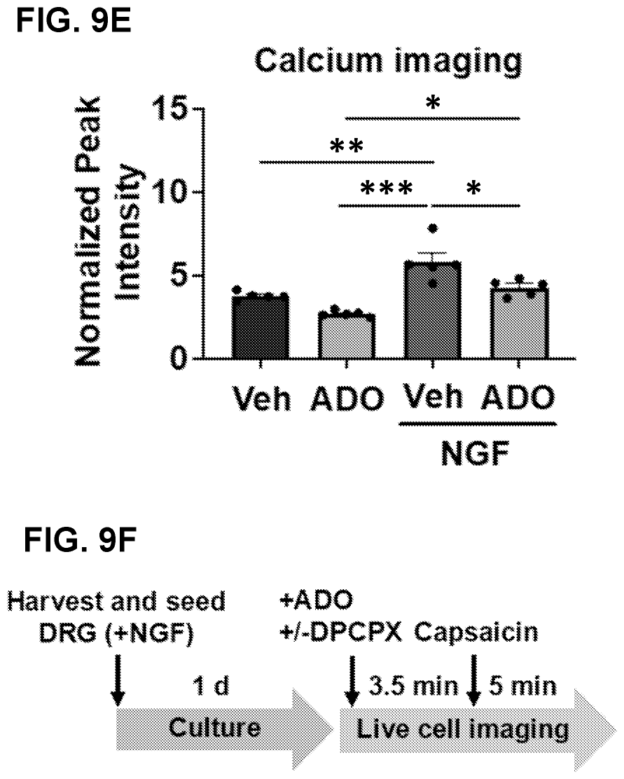

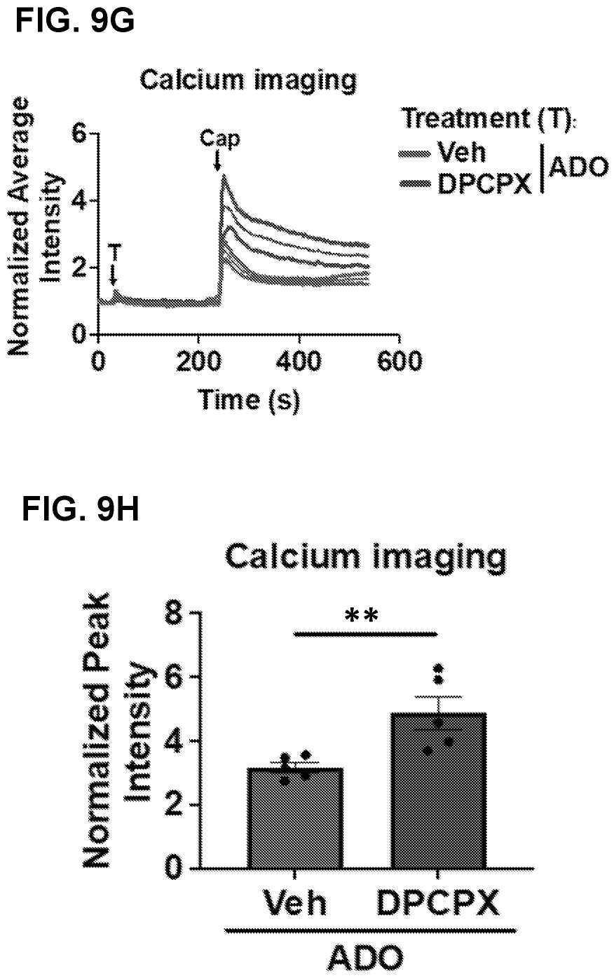

To determine the relative expression of adenosine subtypes in DRG neurons, a gene expression analyses was performing using whole DRG from healthy mice. The data showed that Adora1 was expressed at a significantly higher level than Adora2a and Adora2b, while Adora3 was undetected (FIG. 8A). Gene expression of DRG from fractured animals showed lower expression of Adora1 in ipsilateral DRG neurons compared to contralateral DRG neurons (FIG. 8B). Immunofluorescence staining of ipsilateral and contralateral DRG corroborated the gene expression (FIG. 8C and FIG. 8D). As Ngf expression was elevated in the fractured tibiae, NGF was used as a model molecule for in vitro sensitization of DRG neurons harvested from unfractured mice and examined the effect of adenosine to attenuate the increased nociceptive phenotypic and functional changes induced by NGF. The data demonstrated that 1 day incubation with NGF significantly increased TRPV1 receptor expression in tubulin beta 3 class III (TUBB3)-positive neurons, a marker for neurons (FIG. 9A and FIG. 9B). Subsequently, calcium imaging was performed on dissociated DRG neurons in the presence or absence of NGF, followed by incubation with or without adenosine (FIG. 9C).

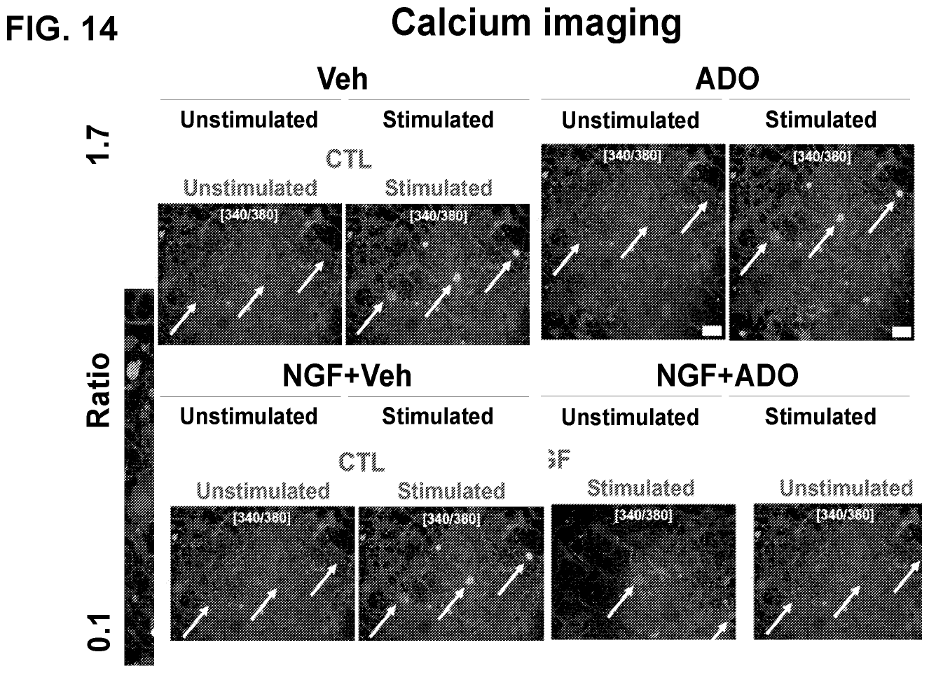

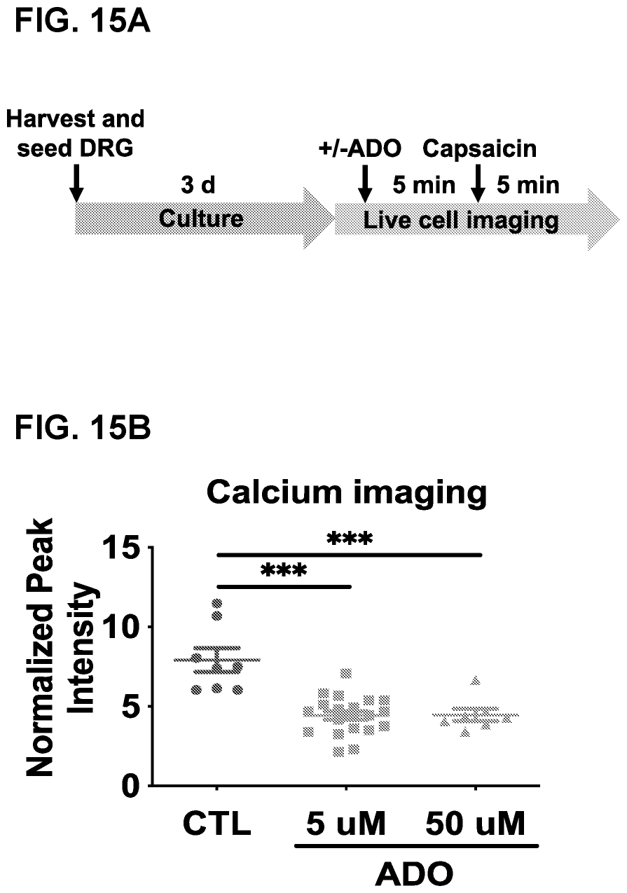

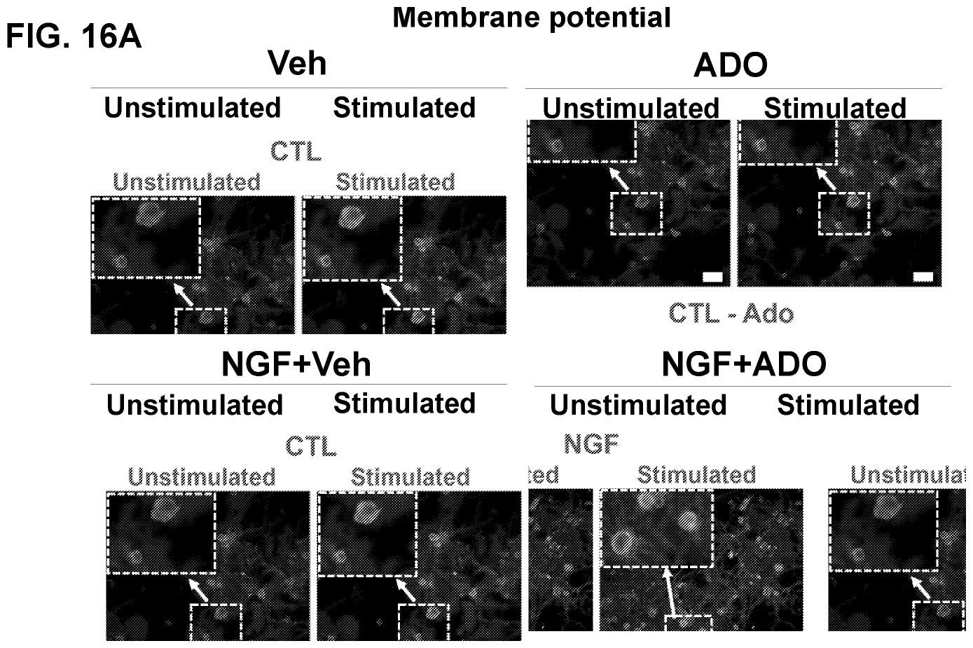

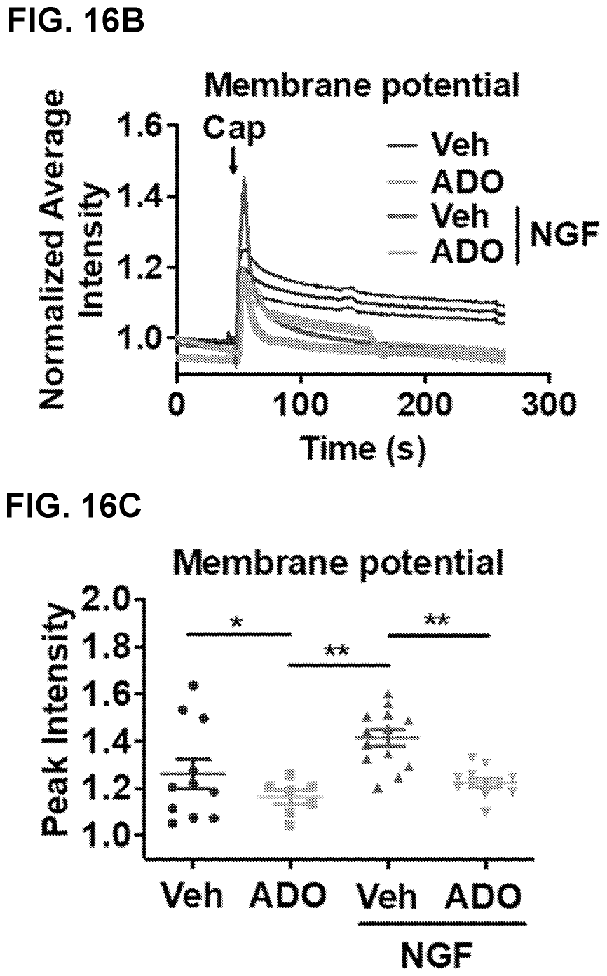

The TRPV1 agonist capsaicin is commonly used to assess the functional responsiveness of nociceptive neurons in vitro (Hanack C, et al. (2015) Cell. 160:759-770) and DRG neurons in all subsequent imaging experiments were also stimulated with capsaicin. Results showed that the exposure of DRG neurons to NGF increased their functional responses to capsaicin, which was attenuated by adenosine (FIG. 14, FIG. 9D and FIG. 9E). The adenosine concentration used was identified from an initial screening (FIG. 15A-FIG. 15B). To determine whether ADORA1 was responsible for adenosine-mediated decrease in functional responses, calcium imaging was performed on dissociated DRG exposed to NGF and treated with adenosine in the presence or absence of ADORA1 inhibitor 8-Cyclopentyl-1,3-dipropylxanthine (DPCPX) (FIG. 9F). Results showed an increase in the average and peak fluorescence intensity upon treatment with DPCPX compared to the vehicle control lacking DPCPX (FIG. 9G and FIG. 9H). The effects of adenosine on neuronal sensitivity were confirmed by measuring the membrane potential of dissociated DRG neurons in vitro using Fluo Volt, a voltage-sensitive dye (FIG. 16A-FIG. 16C). FluoVolt imaging showed that the NGF exposure increased the membrane potential, but the increase was attenuated in the presence of adenosine (FIG. 16A-FIG. 16C).

### Example 3

### Local Delivery of Adenosine Improved Weight Bearing

The in vitro data demonstrated the ability of adenosine to inhibit functional responses of DRG neurons including those sensitized by the fracture-associated pro-nociceptive neurokine NGF. The pain-relieving effect of adenosine was next examined in animals with fracture trauma by local delivery of adenosine following fracture. To aid local delivery, poly(ethylene glycol)-co-6 aminocaproic acid macroporous hydrogels functionalized with 3-(acrylamido)phenylboronic acid (3APBA) was used, where the PBA moieties were used to load the adenosine molecules (Zeng Y, et al. (2020) Adv Mater. 32:e1906022). Details about the fabrication of the macroporous hydrogels are described supra. Successful synthesis of the hydrogel precursors, poly(ethylene glycol) diacrylate (PEGDA) and N-acryloyl-6-aminocaproic acid (A6ACA) from poly(ethylene glycol) (PEG) and 6-aminocaproic acid (6ACA) respectively, was assessed by FTIR (FIG. 1-FIG. 2) and NMR (FIG. 3-FIG. 4). The fabricated macroporous hydrogel (PEG-6ACA-PBA) was characterized by Fourier transform infrared (FTIR) spectroscopy (FIG. 5) and proton nuclear magnetic resonance (1HNMR) (FIG. 6) spectroscopy. Adenosine loading was determined by UV/Vis spectrophotometer at a wavelength of 260 nm, which revealed that ˜0.26±0.04 mg adenosine was incorporated per 1 mg of the macroporous hydrogel. The in vitro release kinetics of adenosine from the macroporous hydrogels showed an initial rapid release followed by a sustained release (FIG. 17). Following fracture surgery, the animals were implanted with the macroporous hydrogels with or without adenosine.

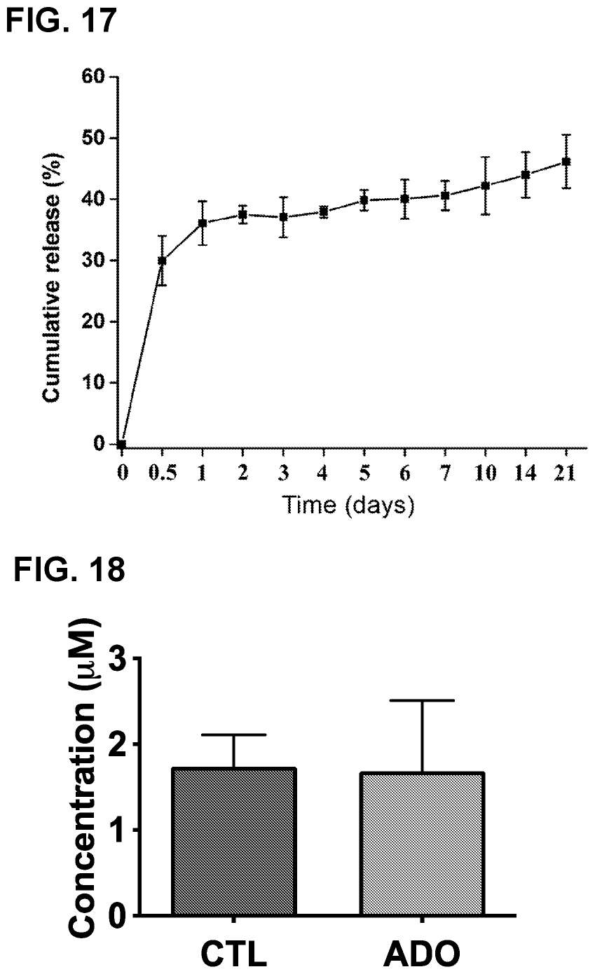

Static weight bearing of mice was measured to examine the effect of adenosine on mitigating fracture pain. The experimental timeline is described in FIG. 10A. Fracture surgery was performed in the absence of analgesia and macroporous hydrogels measuring approximately 4 mm (length)×2 mm (width)×1 mm (thick) containing either 1.05±0.1 mg adenosine (ADO) or no adenosine (CTL) was immediately implanted next to the fracture site. Ipsilateral hindlimb weight bearing of adenosine and control-treated mice were measured using an incapacitance meter and expressed as a percent weight bearing of both hindlimbs. Tibial fracture resulted in a significant reduction in ipsilateral weight bearing percentage in the control group at 2 dpf, 4 dpf, and 8 dpf compared to pre-fracture (baseline) (FIG. 10B). The ipsilateral weight bearing of adenosine-treated limbs was significantly decreased at 2 days post-fracture and returned back to the baseline by 8 days. When comparing the weight bearing of adenosine-treated limbs to that of the untreated control limbs, the weight bearing of adenosine-treated limbs was significantly higher compared to control-treated limbs at all post-fracture timepoints. Furthermore, the positive impact of adenosine treatment on weight bearing was associated with a large effect size at each timepoint (FIG. 10C). The percent change in weight bearing from baseline was also calculated (FIG. 10D), which showed significant decreases in weight bearing from the baseline at 2 dpf and 4 dpf for both the treated and untreated groups; however, the degree of decrease in weight bearing was less for the adenosine-treated group. By 8 dpf, the weight bearing ability of animals treated with adenosine had returned to baseline while those of the control groups still exhibit significantly reduced weight bearing ability (FIG. 10D). To determine whether the locally delivered adenosine resulted in increased adenosine level in the circulation, the concentration of adenosine was measured in the peripheral blood 3 days after implantation of biomaterials loaded with adenosine following tibial fracture. No significant increase in the adenosine level when compared to control biomaterials was identified. (FIG. 18).

### Example 4

### Local Delivery of Adenosine Improved Open Field Movement

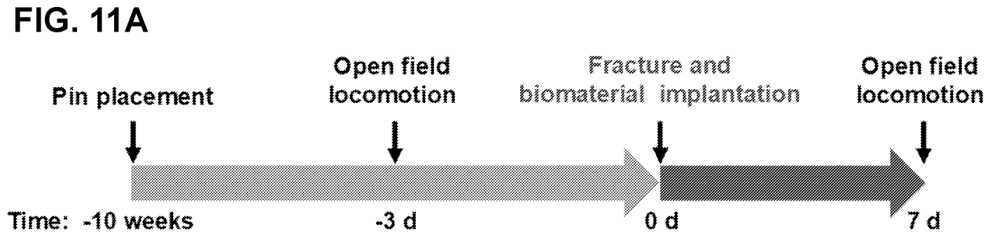

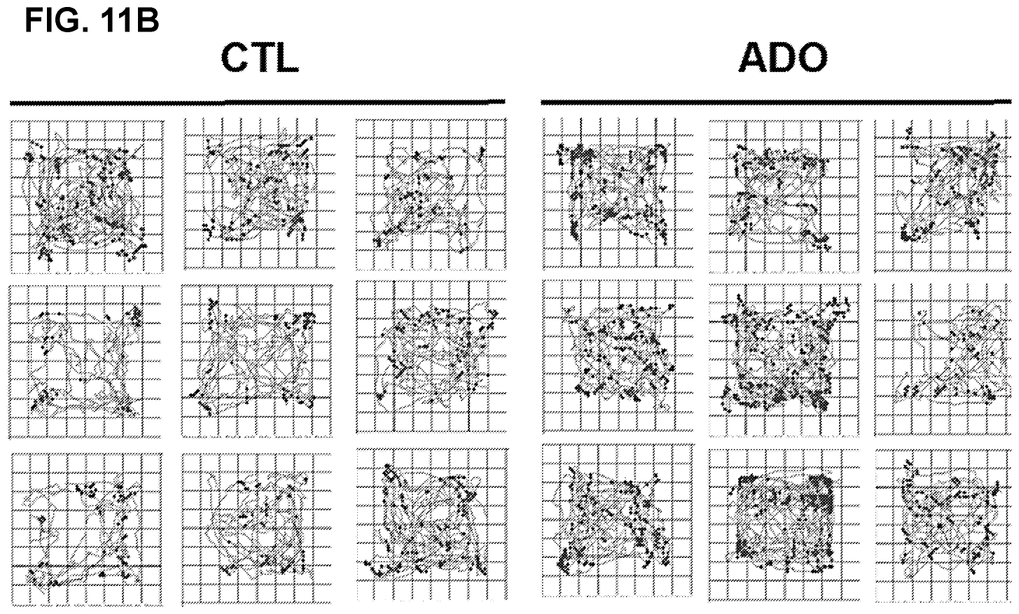

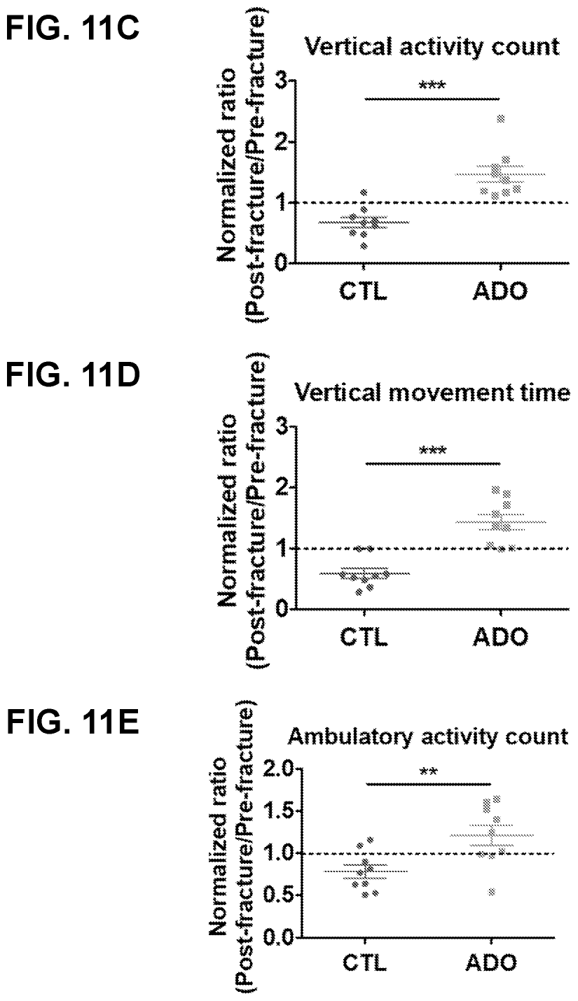

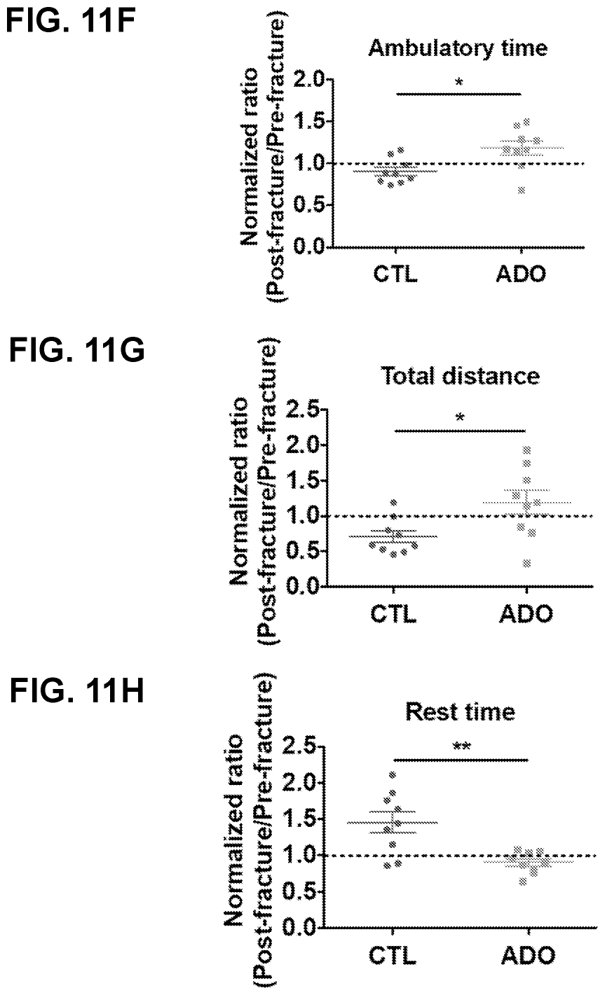

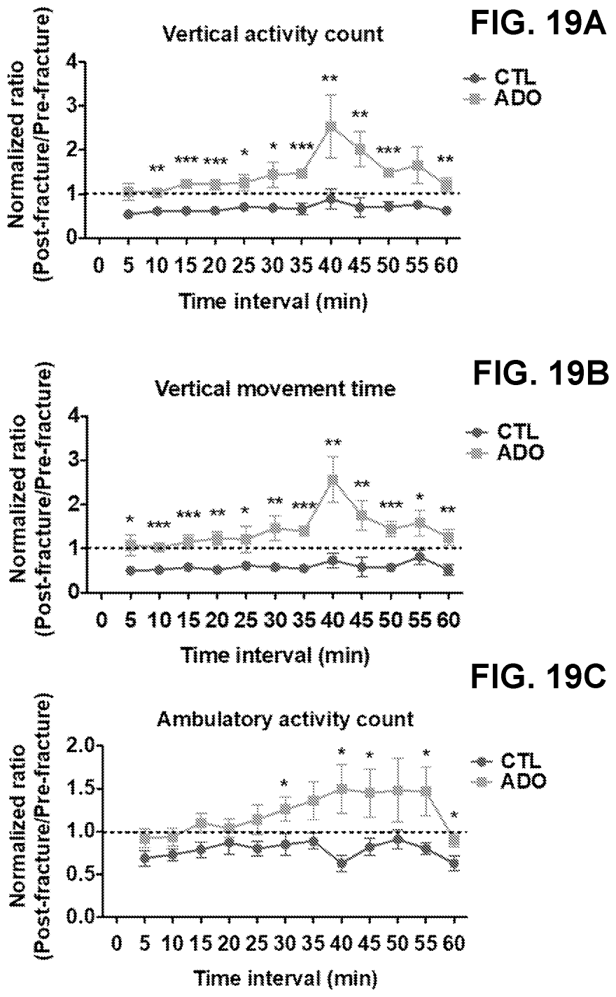

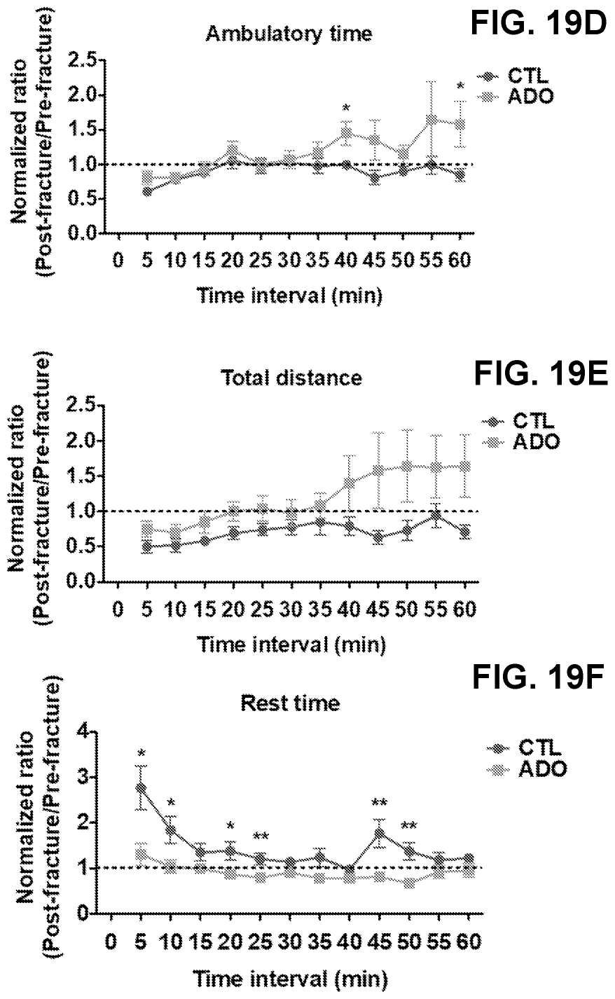

As vertical and ambulatory activity of open field movement has been used to assess pain and functional outcomes (Majuta L A, et al. (2018) Pain. 159:2285-2295), animals were subjected to an enclosed open field and voluntary activities were recorded for a duration of 60 min. The timeline of surgery and open field activity is shown in the schematic of the experimental design (FIG. 11A). Fracture surgery was performed in the absence of analgesia and macroporous containing ADO or no ADO (CTL) was immediately implanted next to the fracture site. The open field test was performed at 7 days post-fracture and implantation and representative plots of activities for each animal (5 min duration between 30th and 35th min) were depicted by red dots representing the vertical activity or rearing, and blue lines representing the horizontal activity or ambulation (FIG. 11B). The activities for the whole duration were collected as 5 min intervals and represented as a ratio of post-fracture to pre-fracture (FIG. 19A-FIG. 19F). Adenosine-treated animals had higher levels of activity than the control group animals for most of the 5-min intervals including vertical activity count, vertical movement time, ambulatory activity count, ambulatory time, total distance, and lower rest time (FIG. 19A-FIG. 19F). The sum of activities over the whole duration also showed significantly higher vertical activity counts, vertical movement time, ambulatory activity counts, ambulatory time, total distance, and lower rest time for the cohorts treated with adenosine compared to the control group (FIG. 11C-FIG. 11H). The effect of adenosine treatment was associated with large effect sizes across all of the measured parameters (FIG. 11I).

### Example 5

### Local Delivery of Adenosine Promoted Fracture Healing

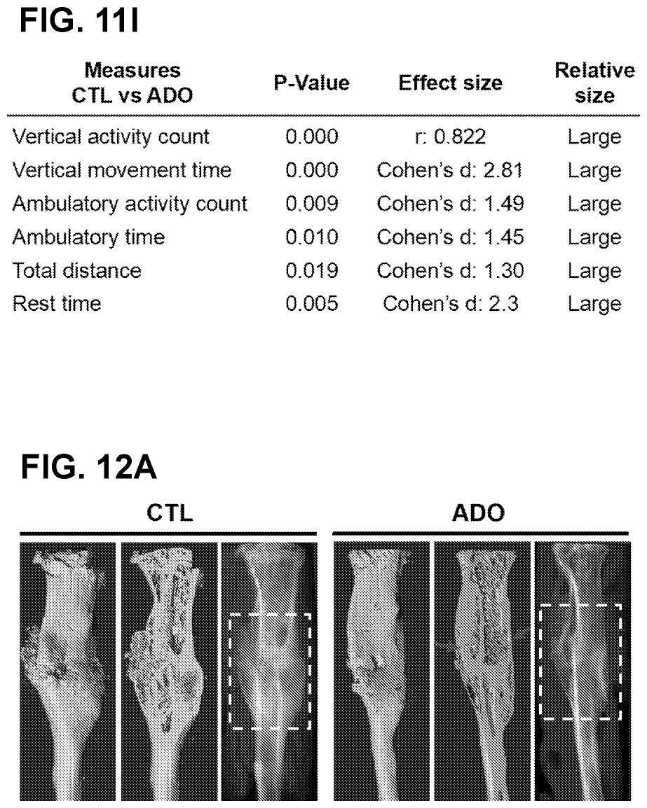

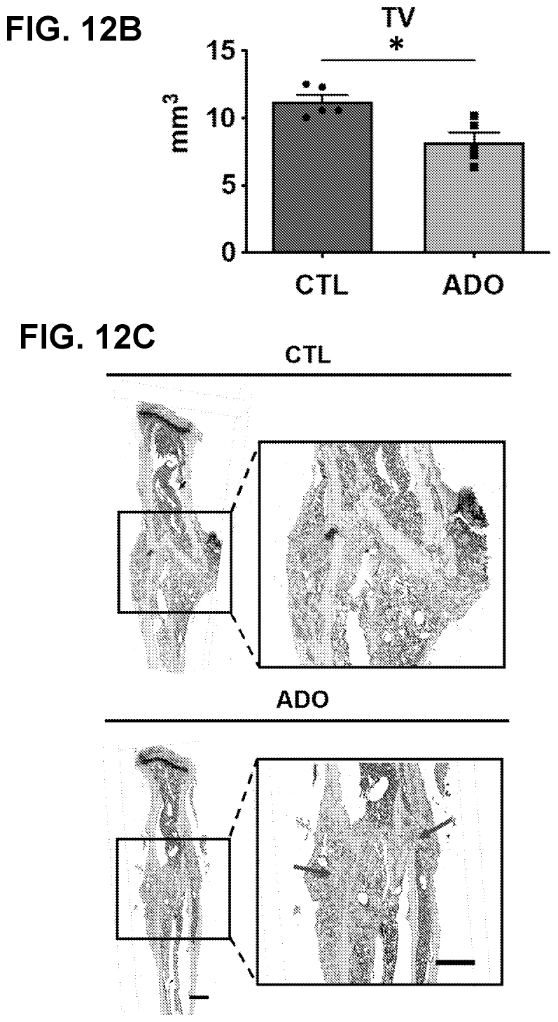

Microcomputed tomography (microCT) images were used to assess fracture healing at 21 days post-fracture. Results showed local delivery of adenosine accelerated fracture healing as demonstrated by 3D reconstructed microCT images (intact and cut views) and corresponding radiographs of the injured tibiae with treated groups demonstrate improved bridging of cortical bone with less trabecular bone (red arrows), and smaller calluses (highlighted white dash box), akin to that seen in Zeng Y, et al. (2020) Adv Mater. 32:e1906022. (FIG. 12A). Analyses of microCT images indicated lower total volume in cohorts treated with adenosine (FIG. 12B). Similar to microCT analyses, histological images showed more cortical bone across the fracture site (red arrows) and smaller callus size in adenosine-treated groups compared to controls at 21 days post-fracture (FIG. 12C). Smaller total volume and callus size has been shown to indicate accelerated healing (O'Neill K R, et al. (2012) Bone. 50:1357-1367).

### Example 6

### Adenosine A2B Receptor Was Highly Expressed in Osteoprogenitors of Regenerating Bone

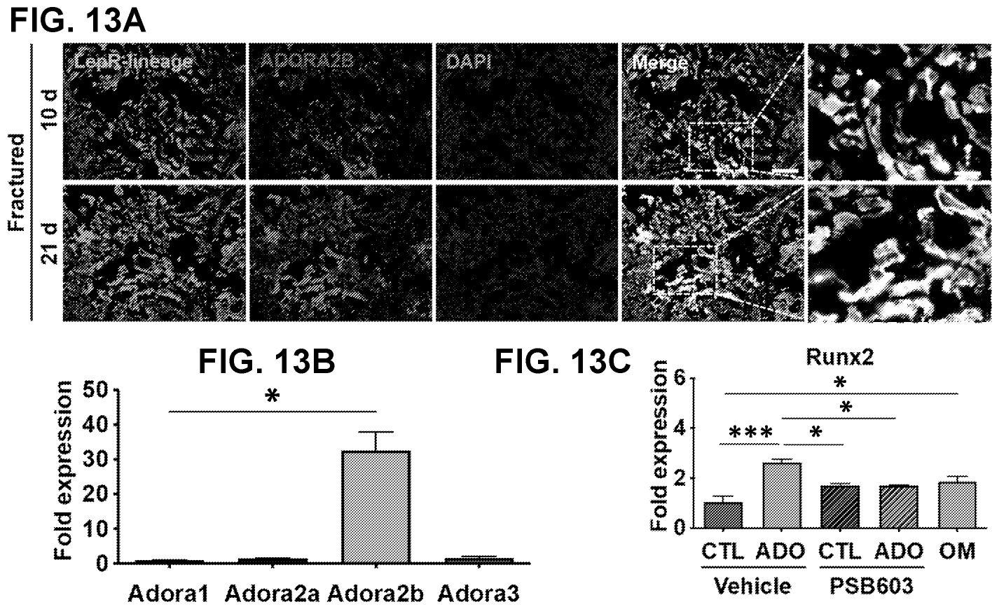

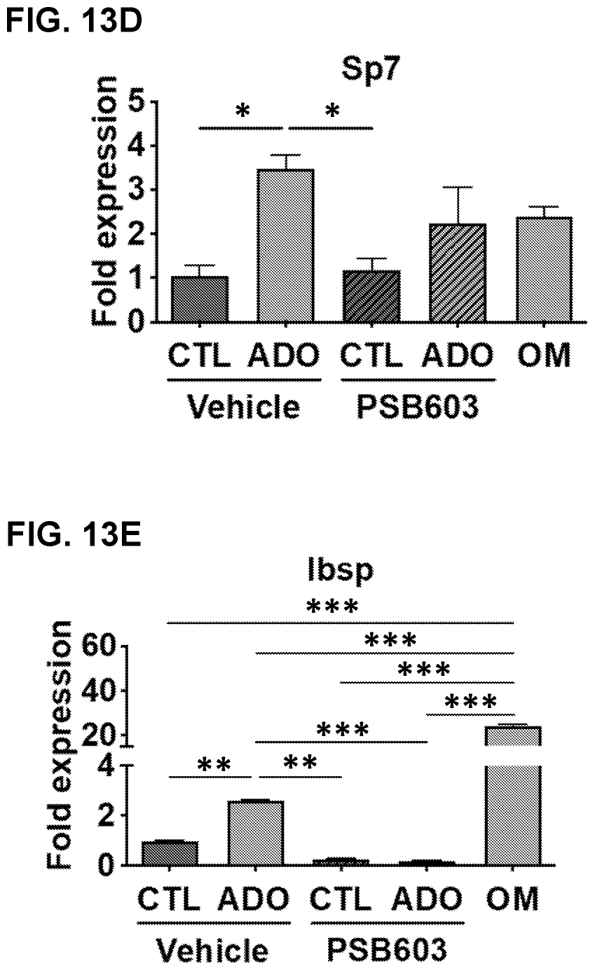

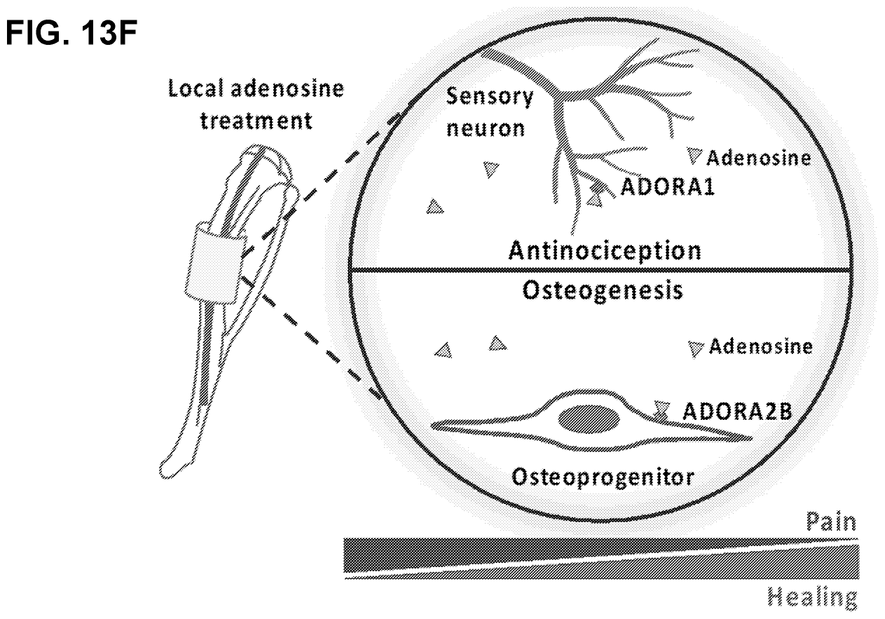

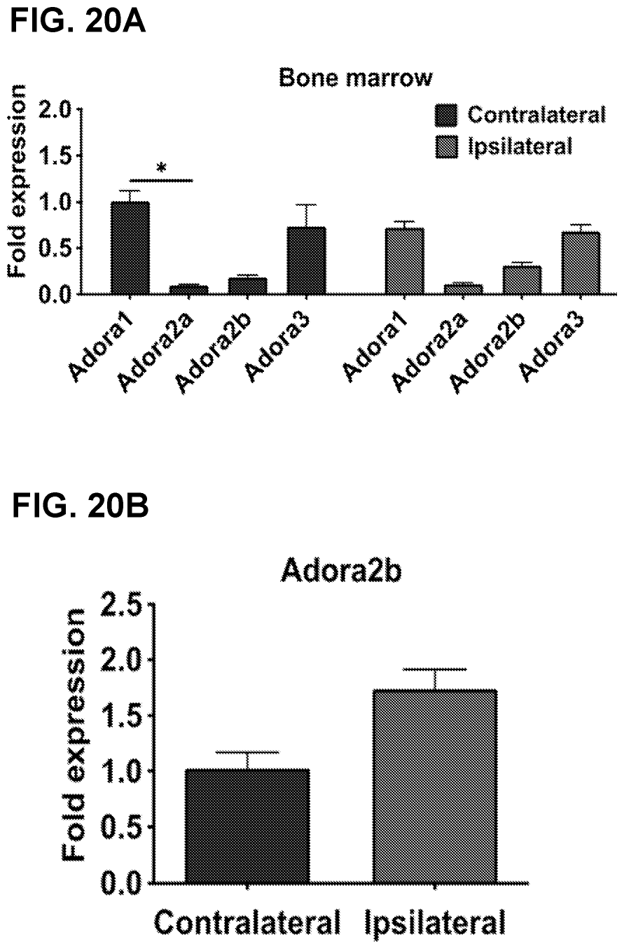

Given the key role played by ADORA2B in osteogenic differentiation of stem cells (Carroll S H, et al. (2012) J Biol Chem. 287:15718-15727; Shih Y R, et al. (2014) Proc Natl Acad Sci USA. 111:990-995; Rao V, et al. (2015) Front Bioeng Biotechnol. 3:185; Kang H, et al. (2015) Biomacromolecules. 16:1050-1061; Kang H, et al. (2016) Sci Adv. 2:e1600691) and fracture healing (Carroll S H, et al. (2012) J Biol Chem. 287:15718-15727), the expression of ADORA2B in progenitor cells in the fracture callus was examined. The leptin receptor (LepR) has been demonstrated to be a marker of bone marrow progenitor cells (Zhou B O, et al. (2014) Cell Stem Cell. 15:154-168; Jeffery E C, et al. (2022) Cell Stem Cell. 29:1547-1561). These cells proliferate after injury and represent a cell source for bone tissue formation following injury (Zhou B O, et al. (2014) Cell Stem Cell. 15:154-168). Using Lepr-cre; tdTomato conditional reporter mice to trace LepR (+) lineage cells, the expression of ADORA2B in LepR lineage progenitors after fracture was examined by immunofluorescent staining for ADORA2B. The data showed abundant co-localization of ADORA2B expression in LepR lineages in the fracture calluses at 10 days post-fracture and 21 days post-fracture (FIG. 13A). Furthermore, gene expression analyses of adenosine receptors in bone marrow-derived MSCs demonstrated significantly higher levels of Adora2b expression compared to other adenosine receptor subtypes (FIG. 13B). The role of ADORA2B activation on osteogenic differentiation of MSCs was also examined. Results showed that adenosine upregulated gene expression of osteoblast transcription factors Runx2 and Sp7, and osteoblast markers Ibsp in the absence of osteogenic induction medium (FIG. 13C-FIG. 13E). This adenosine mediated upregulation of osteogenic markers was attenuated in the presence of ADORA2B-specific inhibitor PSB 603 (FIG. 13C-FIG. 13E). The expression levels of different adenosine receptors was also examined in the bone marrow and bone tissues of contralateral and ipsilateral tibiae at 5 days post-fracture. Data showed expression of all 4 adenosine receptors in contralateral and ipsilateral bone and bone marrow tissues and expression levels ranked from highest to lowest were: Adora1>Adora3>Adora2b>Adora2a (FIG. 20A). The expression level of Adora2b in the bone/marrow slightly increased during regeneration (FIG. 20B).

Fractures and associated pain (including post-surgical pain) are common clinical problems. Currently, NSAIDs and opioid analgesics are used to treat fracture pain; while these approaches are effective in managing pain, they can be associated with various side effects including interference with healing. Considering the limitations associated with NSAIDs and opiates, development of novel therapies to attenuate fracture pain without interfering with the healing is highly desirable. In this study, the potential of local delivery of adenosine to attenuate fracture pain while promoting fracture healing with improved limb function as studied. These data show that adenosine can simultaneously elicit osteoanabolic and pain-mitigating functions. Specifically, adenosine activates ADORA1 on sensory neurons and inhibit neuronal excitation to mitigate pain, while stimulating ADORA2B on osteoprogenitors to promote osteogenesis and fracture healing (FIG. 13F). Although pharmacological activation of individual adenosine receptor subtypes using synthetic agonists is a viable strategy to either attenuate fracture pain or promote healing, adenosine was used due to its broader beneficial effects and ability to target multiple adenosine receptor subtypes and different cell populations.

### Summary of Results for Examples 1-6

Here, a biomaterial-assisted local and sustained delivery of adenosine was leveraged to regulate adenosine signaling over a prolonged period of time. The in vivo studies demonstrated that biomaterial-assisted local delivery of adenosine attenuated fracture/surgical pain in the absence of other analgesics. Specifically, weight bearing of the injured limb treated with adenosine was significantly higher with large effect sizes indicating less pain. Similarly, open field activity of animals treated with adenosine was also significantly improved and demonstrated a large effect size indicating adenosine not only reduced pain but also improved limb function. Pain diminished after the bone healed, yet the pain relief by adenosine treatment was due to its analgesic effect and not due to its effect on bone healing as the pain responses was assessed at early timepoints. Together the open field activity and weight bearing measurements at 7 days post-injury and 8 days post-injury indicated sustained pain reduction and improved limb function in treated animals.

In addition to anti-nociception, the ubiquitous expression of multiple adenosine receptor subtypes present in various cell types within the bone tissue offered other potential benefits such as the ability to promote bone regeneration. In this study, the co-localization of ADORA2B was demonstrated in LepR osteoprogenitors in vivo, confirming that the role of ADORA2B in osteogenesis of MSCs is to promote healing.

Alleviation of pain by non-addictive analgesics while not interfering with healing can have a significant clinical impact as it could reduce the use of NSAIDs and opioids and thus prevent adverse effects of current analgesics on bone healing and potentially combating the opioid crisis. Nevertheless, the unique beneficial features of adenosine to actively promote bone healing while providing pain relief offers a comprehensive and improved approach to address bone trauma and is unmatched by current therapeutics.

What is claimed is:

1. A method of treating, preventing, and/or mitigating pain in a subject, the method comprising:
administering to a subject having pain a biomaterial comprising a therapeutically effective amount of adenosine, wherein the subject's pain is reduced.

2. A method of treating a subject in need of non-opioid analgesia, the method comprising:
administering to a subject having pain a biomaterial comprising a therapeutically effective amount of adenosine; wherein the subject's pain is reduced.

3. The method of claim 1 or claim 2, wherein the therapeutically effective amount of adenosine comprises from about 1 mg/kg to about 30 mg/kg of subject's body weight.

4. The method of claim 1 or claim 2, wherein the biomaterial comprises a macroporous hydrogel.

5. The method of claim 4, wherein the biomaterial comprises from about 0.1 mg to about 1.0 mg of adenosine per 1 mg of macroporous hydrogel.

6. The method of claim 4, wherein the macroporous hydrogel comprises poly(ethylene glycol)-co-6 aminocaproic acid.

7. The method of claim 6, wherein the macroporous hydrogel is functionalized with 3-(acrylamido)phenylboronic acid (3-APBA).

8. The method of claim 1 or claim 2, wherein the subject has an opioid dependence, has recovered from an opioid dependence, or is an active recovery from an opioid dependence.

9. The method of claim 1 or claim 2, wherein the pain comprises acute pain or chronic pain, post-surgical pain, cancer pain, injury pain, or any combination thereof.

10. The method of claim 1 or claim 2, wherein the subject is a non-human mammal or a human.

11. The method of claim 1 or claim 2, wherein the pain is associated with one or more bone fractures.

12. The method of claim 11, wherein the one or more bone fractures are the result of a non-trauma, a disease, or a disorder.

13. The method of claim 11, wherein the one or more bone fractures are the result of an injury, a surgery, or a physical trauma.

14. The method of claim 11, wherein administering comprises implanting the biomaterial at or near the one or more bone fractures.

15. The method of claim 1 or claim 2, wherein the subject's pain is not the result of one or more bone fractures.

16. The method of claim 15, wherein administering comprises implanting the biomaterial at or near the source of the subject's pain.

17. The method of claim 1 or claim 2, further comprising improving the subject's ability to move and/or ambulate.

18. The method of claim 1 or claim 2, further comprising improving the subject's ability to bear weight.

19. The method of claim 1 or claim 2, further comprising reducing functional activity of one or more dorsal root ganglion neurons.

20. The method of claim 19, wherein reducing the functional activity of one or more dorsal root ganglion neurons comprises increasing the level of ADORA1 activation and/or expression.

21. The method of claim 11, further comprising promoting healing of the one or more bone fractures.

22. The method of claim 11, further comprising enhancing the innate ability of bone repair tissue to repair the one or more bone fractures.

23. The method of claim 11, further comprising reducing bone degeneration in the subject and/or promoting bone regeneration in the subject.

24. The method of claim 11, further comprising promoting osteoblastogenesis in or around the area of the one or more bone fractures.

25. The method of claim 24, further comprising increasing the level of ADORA2B activation and/or expression in mesenchymal stromal cells.

26. The method of claim 1 or claim 2, further comprising administering to the subject one or more additional therapeutic agents.

27. The method of claim 26, wherein the additional therapeutic agents comprise one or more non-steroidal anti-inflammatory agents (NSAIDs).

28. The method of claim 26, wherein the additional therapeutic agents comprise one or more opioids.

29. The method of claim 1 or claim 2, further comprising repeating one or more times the administering of the biomaterial.

30. The method of claim 1 or claim 2, wherein the subject's ability to move and/or ambulate is improved, the subject's ability to bear weight is improved, the innate ability of bone repair tissue to repair the one or more bone fractures is enhanced, bone degeneration is decreased, bone regeneration is promoted, osteoblastogenesis in or around the area of the one or more bone fractures is increased, the level of ADORA2B activation and/or ADORA2A expression in mesenchymal stromal cells is increased, the level of ADORA1 activation and/or ADORA1 expression in sensory neurons is increased, or any combination thereof.

31. The method of claim 1 or claim 2, wherein the method does not require administering to the subject one or more opioids.
